(19) United States
(12) Patent Application Publication
(10) Pub. No.: US 2024/0415791 A1
(43) Pub. Date: Dec. 19, 2024

(54) Therapeutic methods and compositions for treating cancer using devimistat and a fatty acid oxidation inhibitor, a tyrosine kinase inhibitor, a glutaminase inhibitor, and/or a glycolysis inhibitor

(71) Applicant: Cornerstone Pharmaceuticals, Inc., Monmouth Junction, NJ (US)

(72) Inventors: Paul Bingham, South Setauket, NY (US); Moises Guardado Rivas, Melville, NY (US); Shawn Stuart, East Moriches, NY (US); Zuzana Zachar, South Setauket, NY (US)

(21) Appl. No.: 18/703,580

(22) PCT Filed: Oct. 21, 2022
(86) PCT No.: PCT/US2022/078539

§ 371 Date: Apr. 22, 2024

Related U.S. Application Data
(60) Provisional application No. 63/262,947, filed on Oct. 22, 2021.

(51) Int. Cl. A61K 31/192; A61K 9/00; A61K 9/08; A61K 9/107; A61K 9/16; A61K 31/4545; A61K 31/4706; A61K 31/5415; A61K 45/06; A61K 47/14; A61K 47/18; A61K 47/26; A61K 47/28; A61K 47/38; A61K 47/44

(52) U.S. Cl. CPC A61K 31/192; A61K 9/0053; A61K 9/08; A61K 9/107; A61K 9/1635; A61K 9/1652; A61K 31/4545; A61K 31/4706; A61K 31/5415; A61K 45/06; A61K 47/14; A61K 47/18; A61K 47/26; A61K 47/28; A61K 47/38; A61K 47/44

(57) ABSTRACT

The invention provides methods and compositions for treating cancer by administering to a patient in need thereof a therapeutically effective amount of devimistat and a fatty acid oxidation inhibitor, a tyrosine kinase inhibitor, a glutaminase inhibitor, and/or a glycolysis inhibitor.

### FIELD OF THE INVENTION

The invention provides methods and compositions for treating cancer.

### BACKGROUND

Devimistat is a first-in-class investigational small-molecule (lipoate analog), which has been evaluated in multiple phase I, I/II, and II clinical studies, and has been granted orphan drug designation for the treatment of pancreatic cancer, acute myeloid leukemia (AML), peripheral T-cell lymphoma (PTCL), Burkitt lymphoma and myelodysplastic syndromes (MDS).

A need exists to improve the safety and efficacy of treating cancer with devimistat. The present invention addresses this need and provides other related advantages.

### SUMMARY

The invention provides methods and compositions for treating cancer in a human patient in need thereof, comprising the step of administering to the patient a therapeutically effective amount of devimistat in combination with a means for inhibiting a cancer cell's utilization of nutrients. In certain embodiments, the invention provides methods and compositions for treating cancer in a human patient in need thereof, comprising the step of administering to the patient a therapeutically effective amount of devimistat in combination with a means for inhibiting fatty acid oxidation in a cancer cell. In certain embodiments, the invention provides methods and compositions for treating cancer in a human patient in need thereof, comprising the step of administering to the patient a therapeutically effective amount of devimistat and a fatty acid oxidation inhibitor, wherein the primary mechanism of action of the fatty acid oxidation inhibitor is not autophagy inhibition. In certain embodiments, the fatty acid oxidation inhibitor is an inhibitor of peroxisomal fatty acid oxidation. In certain embodiments, the fatty acid oxidation inhibitor is an ACOX1 inhibitor. In certain embodiments, the fatty acid oxidation inhibitor is thioridazine. In certain embodiments, the invention provides methods and compositions for treating cancer in a human patient in need thereof, comprising the step of administering to the patient a therapeutically effective amount of devimistat and thioridazine.

The invention further provides methods and compositions for treating cancer in a patient in need thereof, comprising the step of administering to the patient a therapeutically effective amount of devimistat in combination with a means for inhibiting glycolysis in a cancer cell. In certain embodiments, the invention provides methods and compositions for treating cancer in a human patient in need thereof, comprising the step of administering to the patient a therapeutically effective amount of devimistat and a glycolysis inhibitor, wherein the primary mechanism of action of the glycolysis inhibitor is not autophagy inhibition. In certain embodiments, the glycolysis inhibitor is a glycogenolysis inhibitor. In certain embodiments, the glycolysis inhibitor is a glycogen phosphorylase inhibitor. In certain embodiments, the glycolysis inhibitor is 5-chloro-N-[(1S,2R)-3-(dimethylamino)-2-hydroxy-3-oxo-1-(phenylmethyl)propyl]-1H-indole-2-carboxamide. In certain embodiments, the invention provides methods and compositions for treating cancer in a human patient in need thereof, comprising the step of administering to the patient a therapeutically effective amount of devimistat and 5-chloro-N-[(1S,2R)-3-(dimethylamino)-2-hydroxy-3-oxo-1-(phenylmethyl)propyl]-1H-indole-2-carboxamide.

The invention further provides methods and compositions for treating cancer in a patient in need thereof, comprising the step of administering to the patient a therapeutically effective amount of devimistat in combination with a means for inhibiting fatty acid oxidation in a cancer cell and a means for inhibiting glycolysis in a cancer cell. In certain embodiments, the invention provides methods and compositions for treating cancer in a human patient in need thereof, comprising the step of administering to the patient a therapeutically effective amount of devimistat, a fatty acid oxidation inhibitor, and a glycolysis inhibitor, wherein the primary mechanism of action of the fatty acid oxidation inhibitor, the glycolysis inhibitor, or both is not autophagy inhibition. In certain embodiments, the fatty acid oxidation inhibitor is an inhibitor of peroxisomal fatty acid oxidation. In certain embodiments, the fatty acid oxidation inhibitor is an ACOX1 inhibitor. In certain embodiments, the fatty acid oxidation inhibitor is thioridazine. In certain embodiments, the fatty acid oxidation inhibitor is a lipophagy inhibitor. In certain embodiments, the fatty acid oxidation inhibitor is hydroxychloroquine. In certain embodiments, the glycolysis inhibitor is a glycogenolysis inhibitor. In certain embodiments, the glycolysis inhibitor is a glycogen phosphorylase inhibitor. In certain embodiments, the glycolysis inhibitor is 5-chloro-N-[(1S,2R)-3-(dimethylamino)-2-hydroxy-3-oxo-1-(phenylmethyl)propyl]-1H-indole-2-carboxamide. In certain embodiments, the glycolysis inhibitor is an autophagy inhibitor. In certain embodiments, the glycolysis inhibitor is hydroxychloroquine. In certain embodiments, the invention provides methods and compositions for treating cancer in a human patient in need thereof, comprising the step of administering to the patient a therapeutically effective amount of devimistat, thioridazine, and 5-chloro-N-[(1S,2R)-3-(dimethylamino)-2-hydroxy-3-oxo-1-(phenylmethyl)propyl]-1H-indole-2-carboxamide.

The invention further provides methods and compositions for treating cancer in a patient in need thereof, comprising the step of administering to the patient a therapeutically effective amount of devimistat in combination with a means for inhibiting fatty acid oxidation in a cancer cell and a means for inhibiting autophagy in a cancer cell. In certain embodiments, the invention provides methods and compositions for treating cancer in a patient in need thereof, comprising the step of administering to the patient a therapeutically effective amount of devimistat in combination with thioridazine and a means for inhibiting autophagy in a cancer cell. In certain embodiments, the invention provides methods and compositions for treating cancer in a human patient in need thereof, comprising the step of administering to the patient a therapeutically effective amount of devimistat, thioridazine, and an autophagy inhibitor. In certain embodiments, the invention provides methods and compositions for treating cancer in a human patient in need thereof, comprising the step of administering to the patient a therapeutically effective amount of devimistat, thioridazine, and hydroxychloroquine. In certain embodiments, the invention provides methods and compositions for treating cancer in a human patient in need thereof, comprising the step of administering to the patient a therapeutically effective amount of devimistat, thioridazine hydrochloride, and hydroxychloroquine sulfate.

The invention further provides methods and compositions for treating cancer in a patient in need thereof, comprising the step of administering to the patient a therapeutically effective amount of devimistat in combination with a means for inhibiting a tyrosine kinase in a cancer cell. In certain embodiments, the invention further provides methods and compositions for treating cancer in a patient in need thereof, comprising the step of administering to the patient a therapeutically effective amount of devimistat in combination with a means for inhibiting a tyrosine kinase in a cancer cell and a means for inhibiting fatty acid oxidation in a cancer cell. In certain embodiment, the invention provides methods and compositions for treating cancer in a patient in need thereof, comprising the step of administering to the patient a therapeutically effective amount of devimistat in combination with a means for inhibiting a c-met tyrosine kinase in a cancer cell. In certain embodiment, the invention provides methods and compositions for treating cancer in a patient in need thereof, comprising the step of administering to the patient a therapeutically effective amount of devimistat in combination with a means for inhibiting a c-met tyrosine kinase in a cancer cell a means for inhibiting fatty acid oxidation in a cancer cell.

The invention further provides methods and compositions for treating cancer in a patient in need thereof, comprising the step of administering to the patient a therapeutically effective amount of devimistat in combination with a means for inhibiting glutaminase in a cancer cell and at least one means chosen from a means for inhibiting fatty acid oxidation in a cancer cell, a means for inhibiting glycolysis in a cancer cell, a means for inhibiting autophagy in a cancer cell, and a means for inhibiting a tyrosine kinase in a cancer cell. In certain embodiments, the invention provides methods and compositions for treating cancer in a patient in need thereof, comprising the step of administering to the patient a therapeutically effective amount of devimistat in combination with a mean for inhibiting glutaminase in a cancer cell and a means for inhibiting fatty acid oxidation in a cancer cell. In certain embodiments, the invention provides methods and compositions for treating cancer in a patient in need thereof, comprising the step of administering to the patient a therapeutically effective amount of devimistat in combination with a means for inhibiting glutaminase in a cancer cell and a means for inhibiting glycolysis in a cancer cell. In certain embodiments, the invention provides methods and compositions for treating cancer in a patient in need thereof, comprising the step of administering to the patient a therapeutically effective amount of devimistat in combination with a means for inhibiting glutaminase in a cancer cell and a means for inhibiting autophagy in a cancer cell. In certain embodiments, the invention provides methods and compositions for treating cancer in a patient in need thereof, comprising the step of administering to the patient a therapeutically effective amount of devimistat in combination with a means for inhibiting glutaminase in a cancer cell and a means for inhibiting a tyrosine kinase in a cancer cell.

The cancer may be relapsed or refractory. The cancer may be a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. In certain embodiments, the cancer is a lymphoma, leukemia, carcinoma, sarcoma, melanoma, or myeloma. In certain embodiments, the cancer is relapsed or refractory Hodgkin lymphoma, including relapsed or refractory Hodgkin lymphoma in a patient who has failed brentuximab vedotin and a PD-1 inhibitor, relapsed or refractory T-cell non-Hodgkin lymphoma, relapsed or refractory Burkitt's lymphoma, or high-grade B-cell lymphoma with rearrangements of MYC and BCL2 and/or BCL6.

The foregoing aspects of the invention are described in more detail, along with additional embodiments, in the detailed description below.

### BRIEF DESCRIPTION OF THE DRAWINGS

FIG. 1A depicts the effect of media glucose level on cell survival when four cancer cell lines are exposed to various concentrations of devimistat.

FIG. 1B depicts the effect on cell ATP levels of a one-hour pulse of devimistat followed by 19-hour exposure to devimistat in the presence or absence of exogenous glucose in PANC1 and ASPC1 cells.

FIG. 1C depicts the effect of devimistat on glucose uptake and lactate secretion in BxPC3 cells.

FIG. 1D depicts the effect of devimistat on ³H-2-deoxyglucose uptake in four tumor cell lines.

FIG. 2A depicts the effect on cell survival in PANC1 and AsPC1 cells exposed to various concentrations of devimistat for 12 hours, 17 hours, or 21 hours.

FIG. 2B depicts the tumor growth inhibition (TGI) effect of devimistat at various doses in PANC-1 and AsPC-1 xenografts.

FIG. 2C depicts the effect of devimistat on endogenous glycogen stores in PANC1 and AsPC1 cells.

FIG. 2D depicts the level of lipid droplet stores in PANC1 and AsPC1 cells.

FIG. 2E depicts the effect of devimistat on lipid droplet stores in AsPC1 cells.

FIG. 2F depicts the effect of devimistat on acute ATP levels and, after RPMI recovery, commitment to cell death in PANC1, AsPC1, and H460 cells.

FIG. 2G depicts the effect of pre-feeding PANC1 and AsPC1 cells with glucose and/or oleic acid on acute ATP levels and, after RPMI recovery, commitment to cell death, after exposure to devimistat. The bar graphs are alternate plots of the same data for selected devimistat concentrations from the corresponding line graphs.

FIG. 3A depicts the effect of a glycogen phosphorylase inhibitor (CP-91149) and devimistat on ATP levels in PANC1 and AsPC1 cells. The bar graph is an alternate plot of the 100 μM GPi concentration in AsPC1 cells (dashed box in line graph), with statistical significance as indicated.

FIG. 3B depicts the effect of an autophagy inhibitor (hydroxychloroquine; HCQ) and devimistat on ATP levels in PANC1 and AsPC1 cells. The bar graphs are alternate plots of the 100 μM HCQ concentration in PANC1 and AsPC1 cells (dashed boxes in line graphs), with statistical significance as indicated.

FIG. 3C depicts the effect of a fatty acid oxidation inhibitor (thioridazine; TZ) and devimistat on ATP levels in PANC1 and AsPC1 cells. The bar graphs are alternate plots of the 5 μM and 10 μM TZ concentrations in PANC1 and AsPC1 cells, respectively (dashed boxes in line graphs), with statistical significance as indicated.

FIG. 3D in the left and middle bar graphs depicts the effect of devimistat (CPI-613), hydroxychloroquine (HCQ), and CP91149 (GPi) on glycogen consumption in PANC1 and AsPC1 cells. The right bar graph depicts the effect of thioridazine (TZ) and devimistat (CPI-613), alone or in combination, on glycogen consumption in AsPC1 cells.

FIG. 3E depicts the effect of devimistat alone or combined with thioridazine, hydroxychloroquine, and CP91149 on survival of PANC1 and AsPC1 cells in the presence or absence of excess glucose.

FIG. 3F depicts the effects of peroxisomal (thioridazine; TZ) and mitochondrial (etomoxir; ETX) fatty acid beta-oxidation inhibitors, alone or in combination with devimistat, on ATP levels and survival in AsPC1 cells.

FIG. 3G depicts the effects of peroxisomal fatty acid beta-oxidation inhibitor (thioridazine; TZ), alone or in combination with devimistat, on survival in lung cancer (H460), prostate cancer (PC3), and colon cancer (SW260) cells.

FIG. 3H depicts effect of peroxisomal (thioridazine; TZ) and mitochondrial (etomoxir; ETX) fatty acid beta-oxidation inhibitors on oleic acid (OA) or glutamine (GLN) rescue of ATP synthesis in AsPC1 cells treated with devimistat and CP91149.

FIG. 3I depicts effect of peroxisomal (thioridazine; TZ) and mitochondrial (etomoxir; ETX) fatty acid beta-oxidation inhibitors on CO₂ generation in ASPC-1 cells treated with oleic acid.

FIG. 3J depicts the effect of thioridazine (TZ) and devimistat (613; CPI-613), alone or in combination, on AsPC1 xenograft tumor growth.

FIG. 3K depicts the effect of devimistat (CPI-613) and three different doses of thioridazine (TZ), alone or in combination, on AsPC1 xenograft tumor growth.

FIG. 3L depicts the effect of devimistat, oligomycin, BAM, and rotenone on glycogen content in AsPC1 cells.

FIG. 3M depicts a model for the mechanism of devimistat-induced tumor cell starvation/death. ETC indicates the electron transport system. The bold black dashed arrows represent electron flows into the ETC either from the tricarboxylic acid cycle (TCA cycle) or from non-TCA sources (including the fatty acid beta-oxidation step). Details of relevant electron flow sources are as follows: extensive, diverse carbon sources (including pyruvate, amino acid derivatives, and fatty-acid-derived acetate groups) feed the TCA cycle; fatty acid beta-oxidation, itself, also generates reducing equivalents which flow directly to the ETC (including through shuttles from the peroxisome), bypassing the TCA cycle devimistat target; devimistat blocks ATP production dependent on the various TCA cycle-derived electron flows (solid red line); further, these primary devimistat effects drive apparently homeostatic depletion of glucose/glycogen stores, ultimately eliminating their contributions (dashed red line); TZ or CRZ block rescuing electron flows driven by the fatty acid beta-oxidation step, sensitizing otherwise resistant tumors to devimistat under appropriate conditions.

FIG. 3N depicts the effect of anti-sense knockdown of Acox1 protein levels on cell death in AsPC1 cells.

FIG. 4 depicts the anti-tumor efficacy of oral devimistat in human non-small cell lung cancer xenografts in mice.

FIG. 5 depicts the anti-tumor efficacy of oral devimistat in human pancreatic cancer xenografts in mice.

FIG. 6 presents X-ray powder diffraction patterns solid amorphous dispersion formulations of devimistat with either Eudragit L100 or hydroxypropyl methylcellulose acetate succinate (HPMCAS-M) (top and middle diffraction patterns, respectively), and crystalline devimistat (bottom diffraction pattern).

FIG. 7A depicts the effect of phenformin and devimistat, alone and in combination, on ATP levels in AsPC1 cells.

FIG. 7B depicts in the line graph the effects of the cell-permeable form of alpha-ketoglutarate and glutamine on ATP synthesis and induction of cell death in AsPC1 cells in the presence of devimistat and CP91149. The bar graphs depict the effects of the absence (−) or presence (+) of a glutaminase inhibitor (CB-839) or a glutamate dehydrogenase inhibitor (hexachlorophene; HXP) on ATP levels in AsPC1 cells treated with devimistat, CP91149, and either glutamine (GLN) or glucose (GLUC).

FIG. 7C depicts the effect of glutamine (GLN) or glucose (GLC) on survival of PANC-1, PC-3, and H460 cells treated with devimistat.

FIG. 7D depicts the effect on survival of PANC1 and AsPC1 cells treated with a glutaminase inhibitor (CB-839) and/or devimistat.

FIG. 7E depicts the effect of oleic acid, acetate, dimethyl-α-ketoglutarate, or pyruvate on ATP levels in AsPC1 cells treated with devimistat and CP91149.

FIG. 8A depicts the effect of crizotinib (CRZ) on ATP synthesis in AsPC1 cells treated with devimistat and CP91149, and optionally glucose, oleic acid (OA) or glutamine.

FIG. 8B depicts the effect of devimistat (CPI-613) and crizotinib (CRZ), alone or in combination, on survival of two PDAC lines (PANC1, AsPC1) and two lung cancer lines (H460, A549).

FIG. 8C depicts the effect of devimistat (CPI-613) and crizotinib (CRZ), alone or in combination, on AsPC1 xenograft tumor growth.

FIG. 9A depicts the effect of devimistat (613) and a tyrosine kinase inhibitor (PHA6654752, foretinib, PD173955, or GSK1838705A), alone (agent) or in combination (agent+613), on survival of AsPC1 cells and, for PHA6654752 and GSK1838705A, PANC1 cells.

FIG. 9B depicts the effect of a tyrosine kinase inhibitor (PHA6654752, foretinib, PD173955, or GSK1838705A) on glucose, oleic acid, or glutamine rescue of ATP synthesis in AsPC1 cells treated with devimistat and a glycogen phosphorylase inhibitor (CP91149).

### DETAILED DESCRIPTION

The invention provides methods and compositions for treating cancer in a human patient in need thereof, comprising the step of administering to the patient a therapeutically effective amount of devimistat and a fatty acid oxidation inhibitor, wherein the primary mechanism of action of the fatty acid oxidation inhibitor is not autophagy inhibition. The invention further provides methods and compositions for treating cancer in a human patient in need thereof, comprising the step of administering to the patient a therapeutically effective amount of devimistat and a glycolysis inhibitor, wherein the primary mechanism of action of the glycolysis inhibitor is not autophagy inhibition. The invention further provides methods and compositions for treating cancer in a human patient in need thereof, comprising the step of administering to the patient a therapeutically effective amount of devimistat, a fatty acid oxidation inhibitor, and a glycolysis inhibitor, wherein the primary mechanism of action of the fatty acid oxidation inhibitor, the glycolysis inhibitor, or both is not autophagy inhibition.

Various aspects of the invention are set forth below in sections; however, aspects of the invention described in one particular section are not to be limited to any particular section.

### I. Definitions

To facilitate an understanding of the present invention, a number of terms and phrases are defined below.

The term “fatty acid oxidation inhibitor” refers to any agent that slows or inhibits the conversion of fatty acids or lipids into ATP.

The term “glycolysis inhibitor” refers to any agent that slows or inhibits the conversion of glycogen to glucose and/or glucose to ATP.

The term “devimistat” refers to 6,8-bis(benzylsulfanyl)octanoic acid (CPI-613®), having the chemical structure

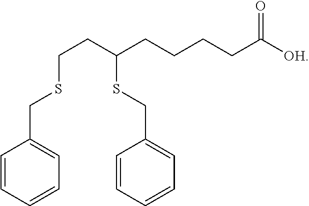 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to a human being in need of cancer treatment.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. In a human or other mammal, a therapeutically effective amount can be determined experimentally in a laboratory or clinical setting, or may be the amount required by the guidelines of the United States Food and Drug Administration, or equivalent foreign agency, for the particular disease and patient being treated. It should be appreciated that determination of proper dosage forms, dosage amounts, and routes of administration is within the level of ordinary skill in the pharmaceutical and medical arts.

As used herein, the term “pharmaceutical composition” refers to the combination of an active agent with an excipient, inert or active, making the composition suitable for administration to a human being.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any pharmaceutical excipient suitable for use in humans. For examples of such excipients, see e.g., Martin, Remington's Pharmaceutical Sciences, 15th Ed., Mack Publ. Co., Easton, PA [1975].

As used herein, the term “pharmaceutically acceptable salt” refers to any salt (e.g., acid or base) of a compound of the present invention which is suitable for administration to a human being. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NR3, wherein R is C1-4 alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, IUPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified.

The phrase “the primary mechanism of action of the [fatty acid oxidation inhibitor or glycolysis inhibitor] is not autophagy inhibition” is employed herein to generally define compounds that are suitable or unsuitable for use in certain embodiments of the present invention. To satisfy this definition and be suitable for use in those embodiments, it should be reasonable to conclude based on available evidence (which may be limited to in vitro, preclinical evidence) that at the therapeutically effective dose the compound does not inhibit fatty acid oxidation or glycolysis by a mechanism that primarily involves inhibition of autophagy. The primary mechanism by which the compound inhibits fatty acid oxidation or glycolysis need not be known, it just needs to be reasonably likely based on available evidence that the mechanism does not primarily involve inhibition of autophagy.

### II. Therapeutic Applications

### Introduction

Devimistat is thought to act as a stable, covalent analog of normally transient catalytic intermediates of the lipoate cofactors of mitochondrial tricarboxylic acid (TCA) cycle-related enzymes, including the pyruvate dehydrogenase (PDH) and α-ketoglutarate dehydrogenase (KGDH) complexes (reviewed in Roche, T. E. and Hiromasa, Y., “Pyruvate dehydrogenase kinase regulatory mechanisms and inhibition in treating diabetes, heart ischemia, and cancer,” Cellular and Molecular Life Sciences, 2007, 64, 830-849; Bingham, P. M. et al., “Lipoic acid and lipoic acid analogs in cancer metabolism and chemotherapy,” Expert Review of Clinical Pharmacology, 2014, 7, 837-846; Solmonson, A. and Deberardinis, R. J., “Lipoic acid metabolism and mitochondrial redox regulation,” Journal of Biological Chemistry, 2018, 293(20), 7522-7530). These lipoate-sensitive regulatory systems, in turn, are apparently significantly reconfigured during evolution of advanced metastatic cancer, creating potential drug targets (reviewed in Bingham et al., 2014).

Devimistat targets tumor regulation of both PDH (carbohydrate carbon TCA entry) and KGDH (glutamine carbon entry and ongoing TCA cycling), producing strong down-regulation of mitochondrial ATP production (Zachar, et al., “Non-redox-active lipoate derivatives disrupt cancer cell mitochondrial metabolism and are potent anticancer agents in vivo,” J. Mol. Med. 2011, 89, 1137-1148; Stuart, S. D. et al., “A strategically designed small molecule attacks alpha-ketoglutarate dehydrogenase in tumor cells through a redox process.” Cancer & Metabolism 2014, 2, 4). The TCA cycle is commonly indispensable for the bulk of tumor cell ATP synthesis; cell viability requires adequate ATP levels. Thus, by inhibiting production of ATP by the TCA cycle devimistat has the potential to impact tumor cell survival.

However, substantial resistance to devimistat single agent treatment is seen in preclinical models (see Examples, below) and patients (Lycan, T W et al., “A phase II clinical trial of CPI-613 in patients with relapsed or refractory small cell lung carcinoma,” Plos One, 2016, 11, e0164244; Alistar A. et al., “Safety and tolerability of the first-in-class agent CPI-613 in combination with modified FOLFIRINOX in patients with metastatic pancreatic cancer: a single-centre, open-label, dose-escalation, phase 1 trial,” Lancet Oncol. 2017, 18, 770-78; Pardee, T. S. et al., “A Phase I Study of the First-in-Class Antimitochondrial Metabolism Agent, CPI-613, in Patients with Advanced Hematologic Malignancies,” Clinical Cancer Research, 2014, 20(20), 5255-5264). In the Examples below the inventors present evidence that such resistance correlates with nutrient availability, including cell-line-specific levels of endogenous nutrient stores. The inventors believe that mobilization of some of these nutrient stores can rescue ATP synthesis and cell survival in the face of devimistat's inhibition of ATP production by the TCA cycle. Specifically, in vitro these endogenous nutrient stores include glycogen, which supports TCA cycle-independent glycolysis, and various other carbon sources, including lipids, capable of feeding electrons directly to the mitochondrial electron transport system (ETC), by-passing the TCA cycle.

The Examples below show that treatment with devimistat drives the rapid, inefficient, presumably homeostatic consumption of these resistance-producing nutrient stores (induced starvation). Although glycolysis may be relatively ineffective in supporting in vivo devimistat resistance in some tumor models, large lipid stores are able to protect a well-characterized resistant PDAC tumor model from devimistat. This lipid-dependent resistance to devimistat may involve tumor-specific reconfiguration of fatty acid catabolism to be significantly dependent on peroxisomal beta-oxidation enroute to mitochondrial oxidation in the presence of devimistat. This peroxisomal role allow practical in vivo targeting. Our in vitro results indicate that this targetable, lipid store-dependent devimistat resistance is general to all tested carcinomas. Finally, the below Examples show that targeting this resistance-producing lipid catabolism in either of two mechanistically independent ways results in xenograft tumor growth inhibition in a PDAC tumor otherwise fully resistant to devimistat.

### Exogenous Nutrient Levels and Experimental Design

Characterizing nutrient levels actually encountered by tumor cells in vivo has proven challenging and occasionally controversial. Approaches using bulk tumor extracellular liquid recovery, potentially including fluids within dysfunctional vascular elements to which tumor cells may have limited access, suggest relatively high levels of free nutrients, including glucose (Sullivan, M R et al., “Quantification of microenvironmental metabolites in murine cancers reveals determinants of tumor nutrient availability,” eLife, 2019, 8, e44235; Siska, P J et al., “Mitochondrial dysregulation and glycolytic insufficiency functionally impair CD8 T cells infiltrating human renal cell carcinoma,” JCI Insight, 2017, 2, e93411; Reinfeld, B I et al., “Cell-programmed nutrient partitioning in the tumor microenvironment,” Nature, 2021, 593, 282-288). In contrast, other approaches attempting to assay tumor fluids in more narrowly focused ways suggest relatively low nutrient levels. Specifically, such measurements indicate extracellular tumor glucose levels of the order ˜100 μM (or substantially lower) compared to ˜1 mM in flanking non-cancerous tissue (Manzo, T et al., “Accumulation of long-chain fatty acids in the tumor microenvironment drives dysfunction in intrapancreatic CD8(+) T cells, J. Exp. Med., 2020, 217, e20191920; Hirayama, A. et al., “Quantitative metabolome profiling of colon and stomach cancer microenvironment by capillary electrophoresis time-of-flight mass spectrometry,” Cancer Research, 2009, 69, 4918-4925, Gullino, P. et al., “The interstitial fluid of solid tumors,” Cancer Research, 1964, 24, 780-797, and Gullino, P. M. et al., “Glucose consumption by transplanted tumors in vivo,” Cancer Research, 1967, 27, 1031-1040).

Our in vivo results are more easily interpreted as indicating that xenograft PDAC tumor cells experience low, limiting access to glucose. Moreover, the strong devimistat induction of exogenous glucose consumption seen in vitro (Examples 1C, 1D, below) suggest that these limiting levels of extracellular tumor glucose in vivo might be rapidly exhausted and thus may play little or no role in drug resistance in vivo. Our in vivo results below corroborate this possibility. Apparently as a result, assaying devimistat activity in the absence of exogenous nutrients proves to be predictive of the drug's in vivo behavior.

Clinical infusion of devimistat produces a transient pulse of serum concentration with a peak in the range of 100 μM (Alistar, et al., 2017). Devimistat levels subsequently fall over several hours after cessation of infusion to the low μM range, slowly declining further over the ensuing ˜24 hours thereafter (op cit.).

In vitro exposure to doses of devimistat spanning this pharmacological range for 8-15 hours in the absence of exogenous nutrients (in carbonate buffered balanced salts) produce variable levels of cell death in tested lines (Example 1A). A brief elevated devimistat pulse mimicking the clinical pattern modestly sensitizes tested cell lines to this killing (Example 1). In contrast, inclusion of 5 mM glucose (approximating glucose levels in serum and some conventional culture media) significantly and similarly protects tested tumor cell lines from this devimistat-induced cell death (Examples 1A and 1). We also note that tumor cell lines display less inter-line response difference and, commonly, approximately one order of magnitude less sensitivity to devimistat in alternative assays employing conventional, nutrient-replete media than under these assay conditions (Zachar et al., 2011).

In view of these results, several of the in vitro studies herein were conducted under low-devimistat-dose and exogenous nutrient-free conditions. Moreover, most studies involve the PANC1 PDAC line (from primary tumor), which is sensitive to devimistat monotherapy, and the AsPC1 PDAC line (from post-treatment metastasis), which is resistant to devimistat monotherapy (Examples 1A, 1B, 2A, and 2B).

Devimistat treatment of all tested carcinoma lines drives accelerated import and apparent glycolytic consumption of exogenous glucose (Examples 1C, D). In the case examined in detail (Example 1C), this accelerated glucose uptake correlates with increased lactate production, suggesting that this (presumably homeostatic) tumor cell response involves increased glycolysis. Glycolysis may therefore sustain ATP synthesis under devimistat inhibition of the TCA cycle.

### Endogenous Nutrient Levels and Devimistat Resistance

When the effects of extracellular nutrients in the media are removed, significant residual quantitative differences in intrinsic devimistat sensitivity between tumor cell lines are revealed (Examples 1A, 1B, 2A and 2F). These in vitro sensitivity differences are similarly observed in vivo, as assessed by xenograft tumor growth inhibition (TGI; Example 2B). The observed differences in sensitivity to devimistat may relate to the endogenous nutrient levels of each cell line.

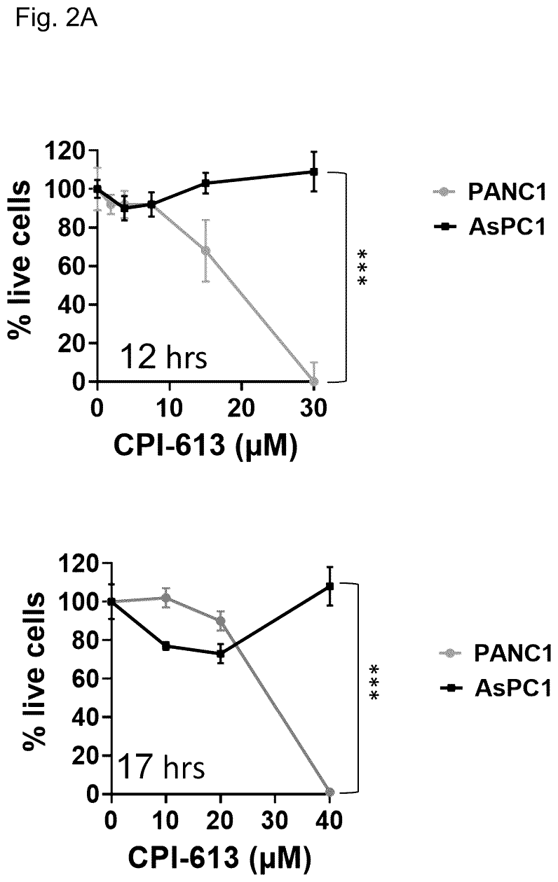

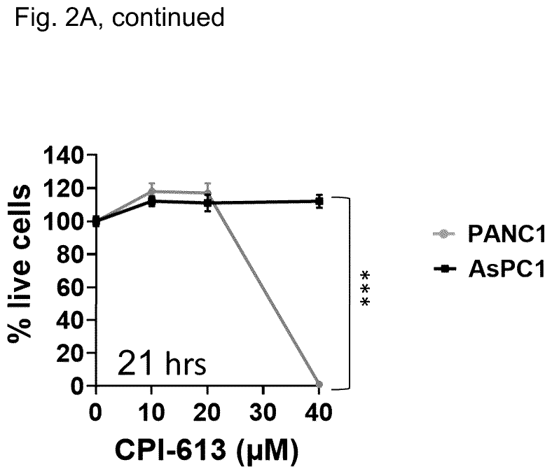

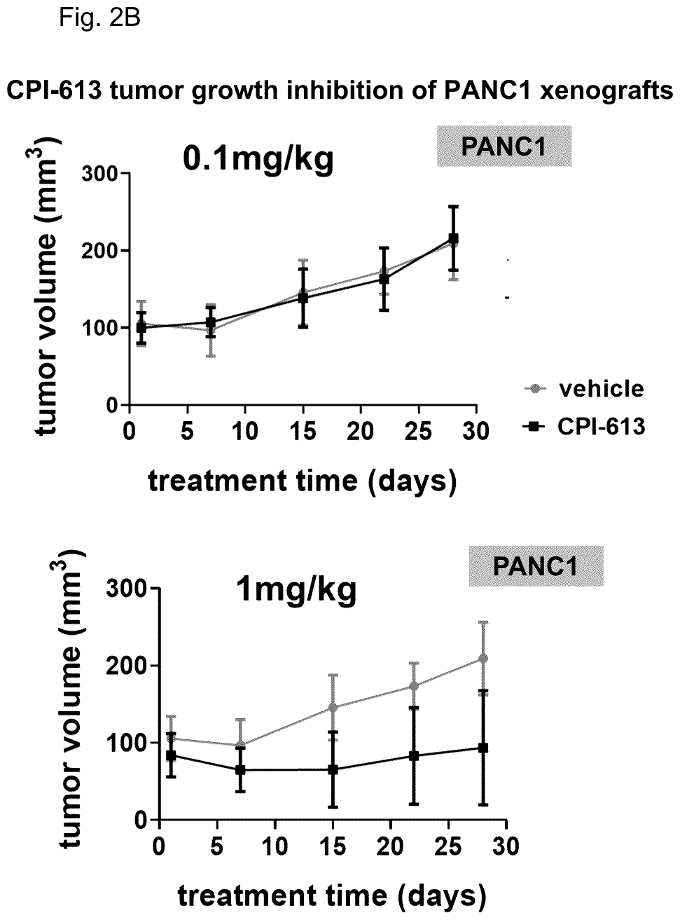

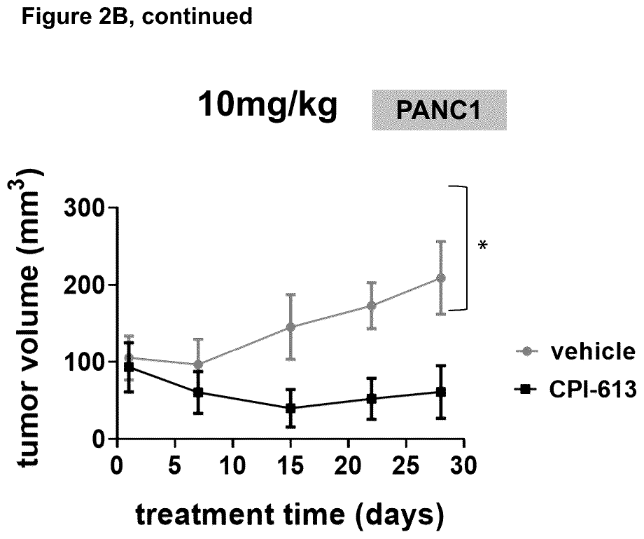

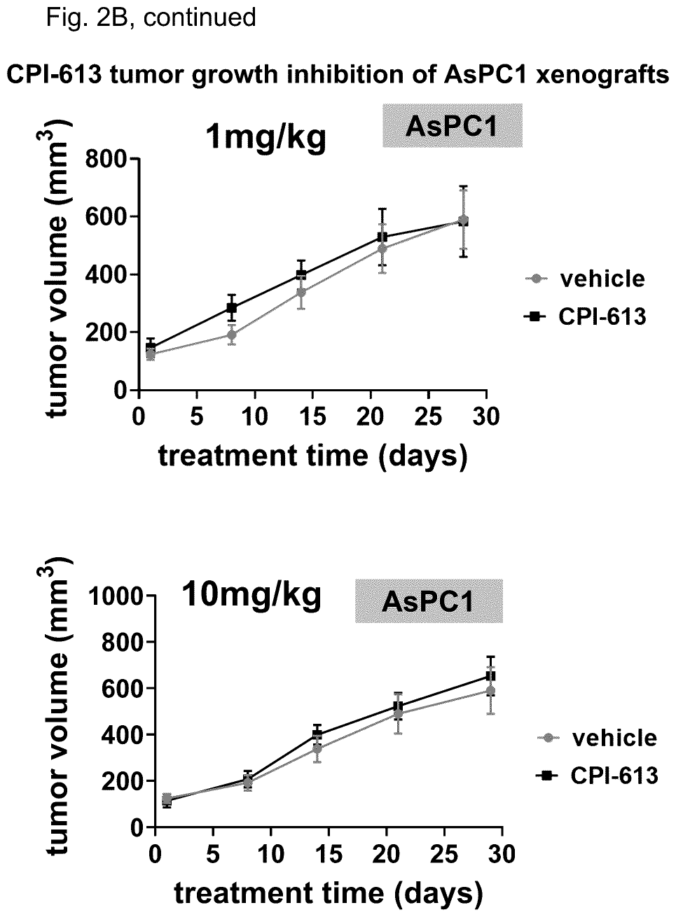

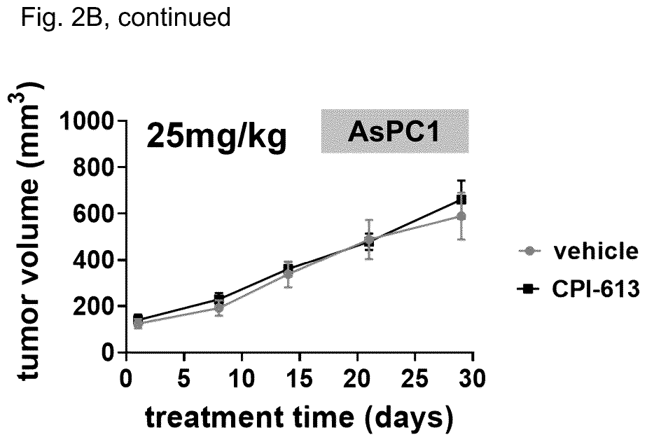

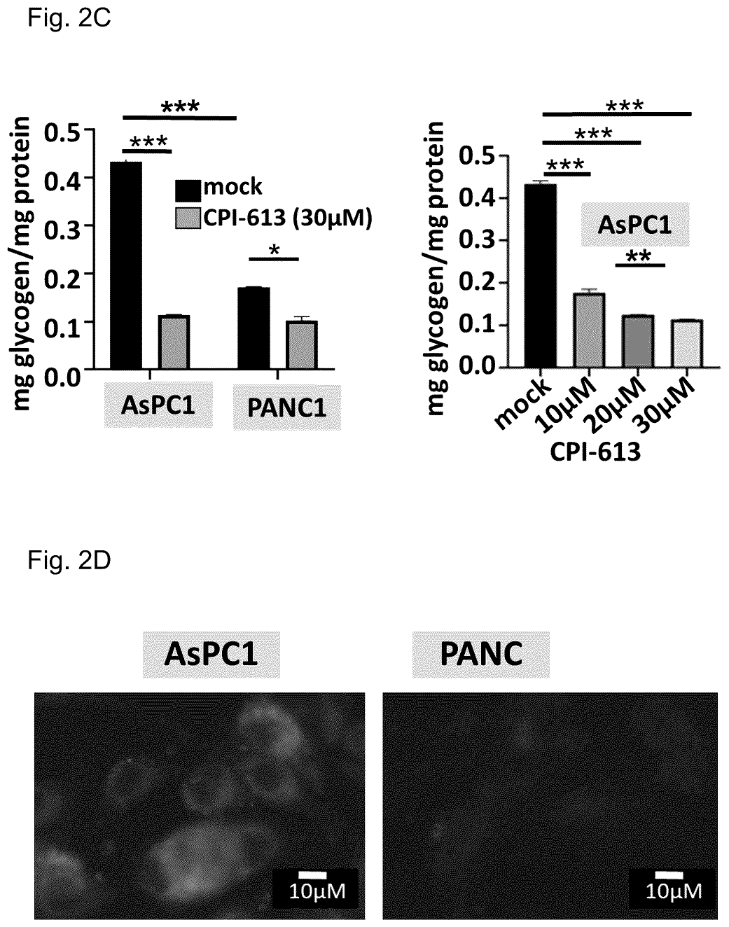

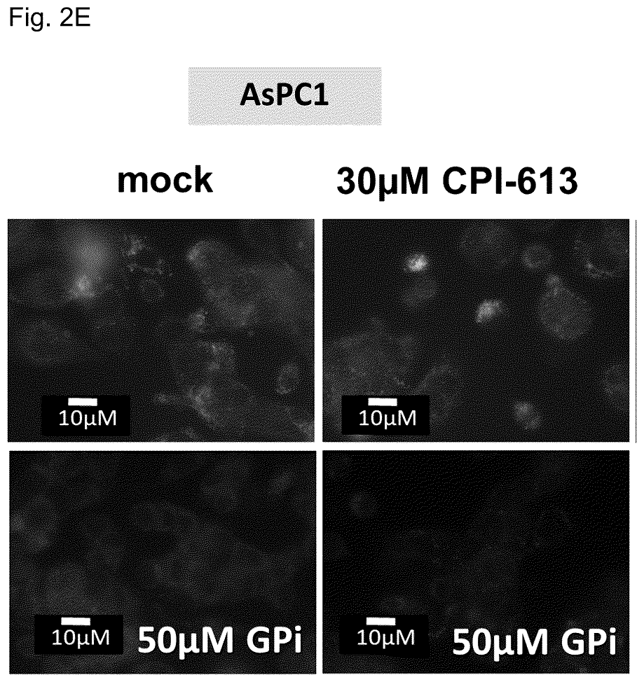

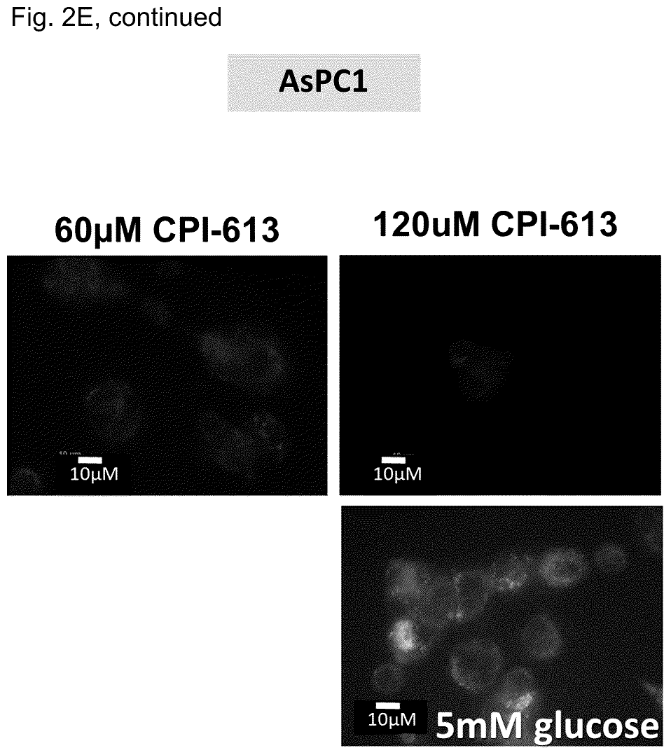

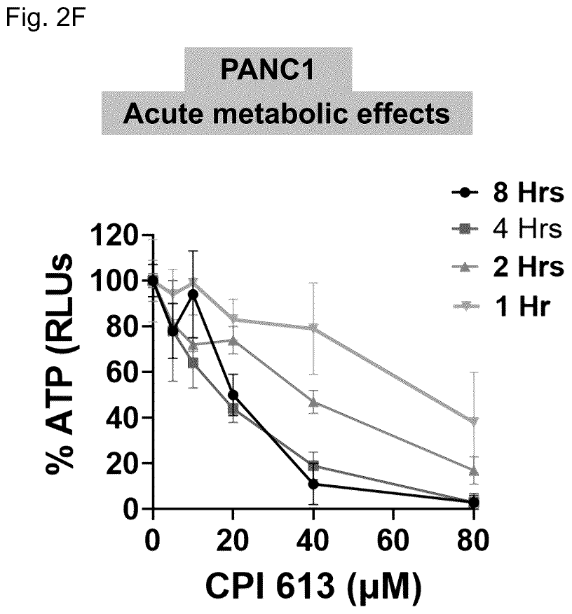

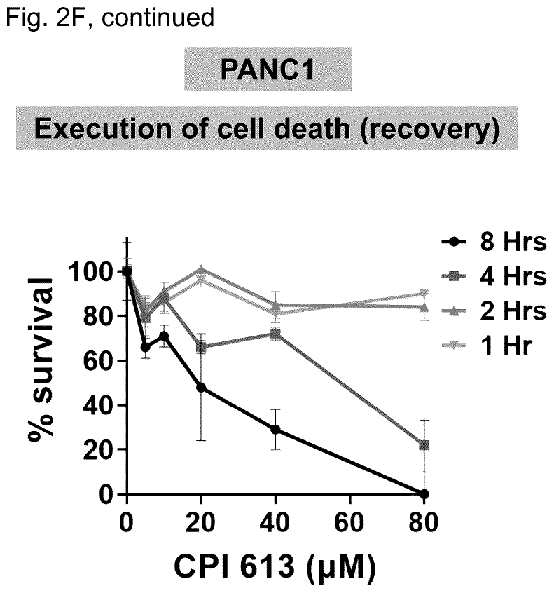

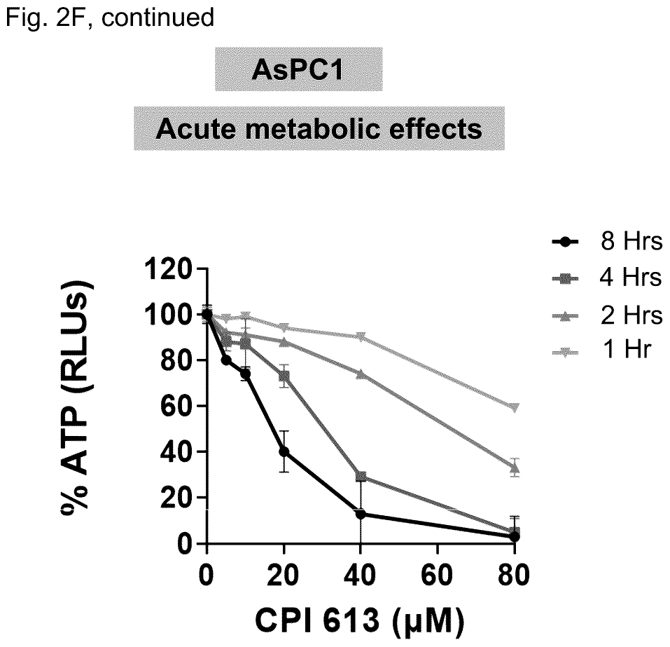

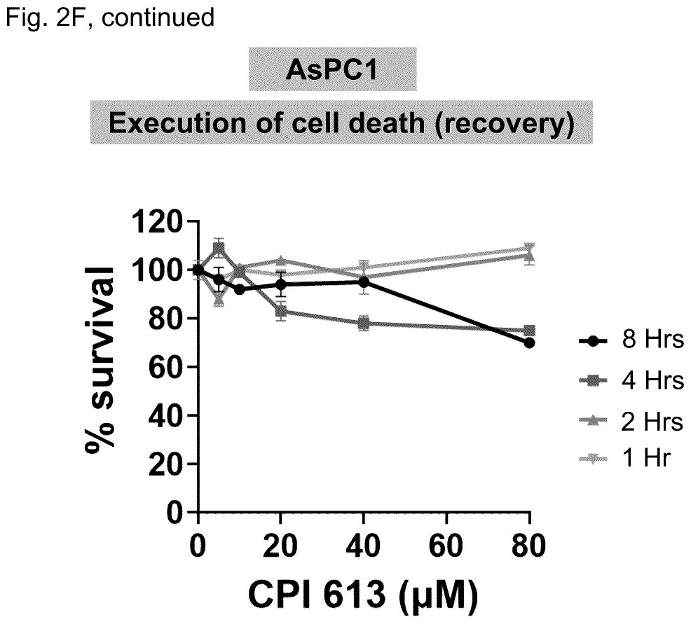

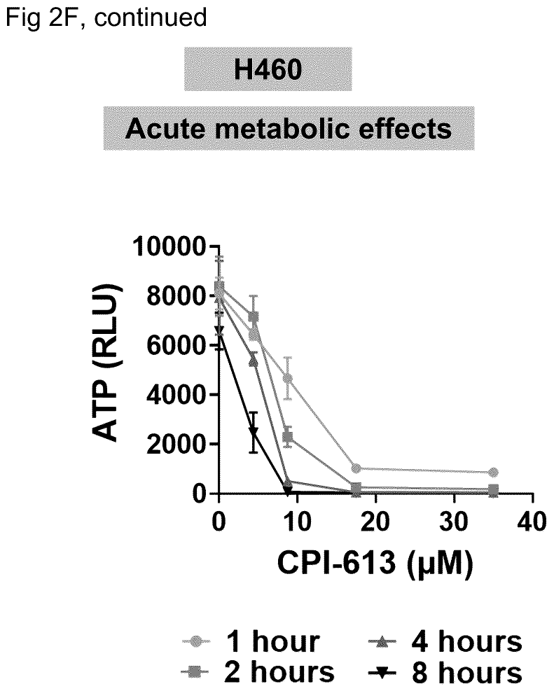

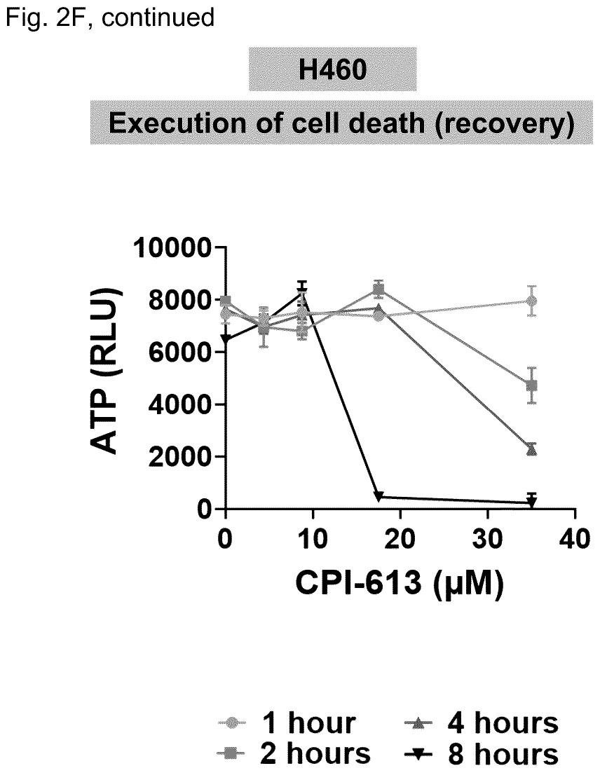

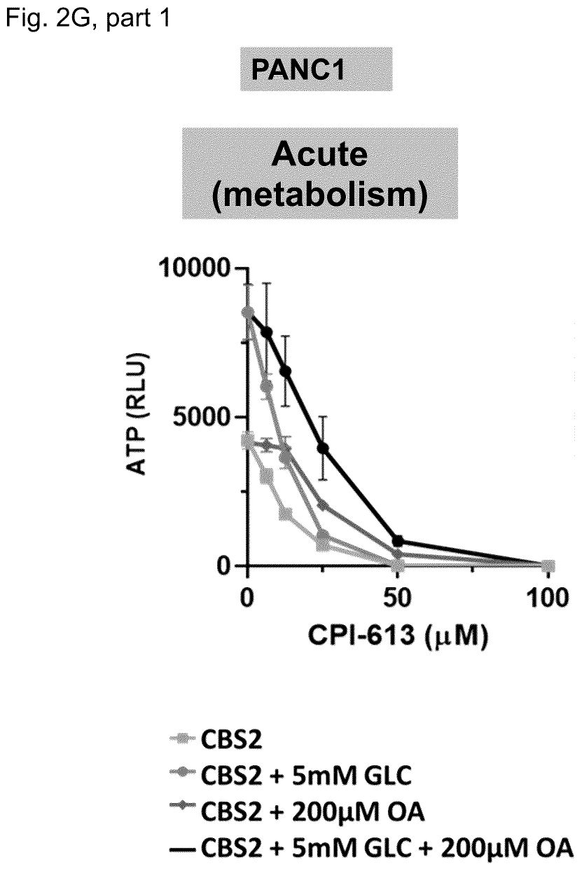

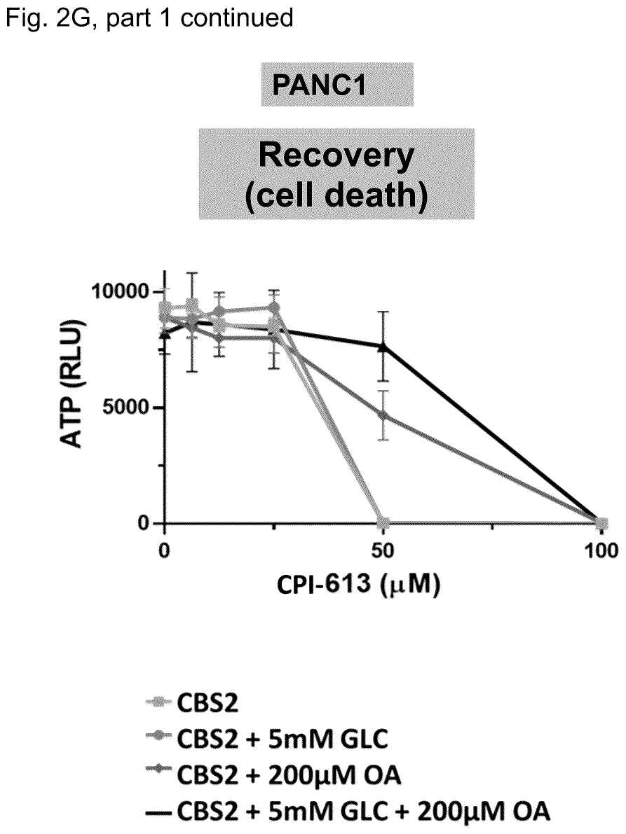

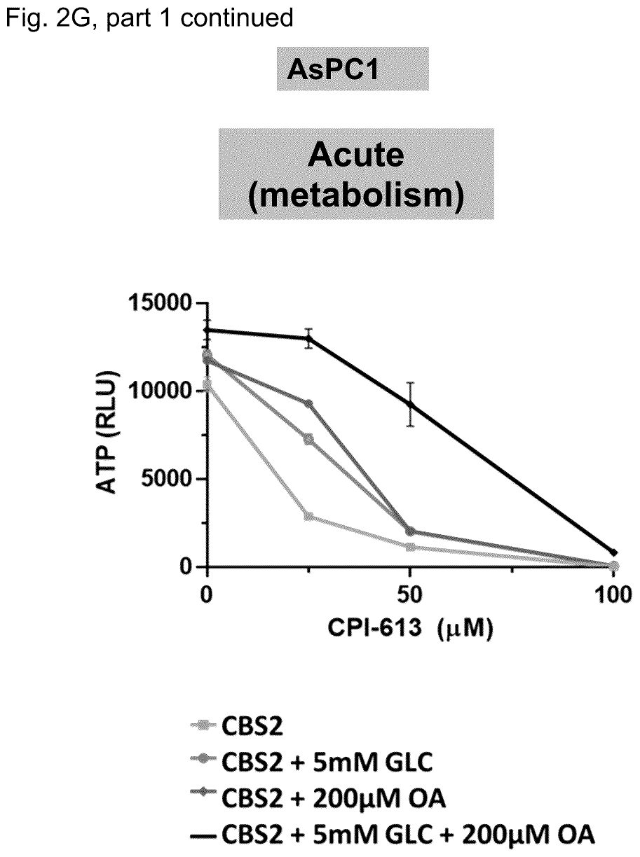

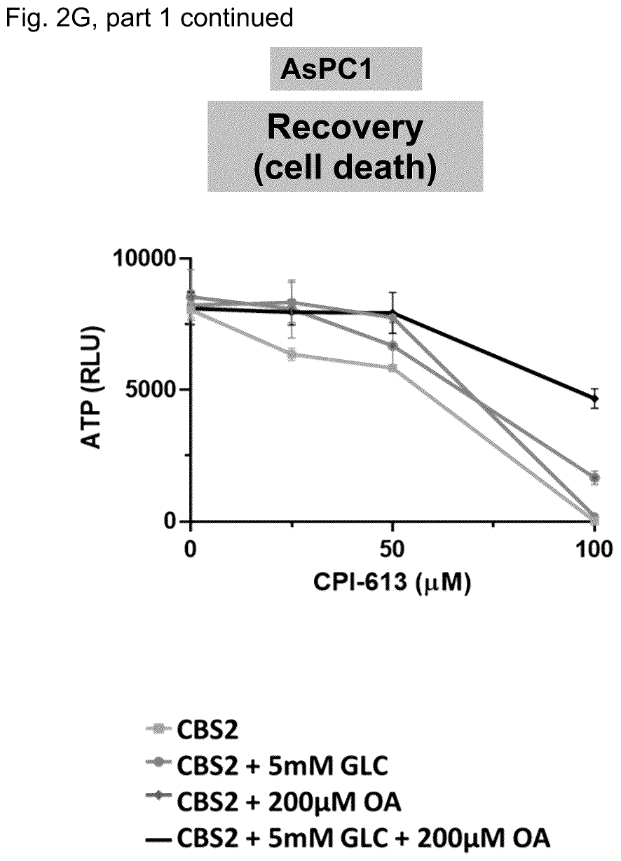

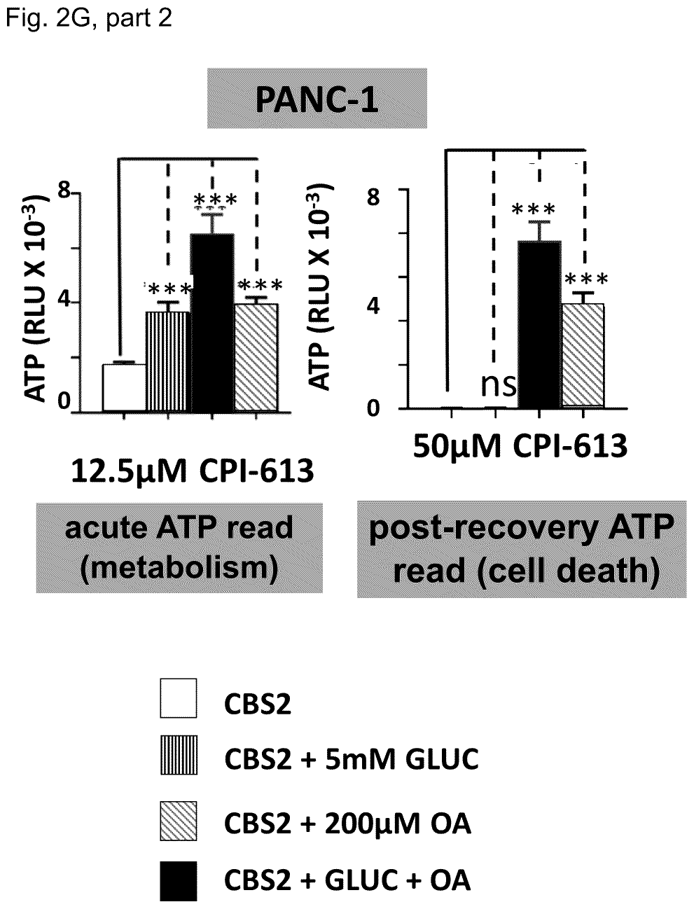

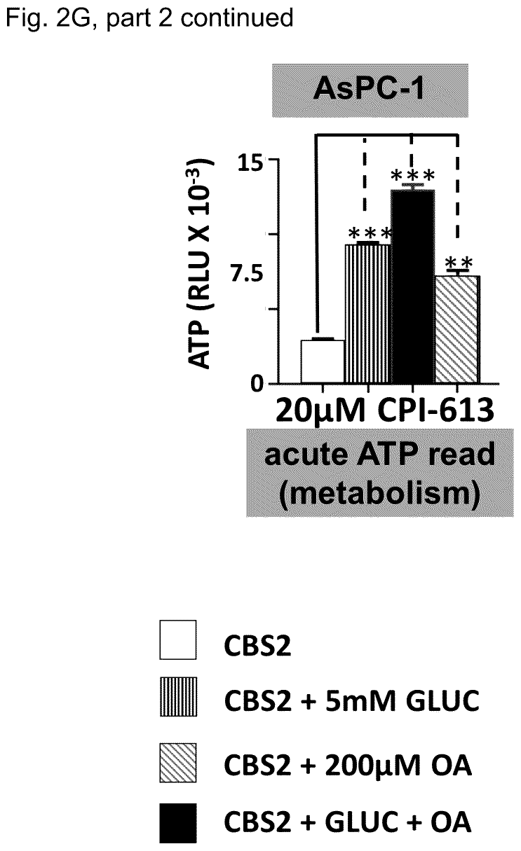

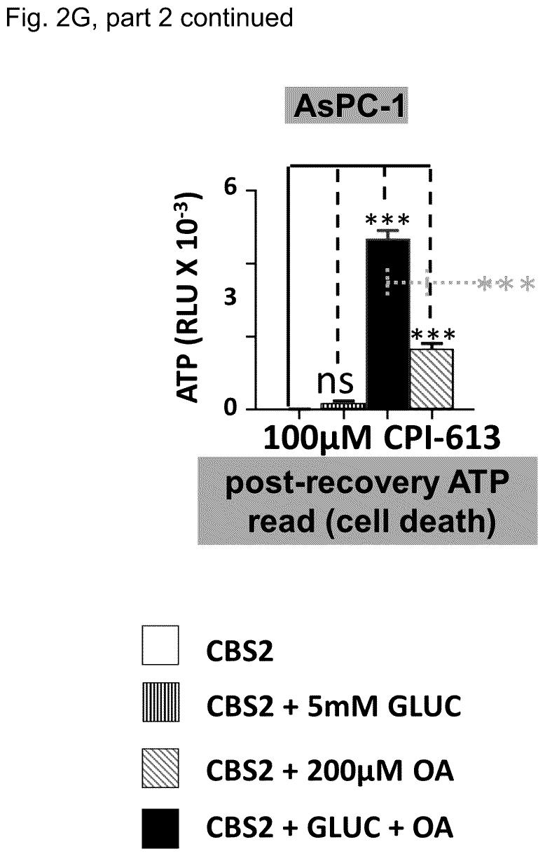

AsPC1 cells, which are resistant to treatment with devimistat, have significantly higher levels of glycogen than the PANC1 line, which are sensitive to devimistat treatment (Example 2C; FIG. 2C, left). Moreover, depletion (mobilization) of glycogen stores is accelerated by exposure to devimistat, presumably providing more glucose to the resistant AsPC1 cells than is available to the sensitive PANC1 cells in vitro (Example 2C; FIG. 2C, left). This devimistat acceleration of glycogen depletion is dose dependent (Example 2C; FIG. 2C, right).

AsPC1 cells also have higher levels of lipid droplet (LD) stores than the sensitive PANC1 line (Example 2D). Moreover, consumption of the elevated AsPC1 LD stores is dose-dependently accelerated by devimistat treatment (Example 2E). This accelerated LD consumption is suppressed in the presence of high levels of exogenous glucose (Example 2E), suggesting that nutrient store catabolism may be coordinated, with LD consumption being reduced prior to exhaustion of carbohydrate stores.

Consistent with this hypothesis, devimistat driven LD consumption is non-linear, perhaps because prior drug-induced glycogen depletion is required (compare 0, 30 μM, and 60 μM doses in FIG. 2D, top row). Moreover, blocking mobilization of AsPC1 glycogen with a phosphorylase inhibitor (GPi; below) results in faster LD depletion, including in response to devimistat (Example 2E). Thus, elevated AsPC1 glycogen levels not only potentially provide higher levels of glucose in response to devimistat than is available in PANC1, these elevated glycogen levels also may delay the onset of consumption of elevated AsPC1 LD stores.

The importance of endogenous nutrient levels to devimistat resistance is supported by experiments revealing that devimistat-induced reduction in ATP synthesis and commitment to cell death are time dependent, and this time dependence differs between store-deficient PANC1 and store-replete AsPC1 (Example 2F). The importance of endogenous nutrient levels is further supported by experiments showing that pre-feeding PDAC cell lines with glucose and/or oleic acid to enhance store formation protects from subsequent treatment with devimistat in the absence of exogenous nutrients (Example 2G). Thus, commitment to tumor cell death induced by devimistat correlates extensively with ATP depletion, which in turn correlates with nutrient store depletion. For simplicity of exposition, the inventors speak herein as if ATP levels are the signal directly used to make these drug-induced cell death decisions. However, the inventors have not ruled out that other signals, for which ATP is a covarying proxy, are also involved. Note further that the comparison of the acute metabolic and cell death effects in these studies indicate that even relatively modest residual ATP levels are sufficient to sustain cell survival (Example 2F).

Collectively, these results represent a diverse body of circumstantial evidence supporting the hypothesis that clinical dose ranges of devimistat may kill tumor cells by inducing accelerated consumption of nutrient resources, followed by starvation, ATP depletion, and commitment to cell death. Moreover, the experiments in Example 2B support the hypothesis that cell line-specific variation in nutrient stores contributes to difference in drug sensitivity in vivo.

### Combination Treatment to Enhance Sensitivity to Devimistat

The inventors further tested the causal role of endogenous nutrient store mobilization in devimistat resistance using small molecule inhibitors of mobilization. First, a well-characterized inhibitor of phosphorolytic mobilization of glycogen stores (CP91149; GPi; Martin, W. H. et al., “Discovery of a human liver glycogen phosphorylase inhibitor that lowers blood glucose in vivo,” Proceedings of the National Academy of Sciences of the United States of America, 1998, 95, 1776-1781) reduces glycogen consumption (Example 3D) and sensitizes tumor cells to devimistat-induced commitment to cell death in the resistant AsPC1 cell line, possessing high levels of glycogen stores (Example 3A). In contrast, this agent interacts more weakly with devimistat in the already sensitive PANC1 cell line, possessing more limited glycogen stores (Example 3A).

Second, etomoxir (ETX) and thioridazine (TZ) are two well-characterized inhibitors of fatty acid catabolism (Divakaruni, A. S. et al., “Etomoxir Inhibits Macrophage Polarization by Disrupting CoA Homeostasis,” Cell Metabolism, 2018, 28(3), 490-; O'Connor, R. S. et al., “The CPT1a inhibitor, etomoxir induces severe oxidative stress at commonly used concentrations,” Scientific Reports, 2018, 8; Raud, B. et al., “Etomoxir Actions on Regulatory and Memory T Cells Are Independent of Cpt1a-Mediated Fatty Acid Oxidation,” Cell Metabolism, 2018, 28(3), 504-; Vandenbranden, C. and Roels, F., “Thioridazine—a selective inhibitor of peroxisomal beta-oxidation in vivo,” Febs Letters, 1985, 187, 331-333; Shi, R. L. et al., “Inhibition of peroxisomal beta-oxidation by thioridazine increases the amount of VLCFAs and A3 generation in the rat brain,” Neuroscience Letters, 2012, 528, 6-10; Yan, H. and Ajuwon, K. M., “Mechanism of butyrate stimulation of triglyceride storage and adipokine expression during adipogenic differentiation of porcine stromovascular cells,” Plos One, 2015, 10, e0145940; Kriska, T. et al., “Deactivation of 12(S)-HETE through (omega-1)-hydroxylation and beta-oxidation in alternatively activated macrophages,” Journal of Lipid Research, 2018, 59, 615-624; Fransen, M. et al., “The peroxisome-mitochondria connection: how and why?” International Journal of Molecular Sciences, 2017, 18(6), 1126; Wanders, R. J. et al., “Metabolic Interplay between Peroxisomes and Other Subcellular Organelles Including Mitochondria and the Endoplasmic Reticulum,” Frontiers in Cell and Developmental Biology, 2016, 3, #83). ETX inhibits the carnitine-dependent mitochondrial import of fatty acids characteristic of abundant serum lipids and endogenous cellular stores, including lipid droplets (op cit.). TZ inhibits the first step in peroxisomal fatty acid beta-oxidation catalyzed by acyl-CoA oxidase (Acox1) specific to this organelle (op cit.).

Both ETX and TZ enhance devimistat sensitivity in AsPC1 cells and TZ also sensitizes all other tested carcinoma lines (Examples 3C, 3F, 3G, 3H, 3J). Moreover, these effects correlate with the ability of both agents to block oleic acid beta-oxidation (Example 3I), and with the ability of TZ to inhibit Acox activity (Example 3N). Each agent also blocks exogenous fatty acid-dependent ATP synthesis in the presence of GPi inhibition of glycogenolysis and devimistat inhibition of the TCA cycle (Example 3H), directly demonstrating that OA can protect from devimistat inhibition of mitochondrial ATP synthesis.

Third, both glycogen and lipid stores can also be mobilized by autophagy (glycophagy or lipophagy, respectively) (Kaur, J. and Debnath, J., “Autophagy at the crossroads of catabolism and anabolism,” Nature Reviews Molecular Cell Biology, 2015, 16, 461-472; Maan, M. et al., “Lipid metabolism and lipophagy in cancer,” Biochemical and Biophysical Research Communications, 2018, 504, 582-589; Condello, M. et al., “Targeting Autophagy to Overcome Human Diseases,” International Journal of Molecular Sciences, 2019, 20(3), 725). Hydroxychloroquine (HCQ) is thought to inhibit late steps in autophagic delivery of such catabolic resources. HCQ interacts strongly with devimistat, enhancing induction of tumor cell commitment to death in both PANC1 and AsPC1 cells in the absence of exogenous nutrients (Example 3B).

Moreover, HCQ treatment significantly accelerates glycogen consumption in both PANC1 and AsPC1 (Example 3D). On the one hand, together with the GPi results above, this result suggests that glycophagy might be relatively unimportant for glycogen mobilization in these two cell lines. On the other hand, some effect of autophagy inhibition, such as lipophagy inhibition, may homeostatically accelerate glycogenolysis.

Fourth, targeting peroxisomal fatty acid beta-oxidation is effective in sensitizing tested carcinoma tumor cell lines to devimistat (Examples 3C, 3F, 3G, 3H). TZ inhibition of fatty acids beta-oxidation also accelerates glycogen consumption, presumably representing a homeostatic response to inhibition of fatty acid catabolism (Example 3D).

Blocking a tumor cell's utilization of glycogen and lipids using a triple combination of the agents discussed above (GPi, TZ, and HCQ) strongly sensitizes the cells to treatment with devimistat, with otherwise resistant AsPC1 cells exhibiting an effect similar to sensitive PANC1 lines (Example 3D).

Collectively, these results suggest that inhibition of mitochondrial metabolism in any way stimulates accelerated nutrient store depletion/consumption. Indeed, three well-characterized, non-cancer-specific mitochondrial metabolism inhibitors (Nelson, D. L. and Cox, M. M., Lehninger Principles of Biochemistry (7^{th} edition), New York Worth, New York, 2017), rotenone (ETC Complex I inhibitor), BAM (Mitchell/Moyle proton gradient uncoupler), and oligomycin (mitochondrial ATP synthase inhibitor) induce accelerated glycogen depletion in AsPC1 cells similar to devimistat (Example 3L).

### Catabolic Electron Flow to the Mitochondrial Electron Transport System (ETC) as a Potential Source of Devimistat Resistance

The rapid, metabolically inefficient consumption of glucose stores in response to devimistat may be explained mechanistically by glycolytic ATP generation (generating about 2 ATPs/mole of glucose), while TCA cycle support of full oxidation of glucose-derived pyruvate (an additional ca. 28 ATPs/mole of glucose) is inhibited by devimistat (Example 1C; Stuart et al., 2014). An analogous mechanism may explain the rapid, inefficient burning of lipids in response to devimistat implied by drug-accelerated lipid store consumption (compare Examples 2E and 2F). Specifically, the initial fatty acid beta-oxidation process, itself, can provide electrons directly to ETC (bypassing the TCA cycle) for ATP synthesis, even if devimistat inhibits efficient TCA cycle-dependent ATP synthesis from oxidation of the resulting acetate units, reducing molar fatty acid ATP yield (Examples 3H, 9E). As discussed above, glycolysis inhibitors (e.g., GPi) and beta oxidation inhibitors (e.g., thioridazine) can block these TCA cycle-independent routes of ATP synthesis and thereby enhance devimistat antitumor activity.

Consistent with this mechanistic hypothesis, inhibition of ETC electron flow by the Complex I inhibitor, phenformin, sensitizes AsPC1 cells with high endogenous lipid stores to devimistat killing (Example 9A). On the other hand, exogenous glutamine, catabolism of which can deliver one electron pair directly to the ETC independently of the TCA cycle (through glutamine dehydrogenase (GDH)) robustly protects from devimistat, analogously to oleic acid rescue. Although glutamine catabolism also delivers α-ketoglutarate directly to the TCA cycle (DeBarardinis R J and Chandel N S, “Fundamentals of cancer metabolism,” Sci Adv., 2016, 2, e1600200), α-ketoglutarate provides no significant protection (Examples 9B, 9E), and glutamine rescue from devimistat inhibition of ATP synthesis and induction of cell death apparently requires GDH electrons that can be delivered directly to the ETC (Example 9B).

Another approach to clinical targeting of lipid-dependent devimistat resistance Tyrosine kinase inhibitors (RTKi's) such as crizotinib (CRZ) are widely deployed in cancer therapy and, in some cases, are thought to influence tumor metabolism (reviewed in Masui, K. et al, “Metabolic reprogramming in the pathogenesis of glioma: Update.” Neuropathology, 2019, 39(1), 3-13). Crizotinib robustly inhibits OA-dependent rescue of ATP synthesis (Example 10A) and sensitizes to devimistat tumor cell killing in vitro and in vivo (Examples 10B, 10D). Like many RTKi's, CRZ is significantly promiscuous. Among its relatively high affinity targets are the ALK, ROS1, and MET kinases (Klaeger, S. et al., “The target landscape of clinical kinase drugs.” Science, 2017, 358(6367)). In vitro assays of selected additional inhibitors with partially overlapping targeting patterns suggests that MET kinase inhibition may contribute significantly to these CRZ lipid catabolic effects (Example 10C).

### Type of Cancer

In certain embodiments, the cancer is associated with altered energy metabolism. As used herein, the term “cancer” is intended to include myelodysplastic syndromes, and in certain embodiments of the present invention the cancer is a myelodysplastic syndrome. In certain embodiments, the cancer is high risk myelodysplastic syndrome (MDS). In certain embodiments, the cancer is high risk MDS in patients who have failed to respond, progressed, or relapsed while on hypomethylating therapy.

The method may be further characterized according to the severity or type of cancer. In certain embodiments, the cancer is Stage I or early stage cancer, in which the cancer is small and only in one area. In certain embodiments, the cancer is Stage II or III, in which the cancer is larger and has grown into nearby tissues or lymph nodes. In certain embodiments, the cancer is Stage IV or advanced or metastatic, in which the cancer has spread to other parts of the body. In certain embodiments, the cancer is resistant to devimistat. In certain embodiments, the cancer is a devimistat-resistant pancreatic cancer.

In certain embodiments, the cancer is Stage I lymphoma, in which the cancer is found in one lymph node region or the cancer has invaded one extra-lymphatic organ or site but not any lymph node regions. In certain embodiments, the cancer is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the cancer is Stage III lymphoma, in which there is cancer in lymph nodes on both sides of the diaphragm. In certain embodiments, the cancer is Stage IV lymphoma, in which the cancer has spread one or more organs beyond the lymph nodes.

In certain embodiments, the cancer is progressive or refractory. In certain embodiments, the cancer is a metastatic. In certain embodiments, the cancer is recurrent or relapsed. In certain embodiments, the cancer is relapsed or refractory. In certain embodiments, the cancer is a T-cell lymphoma. In certain embodiments, the cancer is a B-cell lymphoma. In certain embodiments, the cancer is previously untreated. In certain embodiments, the patient has not received hematopoietic cell transplant. In certain embodiments, the patient has received hematopoietic cell transplant.

In certain embodiments, the cancer is a lymphoma. In certain embodiments, the cancer is mantle cell lymphoma. In certain embodiments, the cancer is a leukemia. In certain embodiments, the cancer is an acute myeloid leukemia. In certain embodiments, the cancer is chronic myeloid leukemia. In certain embodiments, the cancer is acute lymphoblastic leukemia. In certain embodiments, the cancer is a carcinoma. In certain embodiments, the cancer is a sarcoma. In certain embodiments, the cancer is a myeloma. In certain embodiments, the cancer is a clear cell cancer. In certain embodiments, the cancer is a clear cell sarcoma. In certain embodiments, the cancer is a clear cell carcinoma. In certain embodiments, the cancer is a brain or spinal cord cancer. In certain embodiments, the cancer is a melanoma. In certain embodiments, the cancer is a blastoma. In certain embodiments, the cancer is a germ cell tumor. In certain embodiments, the cancer is a cancer of the pancreas. In certain embodiments, the cancer is a metastatic pancreatic cancer. In certain embodiments, the cancer is a locally advanced pancreatic cancer. In certain embodiments, the cancer is a cancer of the prostate. In certain embodiments, the cancer is a castration resistant prostate cancer. In certain embodiments, the cancer is a cancer of the lung. In certain embodiment, the cancer is non-small cell lung cancer. In certain embodiments, the cancer is a cancer of the colon. In certain embodiments, the cancer is a cancer of the rectum. In certain embodiments, the cancer is a colorectal cancer. In certain embodiments, the cancer is a cancer of the cervix. In certain embodiments, the cancer is a neuroendocrine tumor. In certain embodiments, the cancer is a gastroenteropancreatic neuroendocrine tumor. In certain embodiments, the cancer is a cancer of the liver. In certain embodiments, the cancer is a cancer of the uterus. In certain embodiments, the cancer is a cancer of the cervix. In certain embodiments, the cancer is a cancer of the bladder. In certain embodiments, the cancer is a cancer of the kidney. In certain embodiments, the cancer is a cancer of the breast. In certain embodiments, the cancer is a cancer of the ovary. In certain embodiments, the cancer is biliary tract cancer.

In certain embodiments, the cancer is Burkitt's Lymphoma. In certain embodiments, the cancer is relapsed or refractory Burkitt's Lymphoma. In certain embodiments, the cancer is relapsed or refractory Burkitt's Lymphoma in which the patient has failed at least one previous line of therapy. In certain embodiments, the cancer is relapsed or refractory Burkitt's Lymphoma in which the patient has failed prior bone marrow transplant. In certain embodiments, the cancer is double hit diffuse large B cell lymphoma. In certain embodiments, the cancer is high-grade B cell lymphoma with rearrangements of MYC and BCL2 and/or BCL6 (DHL/THL). In certain embodiments, the cancer is Hodgkin lymphoma. In certain embodiments, the cancer is non-Hodgkin lymphoma. In certain embodiments, the cancer is T-cell non-Hodgkin lymphoma. In certain embodiments, the cancer is relapsed or refractory Hodgkin lymphoma. In certain embodiments, the cancer is relapsed or refractory non-Hodgkin lymphoma. In certain embodiments, the cancer is relapsed or refractory T-cell non-Hodgkin lymphoma. In certain embodiments, the cancer is Hodgkin lymphoma in which the patient has not received hematopoietic cell transplant. In certain embodiments, the cancer is Hodgkin lymphoma in which the patient has received hematopoietic cell transplant. In certain embodiments, the cancer is non-Hodgkin lymphoma in which the patient has not received hematopoietic cell transplant. In certain embodiments, the cancer is non-Hodgkin lymphoma in which the patient has received hematopoietic cell transplant. In certain embodiments, the cancer is T-cell non-Hodgkin lymphoma in which the patient has not received hematopoietic cell transplant. In certain embodiments, the cancer is T-cell non-Hodgkin lymphoma in which the patient has received hematopoietic cell transplant. In certain embodiments, the cancer is relapsed or refractory Hodgkin lymphoma in which the patient has not received hematopoietic cell transplant. In certain embodiments, the cancer is relapsed or refractory Hodgkin lymphoma in which the patient has received hematopoietic cell transplant. In certain embodiments, the cancer is relapsed or refractory non-Hodgkin lymphoma in which the patient has not received hematopoietic cell transplant. In certain embodiments, the cancer is relapsed or refractory Hodgkin lymphoma in which the patient has or has not received hematopoietic cell transplant. In certain embodiments, the cancer is relapsed or refractory Hodgkin lymphoma in which the patient has failed brentuximab vedotin and a PD-1 inhibitor. In certain embodiments, the cancer is relapsed or refractory Hodgkin lymphoma in which the patient has failed brentuximab vedotin and a PD-1 inhibitor and has received hematopoietic cell transplant. In certain embodiments, the cancer is relapsed or refractory Hodgkin lymphoma in which the patient has failed brentuximab vedotin and a PD-1 inhibitor and has not received hematopoietic cell transplant. In certain embodiments, the cancer is relapsed or refractory non-Hodgkin lymphoma in which the patient has received hematopoietic cell transplant. In certain embodiments, the cancer is relapsed or refractory T-cell non-Hodgkin lymphoma in which the patient has not received hematopoietic cell transplant. In certain embodiments, the cancer is relapsed or refractory T-cell non-Hodgkin lymphoma in which the patient has received hematopoietic cell transplant. In certain embodiments, the cancer is relapsed or refractory T-cell non-Hodgkin lymphoma in which the patient has or has not received hematopoietic cell transplant.

In certain embodiments, the cancer is a carcinoma, sarcoma, or myeloma. In certain embodiments, the cancer is lymphoma. In certain embodiments, the cancer is leukemia. In certain embodiments, the cancer is a solid tumor.

In certain embodiments, the cancer is bladder cancer, colon cancer, pancreatic cancer, breast cancer, ovarian cancer, prostate cancer, squamous cell carcinoma, basal cell carcinoma, sweat gland carcinoma, sebaceous gland carcinoma, esophagus cancer, lung cancer, stomach cancer, cervical cancer, testicular cancer, skin cancer, rectal cancer, thyroid cancer, kidney cancer, uterus cancer, or liver cancer.

In certain embodiments, the cancer is an acoustic neuroma, oligodendroglioma, meningioma, neuroblastoma, or retinoblastoma. In certain embodiments, the cancer is Burkitt's lymphoma. In certain embodiments, the cancer is a T-cell lymphoma. In certain embodiments, the cancer is acute lymphocytic leukemia or acute myeloid leukemia. In certain embodiments, the cancer is chronic lymphocytic leukemia or chronic myeloid leukemia. In certain embodiments, the cancer is myelodysplastic syndrome or hairy cell leukemia.

In certain embodiments, the cancer is a solid tumor or leukemia. In certain embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain embodiments, the cancer is colon cancer, small-cell lung cancer, non-small cell lung cancer, renal cancer, ovarian cancer, renal cancer, or melanoma.

Additional exemplary cancers include fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is carcinoma, sarcoma, lymphoma, leukemia, germ cell tumor, or blastoma. In certain embodiments, the cancer is a carcinoma or melanoma. In certain embodiments, the cancer is a carcinoma. In certain embodiments, the cancer is a melanoma. In certain embodiments, the cancer is a primary or metastatic melanoma, lung cancer, liver cancer, Hodgkin's lymphoma, non-Hodgkin's lymphoma, uterine cancer, cervical cancer, bladder cancer, kidney cancer, colon cancer, or adenocarcinomas such as breast cancer, prostate cancer, ovarian cancer, or pancreatic cancer. In certain embodiments, the cancer is a breast cancer, a pancreatic cancer, a colon cancer, or a lung cancer. In certain embodiments, the cancer is a pancreatic cancer, a colon cancer, or a lung cancer.

### General Aspects of Administering a Therapeutic Agent to a Patient

Generally, the therapeutic agents—e.g., devimistat, fatty acid oxidation inhibitor, glycolysis inhibitor, tyrosine kinase inhibitor and/or glutaminase inhibitor—are delivered to the patient in a therapeutically effective amount, sufficient for the combination to treat cancer. The treatment may involve one or several administrations on one or more days, and the dosage of each agent may be adjusted by the individual practitioner to achieve a desired effect. Preferably, the dosage amount of each agent should be sufficient to interact primarily with disease cells, leaving normal cells comparatively unharmed.

The dosage amount may be administered in a single dose or in the form of individual divided doses, such as one, two, three, or four times per day. In certain embodiments, the daily dosage amount is administered in a single dose. In the event that the response in a patient is insufficient at a certain dose, higher or more frequent doses may be employed to the extent of patient tolerance.

For the present combination therapy, each agent may be administered in a particular order and/or on the same or different days according to a treatment cycle. For example, a dose of devimistat may be administered to the patient prior to administering a fatty acid oxidation inhibitor and/or glycolysis inhibitor, such as immediately prior, earlier in the day, or on an earlier day in a treatment cycle. In certain embodiments, the active agents may be administered on the same day of a treatment cycle, for example being co-administered simultaneously or one right after the other. In certain embodiments, a dose of a fatty acid oxidation inhibitor and/or glycolysis inhibitor is administered to the patient prior to administering the devimistat, such as immediately prior, earlier in the day, or on an earlier day in a treatment cycle. In certain embodiments, treatment cycles may be repeated one or more times in order to maximize benefit to the patient.

### Devimistat

The devimistat may be administered in any suitable form, including as a solid or liquid, a free acid or salt. The devimistat may be crystalline, amorphous, or dissolved in solution. In certain embodiments, the devimistat is administered to the patient as a salt or ion pair. In certain embodiments, the devimistat is administered to the patient as a salt or ion pair with triethanolamine. Exemplary ion pairing agents that may be used include, for example, a tertiary amine (such as triethylamine or triethanolamine), other amines such as diethylamine, diethanolamine, monoethanolamine, meglumine, mefenamic acid and tromethamine, and combinations thereof. In certain embodiments, the ion pairing agent is an organic Bronsted base. In certain embodiments, the ion pairing agent is an amine compound. In certain embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom. In certain embodiments, the therapeutic agent is a salt of devimistat with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain embodiments, the ion pairing agent is an alkali metal hydroxide or alkaline earth metal hydroxide, such as, for example, cesium hydroxide.

In certain embodiments, the devimistat has a purity of at least about 50% (w/w). In certain embodiments, the devimistat has a purity of at least about 60% (w/w). In certain embodiments, the devimistat has a purity of at least about 70% (w/w). In certain embodiments, the devimistat has a purity of at least about 80% (w/w). In certain embodiments, the devimistat has a purity of at least about 90% (w/w). In certain embodiments, the devimistat has a purity of at least about 95% (w/w). In certain embodiments, the devimistat has a purity of at least about 96% (w/w). In certain embodiments, the devimistat has a purity of at least about 97% (w/w). In certain embodiments, the devimistat has a purity of at least about 98% (w/w). In certain embodiments, the devimistat has a purity of at least about 99% (w/w).

### Fatty Acid Oxidation Inhibitor

In certain embodiments of the present invention, a patient is administered devimistat and a fatty acid oxidation inhibitor, optionally in combination with a glycolysis inhibitor, in an amount and for a time effective to treat cancer, wherein the primary mechanism of action of the fatty acid oxidation inhibitor, the glycolysis inhibitor, or both is not autophagy inhibition. According to these embodiments, the fatty acid oxidation inhibitor and/or the glycolysis inhibitor may be an autophagy inhibitor (i.e., its primary mechanism of action may be autophagy inhibition), but in such case the treatment must further comprise administering a fatty acid oxidation inhibitor and/or glycolysis inhibitor whose primary mechanism of action is not autophagy inhibition.

In certain embodiments, the present invention provides a method for treating cancer in a human patient in need thereof, comprising the step of administering to the patient a therapeutically effective amount of devimistat, a fatty acid oxidation inhibitor, and a tyrosine kinase inhibitor. In certain embodiments, the present invention provides a method for treating cancer in a human patient in need thereof, comprising the step of administering to the patient a therapeutically effective amount of devimistat, a fatty acid oxidation inhibitor, a tyrosine kinase inhibitor, and a glycolysis inhibitor. In certain embodiments, the present invention provides a method for treating cancer in a human patient in need thereof, comprising the step of administering to the patient a therapeutically effective amount of devimistat, a fatty acid oxidation inhibitor, and a glutaminase inhibitor. In certain embodiments, the present invention provides a method for treating cancer in a human patient in need thereof, comprising the step of administering to the patient a therapeutically effective amount of devimistat, a fatty acid oxidation inhibitor, a tyrosine kinase inhibitor, and a glutaminase inhibitor. In certain embodiments, the present invention provides a method for treating cancer in a human patient in need thereof, comprising the step of administering to the patient a therapeutically effective amount of devimistat, a fatty acid oxidation inhibitor, a glycolysis inhibitor, and a glutaminase inhibitor. In certain embodiments, the present invention provides a method for treating cancer in a human patient in need thereof, comprising the step of administering to the patient a therapeutically effective amount of devimistat, a fatty acid oxidation inhibitor, a tyrosine kinase inhibitor, a glycolysis inhibitor, and a glutaminase inhibitor.

The fatty acid oxidation inhibitor is any agent that inhibits the conversion of fatty acids or lipids into ATP. Any suitable fatty acid oxidation inhibitor may be used. In certain embodiments, the fatty acid oxidation inhibitor targets peroxisomal fatty acid beta-oxidation. The fatty acid oxidation inhibitor may inhibit the conversion of fatty acids into ATP by any suitable mechanism. In certain embodiments, the fatty acid oxidation inhibitor is an ACOX1 enzyme inhibitor. In certain embodiments, the fatty acid oxidation inhibitor is thioridazine (TZ; 2-methylmercapto-10-[2-(N-methyl-2-piperidyl) ethyl] phenothiazine). In certain embodiments, the fatty acid oxidation inhibitor is 10,12-tricosadiynoic acid (TDYA). In certain embodiments, the fatty acid oxidation inhibitor is etomoxir.

The fatty acid oxidation inhibitor(s) may be administered in any suitable form, including as a solid or liquid, a free acid or salt. The fatty acid oxidation inhibitor(s) may be crystalline, amorphous, or dissolved in solution. In certain embodiments, the fatty acid oxidation inhibitor(s) is administered to the patient as a salt or ion pair. When the fatty acid oxidation inhibitor is an acidic compound, such as 10,12-tricosadiynoic acid, it may be administered as an ion pair with an inorganic or organic base. When the fatty acid oxidation inhibitor is a basic compound, such as thioridazine, it may be administered as an ion pair with an inorganic or organic acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. In certain embodiments, the therapeutic agent is a salt of a fatty acid oxidation inhibitor with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342. In certain embodiments, the fatty acid oxidation inhibitor is thioridazine hydrochloride.

### Glycolysis Inhibitor

In certain embodiments of the present invention, a patient is administered devimistat and a glycolysis inhibitor, optionally in combination with a fatty acid oxidation inhibitor, in an amount and for a time effective to treat cancer, wherein the primary mechanism of action of the glycolysis inhibitor, the fatty acid oxidation inhibitor, or both is not autophagy inhibition. According to these embodiments, the glycolysis inhibitor and/or the fatty acid oxidation inhibitor may be an autophagy inhibitor (i.e., its primary mechanism of action may be autophagy inhibition), but in such case the treatment must further comprise administering a glycolysis inhibitor and/or fatty acid oxidation inhibitor whose primary mechanism of action is not autophagy inhibition.

In certain embodiments, the present invention provides a method for treating cancer in a human patient in need thereof, comprising the step of administering to the patient a therapeutically effective amount of devimistat, a glycolysis inhibitor, and a tyrosine kinase inhibitor. In certain embodiments, the patient is administered a therapeutically effective amount of devimistat, a glycolysis inhibitor, a tyrosine kinase inhibitor, and a fatty acid oxidation inhibitor. In certain embodiments, the patient is administered a therapeutically effective amount of devimistat, a glycolysis inhibitor, and a glutaminase inhibitor. In certain embodiments, the patient is administered a therapeutically effective amount of devimistat, a glycolysis inhibitor, a glutaminase inhibitor, and a tyrosine kinase inhibitor. In certain embodiments, the patient is administered a therapeutically effective amount of devimistat, a glycolysis inhibitor, a tyrosine kinase inhibitor, a glutaminase inhibitor, and a fatty acid oxidation inhibitor.

The glycolysis inhibitor is any agent that inhibits the production of ATP from glucose or glycogen. Any suitable glycolysis inhibitor may be used. In certain embodiments, the glycolysis inhibitor targets the conversion of glycogen into glucose-1-phosphate. The glycolysis inhibitor may inhibit the conversion of glycogen or glucose into ATP by any suitable mechanism. In certain embodiments, the glycolysis inhibitor is a glycogen phosphorylase enzyme inhibitor. In certain embodiments, the glycolysis inhibitor is chosen from 5-chloro-N-[(1S,2R)-3-(dimethylamino)-2-hydroxy-3-oxo-1-(phenylmethyl)propyl]-1H-indole-2-carboxamide (CP-91149), 2-chloro-4,5-difluoro-N-[[[2-methoxy-5-[[(methylamino)carbonyl]amino]phenyl]amino]carbonyl]-benzamide, (3R,5S)-rel-5-[6-(2,4-dichlorophenyl)hexyl]tetrahydro-3-hydroxy-2-oxo-3-furanacetic acid (SB-204990), 2-deoxy-D-glucose, 2-fluoro-2deoxy-D-glucose, 3-bromopyruvate, 3-Bromopyruvate propyl ester (3-BrOP), 5-thioglucose, and dichloroacetic acid. In certain embodiments, the glycolysis inhibitor is 2-deoxy-D-glucose. In certain embodiments, the glycolysis inhibitor is 2-fluoro-2-deoxy-D-glucose. In certain embodiments, the glycolysis inhibitor is CP-91149 (5-chloro-N-[(1S,2R)-3-(dimethylamino)-2-hydroxy-3-oxo-1-(phenylmethyl)propyl]-1H-indole-2-carboxamide). In certain embodiments, the glycolysis inhibitor is 2-chloro-4,5-difluoro-N-[[[2-methoxy-5-[[(methylamino)carbonyl]amino]phenyl]amino]carbonyl]-benzamide.

The glycolysis inhibitor(s) may be administered in any suitable form, including as a solid or liquid, a free acid or salt. The glycolysis inhibitor(s) may be crystalline, amorphous, or dissolved in solution. In certain embodiments, the glycolysis inhibitor(s) is administered to the patient as a salt or ion pair. When the glycolysis inhibitor is an acidic compound, it may be administered as an ion pair with an inorganic or organic base. When the glycolysis inhibitor is a basic compound, it may be administered as an ion pair with an inorganic or organic acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. In certain embodiments, the therapeutic agent is a salt of a glycolysis inhibitor with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

### Autophagy Inhibitor

In certain embodiments, the fatty acid oxidation inhibitor or the glycolysis inhibitor may be an autophagy inhibitor. In certain embodiments, the present invention provides a method of treating cancer in a human patient in need thereof, comprising the step of administering to the patient a therapeutically effective amount of devimistat, an autophagy inhibitor, and a fatty acid oxidation inhibitor, wherein the primary mechanism of action of the fatty acid oxidation inhibitor is not autophagy inhibition. In certain embodiments, the present invention provides a method of treating cancer in a human patient in need thereof, comprising the step of administering to the patient a therapeutically effective amount of devimistat, an autophagy inhibitor, and a glycolysis inhibitor, wherein the primary mechanism of action of the glycolysis inhibitor is not autophagy inhibition. In certain embodiments, the present invention provides a method of treating cancer in a human patient in need thereof, comprising the step of administering to the patient a therapeutically effective amount of devimistat, an autophagy inhibitor, a fatty acid oxidation inhibitor and a glycolysis inhibitor, wherein the primary mechanisms of action of the fatty acid oxidation inhibitor and the glycolysis inhibitor are not autophagy inhibition. In certain embodiments, the present invention provides a method of treating cancer in a human patient in need thereof, comprising the step of administering to the patient a therapeutically effective amount of devimistat, an autophagy inhibitor, and a tyrosine kinase inhibitor. In certain embodiments, the present invention provides a method of treating cancer in a human patient in need thereof, comprising the step of administering to the patient a therapeutically effective amount of devimistat, an autophagy inhibitor, and a glutaminase inhibitor. In certain embodiments, the present invention provides a method of treating cancer in a human patient in need thereof, comprising the step of administering to the patient a therapeutically effective amount of devimistat, an autophagy inhibitor, a glutaminase inhibitor, and a tyrosine kinase inhibitor. The autophagy inhibitor may inhibit any suitable type of autophagy (e.g., macroautophagy, microautophagy, chaperone-mediated autophagy, mitophagy, glycophagy, or lipophagy), and may do so by any suitable mechanism (e.g., by impacting formation of an autophagosome or its cargo). In certain embodiments, the autophagy inhibitor inhibits glycophagy. In certain embodiments, the autophagy inhibitor inhibits macroautophagy or mitophagy. In certain embodiments, the autophagy inhibitor inhibits macroautophagy. In certain embodiments, the autophagy inhibitor inhibits mitophagy. In certain embodiments, the mitophagy inhibitor is Mdivi-1. In certain embodiments, the mitophagy inhibitor is cyclosporine A. In certain embodiments, the autophagy inhibitor inhibits, microautophagy. In certain embodiments, the autophagy inhibitor inhibits chaperone-mediated autophagy. In certain embodiments, the autophagy inhibitor inhibits lipophagy. Any suitable autophagy inhibitor may be used. In certain embodiments, the autophagy inhibitor is chosen from a 4-aminoquinoline, 3-methyladenine (3-MA, CAS #5142-23-4), MHY1485 (CAS #326914-06-1SP600125), 3-methyl-6-(3-methylpiperidin-1-yl)-3H-purine, 6-Chloro-N-(1-ethylpiperidin-4-yl)-1,2,3,4-tetrahydroacridin-9-amine, 4-(((1-(2-Fluorophenyl)cyclopentyl)-amino)methyl)-2-((4-methylpiperazin-1-yl)methyl)phenol, 6-fluoro-N-[4-fluorobenzyl]quinazolin-4-amine, N-acetyl-L-cysteine, L-asparagine, N2,N4-dibenzylquinazoline-2,4-diamine, (2S,3S)-trans-Epoxysuccinyl-L-leucylamido-3-methylbutane ethyl ester, N-[6-(4-chlorophenoxy)hexyl]-N′-cyano-N″-4-pyridinyl-guanidine, leupeptin, 2-(4-Morpholinyl)-8-phenyl-1(4H)-benzopyran-4-one, 4,6-Di-4-morpholinyl-N-(4-nitrophenyl)-1,3,5-triazin-2-amine, pepstatin A, 2-((5-Bromo-2-((3,4,5-trimethoxyphenyl)amino)pyrimidin-4-yl)oxy)-N-methylbenzamide, 6-Fluoro-N-[(4-fluorophenyl)methyl]-4-quinazolinamine, thapsigargin, amodiaquine, artemisinin, mefloquine, primaquine, piperaquine, quinacrine, U0126, 3-methyladenine, bafilomycin A1, chloroquine, hydroxychloroquine, verteporfin, LY294002, SB202190, SB203580, SC79, and wortmannin. In certain embodiments, the autophagy inhibitor is chosen from chloroquine, hydroxychloroquine, and verteporfin. In certain embodiments, the autophagy inhibitor is chosen from hydroxychloroquine and verteporfin. In certain embodiments, the autophagy inhibitor is a 4-aminoquinoline. In certain embodiments, the autophagy inhibitor is chloroquine. In certain embodiments, the autophagy inhibitor is chloroquine phosphate. In certain embodiments, the autophagy inhibitor is chloroquine sulfate. In certain embodiments, the autophagy inhibitor is chloroquine hydrochloride. In certain embodiments, the autophagy inhibitor is hydroxychloroquine. In certain embodiments, the autophagy inhibitor is hydroxychloroquine sulfate. In certain embodiments, the autophagy inhibitor is verteporfin.

### Tyrosine Kinase Inhibitor

In certain embodiments, the present invention provides a method of treating cancer in a human patient in need thereof, comprising the step of administering to the patient a therapeutically effective amount of devimistat and a tyrosine kinase inhibitor. In certain embodiments, the present invention provides a method of treating cancer in a human patient in need thereof, comprising the step of administering to the patient a therapeutically effective amount of devimistat, a tyrosine kinase inhibitor, and a glycolysis inhibitor. In certain embodiments, the present invention provides a method of treating cancer in a human patient in need thereof, comprising the step of administering to the patient a therapeutically effective amount of devimistat, a tyrosine kinase inhibitor, and a glycolysis inhibitor, wherein the primary mechanism of action of the glycolysis inhibitor is not autophagy inhibition. In certain embodiments, the present invention provides a method of treating cancer in a human patient in need thereof, comprising the step of administering to the patient a therapeutically effective amount of devimistat, a tyrosine kinase inhibitor, and a fatty acid oxidation inhibitor. In certain embodiments, the present invention provides a method of treating cancer in a human patient in need thereof, comprising the step of administering to the patient a therapeutically effective amount of devimistat, a tyrosine kinase inhibitor, and a fatty acid oxidation inhibitor, wherein the primary mechanism of action of the fatty acid oxidation inhibitor is not autophagy inhibition. In certain embodiments, the present invention provides a method of treating cancer in a human patient in need thereof, comprising the step of administering to the patient a therapeutically effective amount of devimistat, a tyrosine kinase inhibitor, a fatty acid oxidation inhibitor and a glycolysis inhibitor, wherein the primary mechanisms of action of the fatty acid oxidation inhibitor and the glycolysis inhibitor are not autophagy inhibition. In certain embodiments, the present invention provides a method of treating cancer in a human patient in need thereof, comprising the step of administering to the patient a therapeutically effective amount of devimistat, a tyrosine kinase inhibitor, and a glutaminase inhibitor.

The tyrosine kinase inhibitor may inhibit any suitable tyrosine kinase (e.g., c-Met, ALK, ROS1) and may do so by any suitable mechanism. In certain embodiments, the tyrosine kinase inhibitor is a c-Met (MET) inhibitor. In certain embodiments, the tyrosine kinase inhibitor is an ALK inhibitor. In certain embodiments, the tyrosine kinase inhibitor is a ROS1 inhibitor.

Any suitable tyrosine kinase inhibitor may be used. In certain embodiments, the tyrosine kinase inhibitor is a c-Met inhibitor chosen from crizotinib ((R)-3-[1-(2,6-Dichloro-3-fluorophenyl)ethoxy]-5-[1-(piperidin-4-yl)-1H-pyrazol-4-yl]pyridin-2-amine), PHA-665752 ((2R)-1-[[5-[(Z)-[5-[[(2,6-Dichlorophenyl)methyl]sulfonyl]-1,2-dihydro-2-oxo-3H-indol-3-ylidene]methyl]-2,4-dimethyl-1H-pyrrol-3-yl]carbonyl]-2-(1-pyrrolidinylmethyl)pyrrolidine), foretinib (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide), tivantinib ((3R,4R)-3-(1-azatricyclo[6.3.1.0^{4,12}]dodeca-2,4,6,8(12)-tetraen-3-yl)-4-(1H-indol-3-yl)pyrrolidine-2,5-dione; (3R,4R)-3-(5,6-dihydro-4H-pyrrolo[3,2,1-ij]quinolin-1-yl)-4-(1H-indol-3-yl)pyrrolidine-2,5-dione), savolitinib (3-[(1S)-1-imidazo[1,2-a]pyridin-6-ylethyl]-5-(1-methylpyrazol-4-yl)triazolo[4,5-b]pyrazine), cabozantinib (1-N-[4-(6,7-dimethoxyquinolin-4-yl)oxyphenyl]-1-N′-(4-fluorophenyl)cyclopropane-1,1-dicarboxamide), tepotinib (3-[1-[[3-[5-[(1-methylpiperidin-4-yl)methoxy]pyrimidin-2-yl]phenyl]methyl]-6-oxopyridazin-3-yl]benzonitrile), capmatinib (2-fluoro-N-methyl-4-[7-(quinolin-6-ylmethyl)imidazo[1,2-b][1,2,4]triazin-2-yl]benzamide), onartuzumab, ficlatuzumab, and rilotumumab. In certain embodiments, the tyrosine kinase inhibitor is a c-Met inhibitor chosen from crizotinib and cabozantinib. In certain embodiments, the tyrosine kinase inhibitor is crizotinib. In certain embodiments, the tyrosine kinase inhibitor is cabozantinib. In certain embodiments, the present invention provides a method of treating cancer in a human patient in need thereof, comprising the step of administering to the patient a therapeutically effective amount of devimistat and crizotinib. In certain embodiments, the present invention provides a method of treating cancer in a human patient in need thereof, comprising the step of administering to the patient a therapeutically effective amount of devimistat, crizotinib, and a fatty acid oxidation inhibitor. In certain embodiments, the present invention provides a method of treating cancer in a human patient in need thereof, comprising the step of administering to the patient a therapeutically effective amount of devimistat, crizotinib, and thioridazine. In certain embodiments, the present invention provides a method of treating cancer in a human patient in need thereof, comprising the step of administering to the patient a therapeutically effective amount of devimistat, crizotinib, and a glycolysis inhibitor. In certain embodiments, the present invention provides a method of treating cancer in a human patient in need thereof, comprising the step of administering to the patient a therapeutically effective amount of devimistat, crizotinib, and an autophagy inhibitor. In certain embodiments, the present invention provides a method of treating cancer in a human patient in need thereof, comprising the step of administering to the patient a therapeutically effective amount of devimistat, crizotinib, and hydroxychloroquine. In certain embodiments, the present invention provides a method of treating cancer in a human patient in need thereof, comprising the step of administering to the patient a therapeutically effective amount of devimistat and cabozantinib. In certain embodiments, the present invention provides a method of treating cancer in a human patient in need thereof, comprising the step of administering to the patient a therapeutically effective amount of devimistat, cabozantinib, and a fatty acid oxidation inhibitor. In certain embodiments, the present invention provides a method of treating cancer in a human patient in need thereof, comprising the step of administering to the patient a therapeutically effective amount of devimistat, cabozantinib, and thioridazine. In certain embodiments, the present invention provides a method of treating cancer in a human patient in need thereof, comprising the step of administering to the patient a therapeutically effective amount of devimistat, cabozantinib, and an autophagy inhibitor. In certain embodiments, the present invention provides a method of treating cancer in a human patient in need thereof, comprising the step of administering to the patient a therapeutically effective amount of devimistat, cabozantinib, and hydroxychloroquine. In certain embodiments, the present invention provides a method of treating cancer in a human patient in need thereof, comprising the step of administering to the patient a therapeutically effective amount of devimistat, cabozantinib, and a glycolysis inhibitor.

### Glutaminase Inhibitor

In certain embodiments, the present invention provides a method of treating cancer in a human patient in need thereof, comprising the step of administering to the patient a therapeutically effective amount of devimistat, a glutaminase inhibitor, and a tyrosine kinase inhibitor. In certain embodiments, the present invention provides a method of treating cancer in a human patient in need thereof, comprising the step of administering to the patient a therapeutically effective amount of devimistat, a glutaminase inhibitor, and a glycolysis inhibitor. In certain embodiments, the present invention provides a method of treating cancer in a human patient in need thereof, comprising the step of administering to the patient a therapeutically effective amount of devimistat, a glutaminase inhibitor, and a fatty acid oxidation inhibitor.

Any suitable glutaminase inhibitor may be used. In certain embodiments, the glutaminase inhibitor is 2-(pyridin-2-yl)-N-(5-(4-(6-(2-(3-(trifluoromethoxy)phenyl)acetamido) pyridazin-3-yl)butyl)-1,3,4-thiadiazol-2-yl)acetamide, also known as telaglenastat or CB-839. In certain embodiments, the glutaminase inhibitor is telaglenastat hydrochloride.

In certain embodiments, the present invention provides a method of treating cancer in a human patient in need thereof, comprising the step of administering to the patient a therapeutically effective amount of devimistat, telaglenastat, and a fatty acid oxidation inhibitor. In certain embodiments, the present invention provides a method of treating cancer in a human patient in need thereof, comprising the step of administering to the patient a therapeutically effective amount of devimistat, telaglenastat, and a glycolysis inhibitor. In certain embodiments, the present invention provides a method of treating cancer in a human patient in need thereof, comprising the step of administering to the patient a therapeutically effective amount of devimistat, telaglenastat, and thioridazine. In certain embodiments, the present invention provides a method of treating cancer in a human patient in need thereof, comprising the step of administering to the patient a therapeutically effective amount of devimistat, telaglenastat, and a tyrosine kinase inhibitor. In certain embodiments, the present invention provides a method of treating cancer in a human patient in need thereof, comprising the step of administering to the patient a therapeutically effective amount of devimistat, telaglenastat, and a crizotinib. In certain embodiments, the present invention provides a method of treating cancer in a human patient in need thereof, comprising the step of administering to the patient a therapeutically effective amount of devimistat, telaglenastat, and cabozantinib.

### Route of Administration

The devimistat, fatty acid oxidation inhibitor, glycolysis inhibitor, and tyrosine kinase inhibitor may each be administered to the patient by any suitable route(s). The route of administration of each therapeutic agent may be the same or different. For example, in certain embodiments, the devimistat, fatty acid oxidation inhibitor, tyrosine kinase inhibitor, glutaminase inhibitor and/or glycolysis inhibitor is administered orally to the patient. In certain embodiments, the devimistat is administered orally to the patient. In certain embodiments, the fatty acid oxidation inhibitor is administered orally to the patient. In certain embodiments, the glycolysis inhibitor is administered orally to the patient. In certain embodiments, the tyrosine kinase inhibitor is administered orally to the patient. In certain embodiments, the glutaminase inhibitor is administered orally to the patient. In certain embodiments, the devimistat, fatty acid oxidation inhibitor, tyrosine kinase inhibitor, glutaminase inhibitor and/or glycolysis inhibitor is administered subcutaneously to the patient. In certain embodiments, the devimistat, fatty acid oxidation inhibitor, tyrosine kinase inhibitor, and/or glycolysis inhibitor is administered intravenously to the patient. In certain embodiments, the devimistat is administered as an IV infusion over two hours. In certain embodiments, the devimistat is administered as an IV infusion over two hours via a central venous catheter.

An advantage of oral dosing of the devimistat is that it permits substantially increased dosing flexibility as compared to IV. In the prior art, devimistat is formulated as a 50 mg/mL solution in 1 M (150 mg/mL) aqueous triethanolamine, which is diluted from 50 mg/mL to as low as 4 mg/mL (e.g., 12.5 mg/mL) with sterile 5% dextrose for injection (D5W) prior to administration as an IV infusion over 30-120 minutes via a central venous catheter. Such an infusion is inconvenient for patients and effectively precludes regimens involving frequent and/or prolonged dosing. Since the half-life of devimistat after IV dosing is only about 1-2 hours (Pardee, T. S. et al., C/in Cancer Res. 2014, 20, 5255-64), more frequent and/or prolonged dosing could advantageously be used to increase the patient's exposure to the drug.

Another advantage of oral dosing is that it makes maintenance therapy feasible. For example, a patient who is treated successfully with first line therapy—with or without devimistat—and whose cancer is in partial or complete remission, may be treated orally with devimistat and a fatty acid oxidation inhibitor (e.g., thioridazine), tyrosine kinase inhibitor (e.g., crizotinib or cabozantinib), and/or a glycolysis inhibitor (e.g., CP-91149, 2-deoxy-D-glucose, or 2-fluoro-2-deoxy-D-glucose) on a chronic basis in order to delay or prevent recurrence. The maintenance treatment may involve, for example, one, two, three, four, or five doses per day of the devimistat and fatty acid oxidation inhibitor and/or glycolysis inhibitor on a regular basis, such as daily or weekly.

### Pharmaceutical Composition

Any suitable pharmaceutical composition may be used to administer the devimistat, fatty acid oxidation inhibitor, tyrosine kinase inhibitor, glutaminase inhibitor, and glycolysis inhibitor to the patient. The therapeutic agents may be administered together in the same pharmaceutical composition (e.g., fixed dose combination) or separately in different pharmaceutical compositions. There is a wide variety of suitable formulations of pharmaceutical compositions of the present invention (see, e.g., Remington: The Science and Practice of Pharmacy, 20^{th} ed., Gennaro et al. Eds., Lippincott Williams and Wilkins, 2000). In certain embodiments, one or more of the therapeutic agents is administered in a pharmaceutical composition that is a dry oral dosage form. In certain embodiments, the pharmaceutical composition is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, colorants, film formers, flavoring agents, preservatives, dosing vehicles, and any combination of any of the foregoing.

The pharmaceutical composition will generally include at least one excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the pharmaceutical composition comprises an excipient in an amount of about 5% to about 99%, such as about 10% to about 85%, by weight of the composition, with the therapeutic agent comprising the remainder. In certain embodiments, pharmaceutically acceptable excipients comprise about 20% to about 80% of the total weight of the composition. In certain embodiments, the pharmaceutical composition comprises the therapeutic agent in an amount of at least about 40% by weight of the composition, with one or more excipients comprising the remainder. In certain embodiments, the pharmaceutical composition comprises the therapeutic agent in an amount of at least about 50% by weight of the composition. In certain embodiments, the pharmaceutical composition comprises the therapeutic agent in an amount of at least about 60% by weight of the composition. In certain embodiments, the pharmaceutical composition comprises the therapeutic agent in an amount of at least about 70% by weight of the composition. In certain embodiments, the pharmaceutical composition comprises the therapeutic agent in an amount of at least about 80% by weight of the composition. In certain embodiments, the pharmaceutical composition comprises the therapeutic agent in an amount of at least about 90% by weight of the composition.

Diluents for solid (e.g., oral) compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid pharmaceutical compositions include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The pharmaceutical composition optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid compositions and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, the pharmaceutical composition comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level. The formulations of the invention may be buffered by the addition of suitable buffering agents.

In certain embodiments of the present invention, the therapeutic agent may be formulated as a pharmaceutically-acceptable oil; liposome; oil-water or lipid-oil-water emulsion or nanoemulsion; or liquid. To facilitate such formulations, the therapeutic agent may be combined with a pharmaceutically-acceptable excipient therefor.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Further examples of pharmaceutical formulations of devimistat are described in U.S. Pat. No. 8,263,653, the entire disclosure of which is incorporated by reference herein.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for parenteral administration comprise one or more therapeutic agent of the invention (devimistat, fatty acid oxidation inhibitor, tyrosine kinase inhibitor, or glycolysis inhibitor) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

In certain embodiments, the pharmaceutical composition of the present invention is a unit dose composition. In certain embodiments, the pharmaceutical composition contains about 1 mg to about 5000 mg of the therapeutic agent. In certain embodiments, the pharmaceutical composition contains about 100 mg to about 3000 mg of the therapeutic agent. In certain embodiments, the pharmaceutical composition contains about 200 mg to about 2000 mg of the therapeutic agent. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of therapeutic agent. In certain embodiments, the pharmaceutical composition contains about 300 mg, 500 mg, 700 mg, or 1000 mg of the therapeutic agent.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

In certain embodiments, the pharmaceutical composition of devimistat comprises triethanolamine and devimistat in a mole ratio of triethanolamine to devimistat of about 10:1 to about 1:10. In certain embodiments, the mole ratio of triethanolamine to devimistat is about 10:1 to about 5:1. In certain embodiments, the mole ratio of triethanolamine to devimistat is about 8:1. In certain embodiments, the pharmaceutical composition comprises a 50 mg/mL solution of devimistat in 1M aqueous triethanolamine. In certain embodiments, the pharmaceutical composition comprises a solution of devimistat in 1M aqueous triethanolamine diluted from 50 mg/mL to as low as 12.5 mg/mL with sterile aqueous 5% dextrose for injection (D5W). In certain embodiments, the pharmaceutical composition comprises a solution of devimistat in 1M aqueous triethanolamine diluted from 50 mg/mL to about 12.5 mg/mL with sterile aqueous 5% dextrose for injection (D5W).

In certain embodiments, the pharmaceutical composition of 2-deoxy-D-glucose or other glycolysis inhibitor is suitable for oral administration, such as a tablet or capsule. In certain embodiments, the pharmaceutical composition of 2-deoxy-D-glucose or other glycolysis inhibitor is suitable for oral administration, such as a tablet or capsule, and comprises about 100 mg to about 5 g of 2-deoxy-D-glucose or other glycolysis inhibitor. In certain embodiments, the pharmaceutical composition of 2-deoxy-D-glucose or other glycolysis inhibitor is suitable for oral administration, such as a tablet or capsule, and comprises about 250 mg to about 2.5 g of 2-deoxy-D-glucose or other glycolysis inhibitor. In certain embodiments, the pharmaceutical composition of 2-deoxy-D-glucose or other glycolysis inhibitor is suitable for oral administration, such as a tablet or capsule, and comprises about 500 mg to about 1 g of 2-deoxy-D-glucose or other glycolysis inhibitor. In certain embodiments, the pharmaceutical composition of 2-deoxy-D-glucose or other glycolysis inhibitor is suitable for oral administration, such as a tablet or capsule, and comprises about 100 mg, 200 mg, 250 mg, 300 mg, 400 mg, 500 mg, 750 mg, or 1 g of 2-deoxy-D-glucose or other glycolysis inhibitor.

Pharmaceutical compositions of thioridazine, crizotinib, and cabozantinib are commercially available. In certain embodiments, the pharmaceutical composition of thioridazine is an oral tablet comprising 10 mg thioridazine hydrochloride. In certain embodiments, the pharmaceutical composition of thioridazine is an oral tablet comprising 25 mg thioridazine hydrochloride. In certain embodiments, the pharmaceutical composition of thioridazine is an oral tablet comprising 50 mg thioridazine hydrochloride. In certain embodiments, the pharmaceutical composition of thioridazine is an oral tablet comprising 100 mg thioridazine hydrochloride. In certain embodiments, the pharmaceutical composition of crizotinib is an oral capsule comprising 250 mg crizotinib. In certain embodiments, the pharmaceutical composition of crizotinib is an oral capsule comprising 200 mg crizotinib. In certain embodiments, the pharmaceutical composition of cabozantinib is an oral tablet comprising 25 mg cabozantinib maleate (equivalent to 20 mg cabozantinib). In certain embodiments, the pharmaceutical composition of cabozantinib is an oral tablet comprising 51 mg cabozantinib maleate (equivalent to 40 mg cabozantinib). In certain embodiments, the pharmaceutical composition of cabozantinib is an oral tablet comprising 76 mg cabozantinib maleate (equivalent to 60 mg cabozantinib).

In certain embodiments, the pharmaceutical composition of the glutaminase inhibitor is an oral tablet comprising telaglenastat, such as in an amount of from about 150 mg to about 250 mg, about 250 mg to about 350 mg, about 350 mg to about 450 mg, about 450 mg to about 550 mg, about 550 mg to about 650 mg, about 650 mg to about 750 mg, or about 750 mg to about 850 mg.

### Dosing Amounts & Schedules

The devimistat, fatty acid oxidation inhibitor, tyrosine kinase inhibitor, glutaminase inhibitor, and glycolysis inhibitor may be administered to the patient in any suitable dose according to any suitable schedule. The dose and schedule will vary based on the cancer being treated and can be readily determined by those of ordinary skill in the art in view of the doses and schedules used in the prior art when the therapeutic agents of the invention were administered alone or in combination with other agents, as well as the guidance provided herein. In certain embodiments, the dose is the maximum tolerated dose.

In certain embodiments, the devimistat is administered at a daily dose of about 25 mg/m² to about 5000 mg/m². In certain embodiments, the devimistat is administered at a daily dose of about 50 mg/m² to about 4000 mg/m². In certain embodiments, the devimistat is administered at a daily dose of about 100 mg/m² to about 3000 mg/m². In certain embodiments, the devimistat is administered at a daily dose of about 150 mg/m² to about 3000 mg/m². In certain embodiments, the devimistat is administered at a daily dose of about 250 mg/m² to about 2500 mg/m². In certain embodiments, the first devimistat is administered at a daily dose of about 500 mg/m² to about 2000 mg/m². In certain embodiments, the devimistat is administered at a daily dose of about 25 mg/m². In certain embodiments, the devimistat is administered at a daily dose of about 500 mg/m². In certain embodiments, the devimistat is administered at a daily dose of about 100 mg/m². In certain embodiments, the devimistat is administered at a daily dose of about 150 mg/m². In certain embodiments, the devimistat is administered at a daily dose of about 200 mg/m². In certain embodiments, the devimistat is administered at a daily dose of about 250 mg/m². In certain embodiments, the devimistat is administered at a daily dose of about 300 mg/m². In certain embodiments, the devimistat is administered at a daily dose of about 350 mg/m². In certain embodiments, the devimistat is administered at a daily dose of about 400 mg/m². In certain embodiments, the devimistat is administered at a daily dose of about 450 mg/m². In certain embodiments, the devimistat is administered at a daily dose of about 500 mg/m². In certain embodiments, the devimistat is administered at a daily dose of about 600 mg/m². In certain embodiments, the devimistat is administered at a daily dose of about 700 mg/m². In certain embodiments, the devimistat is administered at a daily dose of about 800 mg/m². In certain embodiments, the devimistat is administered at a daily dose of about 900 mg/m². In certain embodiments, the devimistat is administered at a daily dose of about 1000 mg/m². In certain embodiments, the devimistat is administered at a daily dose of about 1100 mg/m². In certain embodiments, the devimistat is administered at a daily dose of about 1200 mg/m². In certain embodiments, the devimistat is administered at a daily dose of about 1300 mg/m². In certain embodiments, the devimistat is administered at a daily dose of about 1400 mg/m². In certain embodiments, the devimistat is administered at a daily dose of about 1500 mg/m². In certain embodiments, the devimistat is administered at a daily dose of about 1600 mg/m². In certain embodiments, the devimistat is administered at a daily dose of about 1700 mg/m². In certain embodiments, the devimistat is administered at a daily dose of about 1800 mg/m². In certain embodiments, the devimistat is administered at a daily dose of about 1900 mg/m². In certain embodiments, the devimistat is administered at a daily dose of about 2000 mg/m². In certain embodiments, the devimistat is administered at a daily dose of about 2500 mg/m². In certain embodiments, the devimistat is administered at a daily dose of about 3000 mg/m².

In certain embodiments, the devimistat is administered at a daily dose of about 1 mg to about 10,000 mg. In certain embodiments, the devimistat is administered at a daily dose of about 10 mg to about 7,500 mg. In certain embodiments, the devimistat is administered at a daily dose of about 100 mg to about 5,000 mg. In certain embodiments, the devimistat is administered at a daily dose of about 200 mg to about 4,000 mg. In certain embodiments, the devimistat is administered at a daily dose of about 300 mg to about 3,000 mg. In certain embodiments, the devimistat is administered at a daily dose of about 400 mg to about 2,500 mg. In certain embodiments, the devimistat is administered at a daily dose of about 500 mg to about 2,000 mg. In certain embodiments, the devimistat is administered at a daily dose of about 100 mg. In certain embodiments, the devimistat is administered at a daily dose of about 200 mg. In certain embodiments, the devimistat is administered at a daily dose of about 300 mg. In certain embodiments, the devimistat is administered at a daily dose of about 400 mg. In certain embodiments, the devimistat is administered at a daily dose of about 500 mg. In certain embodiments, the devimistat is administered at a daily dose of about 600 mg. In certain embodiments, the devimistat is administered at a daily dose of about 700 mg. In certain embodiments, the devimistat is administered at a daily dose of about 800 mg. In certain embodiments, the devimistat is administered at a daily dose of about 900 mg. In certain embodiments, the devimistat is administered at a daily dose of about 1,000 mg. In certain embodiments, the devimistat is administered at a daily dose of about 1,250 mg. In certain embodiments, the devimistat is administered at a daily dose of about 1,500 mg. In certain embodiments, the devimistat is administered at a daily dose of about 1,750 mg. In certain embodiments, the devimistat is administered at a daily dose of about 2,000 mg. In certain embodiments, the devimistat is administered at a daily dose of about 2,500 mg. In certain embodiments, the devimistat is administered at a daily dose of about 3,000 mg. In certain embodiments, the devimistat is administered at a daily dose of about 3,500 mg. In certain embodiments, the devimistat is administered at a daily dose of about 4,000 mg. In certain embodiments, the devimistat is administered at a daily dose of about 4,500 mg. In certain embodiments, the devimistat is administered at a daily dose of about 5,000 mg. In certain embodiments, the devimistat is administered at a daily dose of about 6,000 mg. In certain embodiments, the devimistat is administered at a daily dose of about 7,000 mg. In certain embodiments, the devimistat is administered at a daily dose of about 8,000 mg. In certain embodiments, the devimistat is administered at a daily dose of about 9,000 mg. In certain embodiments, the devimistat is administered at a daily dose of about 10,000 mg.

The daily dose of devimistat may be administered as one dose or divided into two or more doses—e.g., b.i.d. (two times a day), t.i.d. (three times a day), q.i.d. (four times a day). In certain embodiments, the daily dose may be split into five doses administered in regular intervals during one day. In certain embodiments, the daily dose may be split into six doses administered in regular intervals during one day. At higher daily doses and/or when administered orally or subcutaneously, it will often be beneficial to administer the daily dose of devimistat in multiple doses, e.g., b.i.d., t.i.d., or q.i.d. Since devimistat has a relatively short half-life in the blood, splitting the daily dose may improve efficacy by prolonging exposure time and may also improve safety by reducing peak plasma concentration.

The devimistat may be administered pursuant to a treatment schedule that includes days in which a dose of devimistat is administered and days in which a dose of devimistat is not administered. For example, the devimistat may be administered pursuant to a schedule in which devimistat is administered during the early days of a cycle and then not administered during the latter portion of the cycle. In certain embodiments, the devimistat is administered on days 1-5 of a 28-day cycle. In certain embodiments, the devimistat is administered on days 1, 8, and 15 of a four-week cycle. In certain embodiments, the devimistat is administered on days 1 and 3 of a two-week cycle. In certain embodiments, the devimistat is administered on days 1-5 of a three-week cycle. In certain embodiments, the devimistat is administered on days 1-5 of a two-week cycle. In certain embodiments, the devimistat is administered on days 1-3 of a three-week cycle. In certain embodiments, the devimistat is administered on days 1-3 of a two-week cycle. In certain embodiments, the devimistat is administered every day. In certain embodiments, the devimistat is administered every other day. In certain embodiments, the devimistat is administered three days per week. In certain embodiments, the devimistat is administered two days per week. In certain embodiments, the devimistat is administered one day per week.

In certain embodiments, thioridazine hydrochloride or other fatty acid oxidation inhibitor is administered at a daily dose of about 10 mg to about 1500 mg. In certain embodiments, thioridazine hydrochloride or other fatty acid oxidation inhibitor is administered at a daily dose of about 50 mg to about 1000 mg. In certain embodiments, thioridazine hydrochloride or other fatty acid oxidation inhibitor is administered at a daily dose of about 100 mg to about 1000 mg. In certain embodiments, thioridazine hydrochloride or other fatty acid oxidation inhibitor is administered at a daily dose of about 100 mg to about 800 mg. In certain embodiments, thioridazine hydrochloride or other fatty acid oxidation inhibitor is administered at a daily dose of about 150 mg to about 800 mg. In certain embodiments, thioridazine hydrochloride or other fatty acid oxidation inhibitor is administered at a daily dose of about 150 mg to about 500 mg. In certain embodiments, thioridazine hydrochloride or other fatty acid oxidation inhibitor is administered at a daily dose of about 150 mg to about 300 mg. In certain embodiments, thioridazine hydrochloride or other fatty acid oxidation inhibitor is administered at a daily dose of about 50 mg. In certain embodiments, thioridazine hydrochloride or other fatty acid oxidation inhibitor is administered at a daily dose of about 100 mg. In certain embodiments, thioridazine hydrochloride or other fatty acid oxidation inhibitor is administered at a daily dose of about 150 mg. In certain embodiments, thioridazine hydrochloride or other fatty acid oxidation inhibitor is administered at a daily dose of about 200 mg. In certain embodiments, thioridazine hydrochloride or other fatty acid oxidation inhibitor is administered at a daily dose of about 300 mg. In certain embodiments, thioridazine hydrochloride or other fatty acid oxidation inhibitor is administered at a daily dose of about 400 mg. In certain embodiments, thioridazine hydrochloride or other fatty acid oxidation inhibitor is administered at a daily dose of about 500 mg. In certain embodiments, thioridazine hydrochloride or other fatty acid oxidation inhibitor is administered at a daily dose of about 600 mg. In certain embodiments, thioridazine hydrochloride or other fatty acid oxidation inhibitor is administered at a daily dose of about 700 mg. In certain embodiments, thioridazine hydrochloride or other fatty acid oxidation inhibitor is administered at a daily dose of about 800 mg. In certain embodiments, thioridazine hydrochloride or other fatty acid oxidation inhibitor is administered at a daily dose of about 900 mg. In certain embodiments, thioridazine hydrochloride or other fatty acid oxidation inhibitor is administered at a daily dose of about 1,000 mg. In certain embodiments, thioridazine hydrochloride or other fatty acid oxidation inhibitor is administered at a daily dose of about 1,200 mg. In certain embodiments, thioridazine hydrochloride or other fatty acid oxidation inhibitor is administered at a daily dose of about 1,300 mg. In certain embodiments, thioridazine hydrochloride or other fatty acid oxidation inhibitor is administered at a daily dose of about 1,400 mg. In certain embodiments, thioridazine hydrochloride or other fatty acid oxidation inhibitor is administered at a daily dose of about 1,500 mg.

In certain embodiments, thioridazine hydrochloride or other fatty acid oxidation inhibitor is administered at a daily dose of about 0.25 mg/kg to about 25 mg/kg. In certain embodiments, thioridazine hydrochloride or other fatty acid oxidation inhibitor is administered at a daily dose of about 0.5 mg/kg to about 20 mg/kg. In certain embodiments, thioridazine hydrochloride or other fatty acid oxidation inhibitor is administered at a daily dose of about 0.5 mg/kg to about 15 mg/kg. In certain embodiments, thioridazine hydrochloride or other fatty acid oxidation inhibitor is administered at a daily dose of about 0.5 mg/kg to about 10 mg/kg. In certain embodiments, thioridazine hydrochloride or other fatty acid oxidation inhibitor is administered at a daily dose of about 0.5 mg/kg to about 5 mg/kg. In certain embodiments, thioridazine hydrochloride or other fatty acid oxidation inhibitor is administered at a daily dose of about 0.5 mg/kg to about 3 mg/kg. In certain embodiments, thioridazine hydrochloride or other fatty acid oxidation inhibitor is administered at a daily dose of about 0.5 mg/kg. In certain embodiments, thioridazine hydrochloride or other fatty acid oxidation inhibitor is administered at a daily dose of about 1 mg/kg. In certain embodiments, thioridazine hydrochloride or other fatty acid oxidation inhibitor is administered at a daily dose of about 2 mg/kg. In certain embodiments, thioridazine hydrochloride or other fatty acid oxidation inhibitor is administered at a daily dose of about 3 mg/kg. In certain embodiments, thioridazine hydrochloride or other fatty acid oxidation inhibitor is administered at a daily dose of about 4 mg/kg. In certain embodiments, thioridazine hydrochloride or other fatty acid oxidation inhibitor is administered at a daily dose of about 5 mg/kg. In certain embodiments, thioridazine hydrochloride or other fatty acid oxidation inhibitor is administered at a daily dose of about 6 mg/kg. In certain embodiments, thioridazine hydrochloride or other fatty acid oxidation inhibitor is administered at a daily dose of about 7 mg/kg. In certain embodiments, thioridazine hydrochloride or other fatty acid oxidation inhibitor is administered at a daily dose of about 8 mg/kg. In certain embodiments, thioridazine hydrochloride or other fatty acid oxidation inhibitor is administered at a daily dose of about 9 mg/kg. In certain embodiments, thioridazine hydrochloride or other fatty acid oxidation inhibitor is administered at a daily dose of about 10 mg/kg. In certain embodiments, thioridazine hydrochloride or other fatty acid oxidation inhibitor is administered at a daily dose of about 11 mg/kg. In certain embodiments, thioridazine hydrochloride or other fatty acid oxidation inhibitor is administered at a daily dose of about 12 mg/kg. In certain embodiments, thioridazine hydrochloride or other fatty acid oxidation inhibitor is administered at a daily dose of about 13 mg/kg. In certain embodiments, thioridazine hydrochloride or other fatty acid oxidation inhibitor is administered at a daily dose of about 14 mg/kg. In certain embodiments, thioridazine hydrochloride or other fatty acid oxidation inhibitor is administered at a daily dose of about 15 mg/kg. In certain embodiments, thioridazine hydrochloride or other fatty acid oxidation inhibitor is administered at a daily dose of about 16 mg/kg. In certain embodiments, thioridazine hydrochloride or other fatty acid oxidation inhibitor is administered at a daily dose of about 17 mg/kg. In certain embodiments, thioridazine hydrochloride or other fatty acid oxidation inhibitor is administered at a daily dose of about 18 mg/kg. In certain embodiments, thioridazine hydrochloride or other fatty acid oxidation inhibitor is administered at a daily dose of about 19 mg/kg. In certain embodiments, thioridazine hydrochloride or other fatty acid oxidation inhibitor is administered at a daily dose of about 20 mg/kg. In certain embodiments, thioridazine hydrochloride or other fatty acid oxidation inhibitor is administered at a daily dose of about 21 mg/kg. In certain embodiments, thioridazine hydrochloride or other fatty acid oxidation inhibitor is administered at a daily dose of about 22 mg/kg. In certain embodiments, thioridazine hydrochloride or other fatty acid oxidation inhibitor is administered at a daily dose of about 23 mg/kg. In certain embodiments, thioridazine hydrochloride or other fatty acid oxidation inhibitor is administered at a daily dose of about 24 mg/kg. In certain embodiments, thioridazine hydrochloride or other fatty acid oxidation inhibitor is administered at a daily dose of about 25 mg/kg.

The daily dose of thioridazine hydrochloride or other fatty acid oxidation inhibitor may be administered as one dose or divided into two or more doses—e.g., t.i.d. In certain embodiments, the daily dose of thioridazine hydrochloride or other fatty acid oxidation inhibitor is administered as one dose. In certain embodiments, the daily dose of thioridazine hydrochloride or other fatty acid oxidation inhibitor is divided into two doses and administered b.i.d. In certain embodiments, the daily dose of thioridazine hydrochloride or other fatty acid oxidation inhibitor is divided into three doses and administered t.i.d. In certain embodiments, the daily dose of thioridazine hydrochloride or other fatty acid oxidation inhibitor is divided into four doses and administered q.i.d.

In certain embodiments, the 2-deoxy-D-glucose or other glycolysis inhibitor is administered at a daily dose of about 1 mg/kg to about 100 mg/kg. In certain embodiments, the 2-deoxy-D-glucose or other glycolysis inhibitor is administered at a daily dose of about 5 mg/kg to about 60 mg/kg. In certain embodiments, the 2-deoxy-D-glucose or other glycolysis inhibitor is administered at a daily dose of about 10 mg/kg to about 50 mg/kg. In certain embodiments, the 2-deoxy-D-glucose or other glycolysis inhibitor is administered at a daily dose of about 10 mg/kg. In certain embodiments, the 2-deoxy-D-glucose or other glycolysis inhibitor is administered at a daily dose of about 15 mg/kg. In certain embodiments, the 2-deoxy-D-glucose or other glycolysis inhibitor is administered at a daily dose of about 20 mg/kg. In certain embodiments, the 2-deoxy-D-glucose or other glycolysis inhibitor is administered at a daily dose of about 25 mg/kg. In certain embodiments, the 2-deoxy-D-glucose or other glycolysis inhibitor is administered at a daily dose of about 30 mg/kg. In certain embodiments, the 2-deoxy-D-glucose or other glycolysis inhibitor is administered at a daily dose of about 35 mg/kg. In certain embodiments, the 2-deoxy-D-glucose or other glycolysis inhibitor is administered at a daily dose of about 40 mg/kg. In certain embodiments, the 2-deoxy-D-glucose or other glycolysis inhibitor is administered at a daily dose of about 45 mg/kg. In certain embodiments, the 2-deoxy-D-glucose or other glycolysis inhibitor is administered at a daily dose of about 50 mg/kg. In certain embodiments, the 2-deoxy-D-glucose or other glycolysis inhibitor is administered at a daily dose of about 55 mg/kg. In certain embodiments, the 2-deoxy-D-glucose or other glycolysis inhibitor is administered at a daily dose of about 60 mg/kg. In certain embodiments, the 2-deoxy-D-glucose or other glycolysis inhibitor is administered at a daily dose of about 65 mg/kg. In certain embodiments, the 2-deoxy-D-glucose or other glycolysis inhibitor is administered at a daily dose of about 70 mg/kg. In certain embodiments, the 2-deoxy-D-glucose or other glycolysis inhibitor is administered at a daily dose of about 75 mg/kg.

The daily dose of 2-deoxy-D-glucose or other glycolysis inhibitor may be administered as one dose or divided into two or more doses—e.g., t.i.d. In certain embodiments, the daily dose of 2-deoxy-D-glucose or other glycolysis inhibitor is administered as one dose. In certain embodiments, the daily dose of 2-deoxy-D-glucose or other glycolysis inhibitor is divided into two doses and administered b.i.d. In certain embodiments, the daily dose of 2-deoxy-D-glucose or other glycolysis inhibitor is divided into three doses and administered t.i.d. In certain embodiments, the daily dose of 2-deoxy-D-glucose or other glycolysis inhibitor is divided into four doses and administered q.i.d.

In certain embodiments, hydroxychloroquine sulfate is administered at a daily dose of about 50 mg to about 1500 mg. In certain embodiments, hydroxychloroquine sulfate is administered at a daily dose of about 100 mg to about 1500 mg. In certain embodiments, hydroxychloroquine sulfate is administered at a daily dose of about 200 mg to about 1200 mg. In certain embodiments, hydroxychloroquine sulfate is administered at a daily dose of about 300 mg to about 1200 mg. In certain embodiments, hydroxychloroquine sulfate is administered at a daily dose of about 400 mg to about 1200 mg. In certain embodiments, hydroxychloroquine sulfate is administered at a daily dose of about 600 mg to about 1200 mg. In certain embodiments, hydroxychloroquine sulfate is administered at a daily dose of about 600 mg to about 1000 mg. In certain embodiments, hydroxychloroquine sulfate is administered at a daily dose of about 100 mg. In certain embodiments, hydroxychloroquine sulfate is administered at a daily dose of about 200 mg. In certain embodiments, hydroxychloroquine sulfate is administered at a daily dose of about 300 mg. In certain embodiments, hydroxychloroquine sulfate is administered at a daily dose of about 400 mg. In certain embodiments, hydroxychloroquine sulfate is administered at a daily dose of about 500 mg. In certain embodiments, hydroxychloroquine sulfate is administered at a daily dose of about 600 mg. In certain embodiments, hydroxychloroquine sulfate is administered at a daily dose of about 700 mg. In certain embodiments, hydroxychloroquine sulfate is administered at a daily dose of about 800 mg. In certain embodiments, hydroxychloroquine sulfate is administered at a daily dose of about 900 mg. In certain embodiments, hydroxychloroquine sulfate is administered at a daily dose of about 1,000 mg. In certain embodiments, hydroxychloroquine sulfate is administered at a daily dose of about 1,100 mg. In certain embodiments, hydroxychloroquine sulfate is administered at a daily dose of about 1,200 mg. In certain embodiments, hydroxychloroquine sulfate is administered at a daily dose of about 1,300 mg. In certain embodiments, hydroxychloroquine sulfate is administered at a daily dose of about 1400 mg. In certain embodiments, hydroxychloroquine sulfate is administered at a daily dose of about 1,500 mg.

In certain embodiments, hydroxychloroquine sulfate is administered at a daily dose of about 2 mg/kg to about 25 mg/kg. In certain embodiments, hydroxychloroquine sulfate is administered at a daily dose of about 5 mg/kg to about 20 mg/kg. In certain embodiments, hydroxychloroquine sulfate is administered at a daily dose of about 6.5 mg/kg to about 19.5 mg/kg. In certain embodiments, hydroxychloroquine sulfate is administered at a daily dose of about 2.5 mg/kg. In certain embodiments, hydroxychloroquine sulfate is administered at a daily dose of about 3 mg/kg. In certain embodiments, hydroxychloroquine sulfate is administered at a daily dose of about 3.5 mg/kg. In certain embodiments, hydroxychloroquine sulfate is administered at a daily dose of about 4 mg/kg. In certain embodiments, hydroxychloroquine sulfate is administered at a daily dose of about 4.5 mg/kg. In certain embodiments, hydroxychloroquine sulfate is administered at a daily dose of about 5 mg/kg. In certain embodiments, hydroxychloroquine sulfate is administered at a daily dose of about 5.5 mg/kg. In certain embodiments, hydroxychloroquine sulfate is administered at a daily dose of about 6 mg/kg. In certain embodiments, hydroxychloroquine sulfate is administered at a daily dose of about 6.5 mg/kg. In certain embodiments, hydroxychloroquine sulfate is administered at a daily dose of about 7 mg/kg. In certain embodiments, hydroxychloroquine sulfate is administered at a daily dose of about 7.5 mg/kg. In certain embodiments, hydroxychloroquine sulfate is administered at a daily dose of about 8 mg/kg. In certain embodiments, hydroxychloroquine sulfate is administered at a daily dose of about 8.5 mg/kg. In certain embodiments, hydroxychloroquine sulfate is administered at a daily dose of about 9 mg/kg. In certain embodiments, hydroxychloroquine sulfate is administered at a daily dose of about 9.5 mg/kg. In certain embodiments, hydroxychloroquine sulfate is administered at a daily dose of about 10 mg/kg. In certain embodiments, hydroxychloroquine sulfate is administered at a daily dose of about 10.5 mg/kg. In certain embodiments, hydroxychloroquine sulfate is administered at a daily dose of about 11 mg/kg. In certain embodiments, hydroxychloroquine sulfate is administered at a daily dose of about 11.5 mg/kg. In certain embodiments, hydroxychloroquine sulfate is administered at a daily dose of about 12 mg/kg. In certain embodiments, hydroxychloroquine sulfate is administered at a daily dose of about 12.5 mg/kg. In certain embodiments, hydroxychloroquine sulfate is administered at a daily dose of about 13 mg/kg. In certain embodiments, hydroxychloroquine sulfate is administered at a daily dose of about 13.5 mg/kg. In certain embodiments, hydroxychloroquine sulfate is administered at a daily dose of about 14 mg/kg. In certain embodiments, hydroxychloroquine sulfate is administered at a daily dose of about 14.5 mg/kg. In certain embodiments, hydroxychloroquine sulfate is administered at a daily dose of about 15 mg/kg. In certain embodiments, hydroxychloroquine sulfate is administered at a daily dose of about 15.5 mg/kg. In certain embodiments, hydroxychloroquine sulfate is administered at a daily dose of about 16 mg/kg. In certain embodiments, hydroxychloroquine sulfate is administered at a daily dose of about 16.5 mg/kg. In certain embodiments, hydroxychloroquine sulfate is administered at a daily dose of about 17 mg/kg. In certain embodiments, hydroxychloroquine sulfate is administered at a daily dose of about 17.5 mg/kg. In certain embodiments, hydroxychloroquine sulfate is administered at a daily dose of about 18 mg/kg. In certain embodiments, hydroxychloroquine sulfate is administered at a daily dose of about 18.5 mg/kg. In certain embodiments, hydroxychloroquine sulfate is administered at a daily dose of about 19 mg/kg. In certain embodiments, hydroxychloroquine sulfate is administered at a daily dose of about 19.5 mg/kg. In certain embodiments, hydroxychloroquine sulfate is administered at a daily dose of about 20 mg/kg.

The daily dose of hydroxychloroquine sulfate may be administered as one dose or divided into two or more doses—e.g., b.i.d. In certain embodiments, the daily dose of hydroxychloroquine sulfate is administered as one dose. In certain embodiments, the daily dose of hydroxychloroquine sulfate is divided into two doses and administered b.i.d.

In certain embodiments, chloroquine phosphate is administered at a daily dose of about 50 mg to about 2000 mg, which is equivalent to about 30 mg to about 1200 mg chloroquine base. In certain embodiments, chloroquine phosphate is administered at a daily dose of about 150 mg to about 1800 mg. In certain embodiments, chloroquine phosphate is administered at a daily dose of about 250 mg to about 1500 mg. In certain embodiments, chloroquine phosphate is administered at a daily dose of about 500 mg to about 1500 mg. In certain embodiments, chloroquine phosphate is administered at a daily dose of about 500 mg to about 1000 mg. In certain embodiments, chloroquine phosphate is administered at a daily dose of about 1000 mg to about 1500 mg. In certain embodiments, chloroquine phosphate is administered at a daily dose of about 250 mg. In certain embodiments, chloroquine phosphate is administered at a daily dose of about 500 mg. In certain embodiments, chloroquine phosphate is administered at a daily dose of about 750 mg. In certain embodiments, chloroquine phosphate is administered at a daily dose of about 1000 mg. In certain embodiments, chloroquine phosphate is administered at a daily dose of about 1,250 mg. In certain embodiments, chloroquine phosphate is administered at a daily dose of about 1,500 mg. In certain embodiments, chloroquine phosphate is administered at a daily dose of about 1750 mg. In certain embodiments, chloroquine phosphate is administered at a daily dose of about 2000 mg. In certain embodiments, chloroquine phosphate is administered at a daily dose of about 2250 mg. In certain embodiments, chloroquine phosphate is administered at a daily dose of about 2500 mg.

In certain embodiments, chloroquine phosphate is administered at a daily dose of about 2 mg/kg to about 25 mg/kg. In certain embodiments, chloroquine phosphate is administered at a daily dose of about 5 mg/kg to about 20 mg/kg. In certain embodiments, chloroquine phosphate is administered at a daily dose of about 6.5 mg/kg to about 19.5 mg/kg. In certain embodiments, chloroquine phosphate is administered at a daily dose of about 2.5 mg/kg. In certain embodiments, chloroquine phosphate is administered at a daily dose of about 3 mg/kg. In certain embodiments, chloroquine phosphate is administered at a daily dose of about 3.5 mg/kg. In certain embodiments, chloroquine phosphate is administered at a daily dose of about 4 mg/kg. In certain embodiments, chloroquine phosphate is administered at a daily dose of about 4.5 mg/kg. In certain embodiments, chloroquine phosphate is administered at a daily dose of about 5 mg/kg. In certain embodiments, chloroquine phosphate is administered at a daily dose of about 5.5 mg/kg. In certain embodiments, chloroquine phosphate is administered at a daily dose of about 6 mg/kg. In certain embodiments, chloroquine phosphate is administered at a daily dose of about 6.5 mg/kg. In certain embodiments, chloroquine phosphate is administered at a daily dose of about 7 mg/kg. In certain embodiments, chloroquine phosphate is administered at a daily dose of about 7.5 mg/kg. In certain embodiments, chloroquine phosphate is administered at a daily dose of about 8 mg/kg. In certain embodiments, chloroquine phosphate is administered at a daily dose of about 8.5 mg/kg. In certain embodiments, chloroquine phosphate is administered at a daily dose of about 9 mg/kg. In certain embodiments, chloroquine phosphate is administered at a daily dose of about 9.5 mg/kg. In certain embodiments, chloroquine phosphate is administered at a daily dose of about 10 mg/kg. In certain embodiments, chloroquine phosphate is administered at a daily dose of about 10.5 mg/kg. In certain embodiments, chloroquine phosphate is administered at a daily dose of about 11 mg/kg. In certain embodiments, chloroquine phosphate is administered at a daily dose of about 11.5 mg/kg. In certain embodiments, chloroquine phosphate is administered at a daily dose of about 12 mg/kg. In certain embodiments, chloroquine phosphate is administered at a daily dose of about 12.5 mg/kg. In certain embodiments, chloroquine phosphate is administered at a daily dose of about 13 mg/kg. In certain embodiments, chloroquine phosphate is administered at a daily dose of about 13.5 mg/kg. In certain embodiments, chloroquine phosphate is administered at a daily dose of about 14 mg/kg. In certain embodiments, chloroquine phosphate is administered at a daily dose of about 14.5 mg/kg. In certain embodiments, chloroquine phosphate is administered at a daily dose of about 15 mg/kg. In certain embodiments, chloroquine phosphate is administered at a daily dose of about 15.5 mg/kg. In certain embodiments, chloroquine phosphate is administered at a daily dose of about 16 mg/kg. In certain embodiments, chloroquine phosphate is administered at a daily dose of about 16.5 mg/kg. In certain embodiments, chloroquine phosphate is administered at a daily dose of about 17 mg/kg. In certain embodiments, chloroquine phosphate is administered at a daily dose of about 17.5 mg/kg. In certain embodiments, chloroquine phosphate is administered at a daily dose of about 18 mg/kg. In certain embodiments, chloroquine phosphate is administered at a daily dose of about 18.5 mg/kg. In certain embodiments, chloroquine phosphate is administered at a daily dose of about 19 mg/kg. In certain embodiments, chloroquine phosphate is administered at a daily dose of about 19.5 mg/kg. In certain embodiments, chloroquine phosphate is administered at a daily dose of about 20 mg/kg.

The daily dose of chloroquine phosphate may be administered as one dose or divided into two or more doses—e.g., b.i.d. In certain embodiments, the daily dose of chloroquine phosphate is administered as one dose. In certain embodiments, the daily dose of chloroquine phosphate is divided into two doses and administered b.i.d.

In certain embodiments, cabozantinib is administered at a daily dose of about 1 mg to about 100 mg. In certain embodiments, cabozantinib is administered at a daily dose of about 5 mg to about 100 mg. In certain embodiments, cabozantinib is administered at a daily dose of about 5 mg to about 60 mg. In certain embodiments, cabozantinib is administered at a daily dose of about 10 mg to about 60 mg. In certain embodiments, cabozantinib is administered at a daily dose of about 15 mg to about 60 mg. In certain embodiments, cabozantinib is administered at a daily dose of about 20 mg to about 60 mg. In certain embodiments, cabozantinib is administered at a daily dose of about 5 mg. In certain embodiments, cabozantinib is administered at a daily dose of about 10 mg. In certain embodiments, cabozantinib is administered at a daily dose of about 15 mg. In certain embodiments, cabozantinib is administered at a daily dose of about 20 mg. In certain embodiments, cabozantinib is administered at a daily dose of about 25 mg. In certain embodiments, cabozantinib is administered at a daily dose of about 30 mg. In certain embodiments, cabozantinib is administered at a daily dose of about 35 mg. In certain embodiments, cabozantinib is administered at a daily dose of about 40 mg. In certain embodiments, cabozantinib is administered at a daily dose of about 45 mg. In certain embodiments, cabozantinib is administered at a daily dose of about 50 mg. In certain embodiments, cabozantinib is administered at a daily dose of about 55 mg. In certain embodiments, cabozantinib is administered at a daily dose of about 60 mg. Preferably, the cabozantinib is administered once daily. In certain embodiments, 20 mg cabozantinib is administered once daily. In certain embodiments, 40 mg cabozantinib is administered once daily. In certain embodiments, 60 mg cabozantinib is administered once daily. Preferably, cabozantinib is administered without food.

In certain embodiments, crizotinib is administered at a daily dose of about 50 mg to about 1,000 mg. In certain embodiments, crizotinib is administered at a daily dose of about 50 mg to about 500 mg. In certain embodiments, crizotinib is administered at a daily dose of about 100 mg to about 500 mg. In certain embodiments, crizotinib is administered at a daily dose of about 100 mg. In certain embodiments, crizotinib is administered at a daily dose of about 150 mg. In certain embodiments, crizotinib is administered at a daily dose of about 200 mg. In certain embodiments, crizotinib is administered at a daily dose of about 250 mg. In certain embodiments, crizotinib is administered at a daily dose of about 300 mg. In certain embodiments, crizotinib is administered at a daily dose of about 350 mg. In certain embodiments, crizotinib is administered at a daily dose of about 400 mg. In certain embodiments, crizotinib is administered at a daily dose of about 450 mg. In certain embodiments, crizotinib is administered at a daily dose of about 500 mg. In certain embodiments, the crizotinib is administered once daily. In certain embodiments, the daily dose of crizotinib is split and half the daily dose is administered twice daily. In certain embodiments, 200 mg crizotinib is administered twice daily. In certain embodiments, 250 mg crizotinib is administered twice daily.

In certain embodiments, the glutaminase inhibitor (e.g., telaglenastat) is administered at a daily dose of from about 50 mg to about 2000 mg. In certain embodiments, the glutaminase inhibitor (e.g., telaglenastat) is administered at a daily dose of from about 50 mg to 150 mg, about 150 mg to about 250 mg, about 250 mg to about 350 mg, about 350 mg to about 450 mg, about 450 mg to about 550 mg, about 550 mg to about 650 mg, about 650 mg to about 750 mg, about 750 mg to about 850 mg, about 850 mg to about 950 mg, about 950 mg to about 1050 mg, about 1050 mg to about 1150 mg, about 1150 mg to about 1250 mg, about 1250 mg to about 1350 mg, about 1350 mg to about 1450 mg, about 1450 mg to about 1550 mg, about 1550 mg to about 1650 mg, about 1650 mg to about 1750 mg, about 1750 mg to about 1850 mg, or about 1850 mg to about 1950 mg. In certain embodiments, the glutaminase inhibitor (e.g., telaglenastat) is administered at a daily dose of from about 600 mg to about 800 mg. In certain embodiments, the glutaminase inhibitor (e.g., telaglenastat) is administered at a daily dose of from about 1200 mg to about 1600 mg. In certain embodiments, the glutaminase inhibitor (e.g., telaglenastat) is administered at a daily dose of about 600 mg. In certain embodiments, the glutaminase inhibitor (e.g., telaglenastat) is administered at a daily dose of about 800 mg. In certain embodiments, the glutaminase inhibitor (e.g., telaglenastat) is administered at a daily dose of about 1200 mg. In certain embodiments, the glutaminase inhibitor (e.g., telaglenastat) is administered at a daily dose of about 1600 mg.

The daily dose of glutaminase inhibitor may be administered as one dose or divided into two or more doses—e.g., b.i.d. In certain embodiments, the daily dose of glutaminase inhibitor is administered as one dose. In certain embodiments, the daily dose of glutaminase inhibitor is divided into two doses and administered b.i.d. In certain embodiments, the daily dose of telaglenastat is administered as one dose. In certain embodiments, the daily dose of telaglenastat is divided into two doses and administered b.i.d.

In certain embodiments, the dose of telaglenastat is administered orally. In certain embodiments, the dose of telaglenastat is administered orally without food. In certain embodiments, the dose of telaglenastat is administered orally with food.

For simplicity, the fatty acid oxidation inhibitor, tyrosine kinase inhibitor, glutaminase inhibitor, and/or glycolysis inhibitor may be administered pursuant to a treatment cycle that is the same length as each treatment cycle for devimistat (e.g., 2 weeks, three weeks, four weeks, etc.). Like the cycle for devimistat, the fatty acid oxidation inhibitor, tyrosine kinase inhibitor, glutaminase inhibitor, and/or glycolysis inhibitor cycles may include days in which a dose of fatty acid oxidation inhibitor, tyrosine kinase inhibitor, glutaminase inhibitor, and/or glycolysis inhibitor is administered and days in which a dose of fatty acid oxidation inhibitor, tyrosine kinase inhibitor, glutaminase inhibitor, and/or glycolysis inhibitor is not administered. For example, the fatty acid oxidation inhibitor, tyrosine kinase inhibitor, glutaminase inhibitor, and/or glycolysis inhibitor may be administered pursuant to a schedule in which fatty acid oxidation inhibitor, tyrosine kinase inhibitor, glutaminase inhibitor, and/or glycolysis inhibitor is administered on the same days that devimistat is administered, and is not administered on the days devimistat is not administered. Alternatively, the fatty acid oxidation inhibitor, tyrosine kinase inhibitor, glutaminase inhibitor, and/or glycolysis inhibitor may be administered on some but not all days in which devimistat is administered, and/or may be administered on some but not all days on which devimistat is not administered. In certain embodiments, the fatty acid oxidation inhibitor, tyrosine kinase inhibitor, glutaminase inhibitor, and/or glycolysis inhibitor may be administered on each day of the cycle. When two or all three of a fatty acid oxidation inhibitor, tyrosine kinase inhibitor, glutaminase inhibitor, and glycolysis inhibitor are administered, they may be administered on the same or different days.

In certain embodiments, the dosing cycle is repeated at least once. In certain embodiments, the method of the present invention comprises treatment with two cycles or more. In certain embodiments, the method of the present invention comprises treatment with three cycles or more. In certain embodiments, the method of the present invention comprises treatment with four cycles or more. In certain embodiments, the method of the present invention comprises treatment with five cycles or more. In certain embodiments, the method of the present invention comprises treatment with six cycles or more. In certain embodiments, the method of the present invention comprises treatment with seven cycles or more. In certain embodiments, the method of the present invention comprises treatment with eight cycles or more. In certain embodiments, the method of the present invention comprises treatment with nine cycles or more. In certain embodiments, the method of the present invention comprises treatment with ten cycles or more. In certain embodiments, the method of the present invention comprises regular treatment with devimistat and a fatty acid oxidation inhibitor, tyrosine kinase inhibitor, glutaminase inhibitor, and/or glycolysis inhibitor, including on a daily or weekly basis, for an extended period of time, such as at least one month, six months, one year, two years, three years, or longer. In certain embodiments, treatment with a fatty acid oxidation inhibitor, tyrosine kinase inhibitor, glutaminase inhibitor, and/or glycolysis inhibitor may continue after treatment with devimistat ceases. In certain embodiments, treatment with devimistat may continue after treatment with a fatty acid oxidation inhibitor, tyrosine kinase inhibitor, glutaminase inhibitor, and/or glycolysis inhibitor ceases.

### Patients for Treatment

The therapeutic methods may be further characterized according to the patient to be treated. In the present invention, the patient is a human being. In certain embodiments, the patient is an adult. In certain embodiments, the patient is an adult at least 50 years of age. In certain embodiments, the patient is an adult at least 60 years of age. In certain embodiments, the patient is a child.

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial of at least 10 patients with cancer, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II or phase III clinical trial comprises at least 15 patients. More preferably, the phase II or phase III clinical trial comprises at least 20 patients. More preferably, the phase II or phase III clinical trial comprises at least 25 patients. More preferably, the phase II or phase III clinical trial comprises at least 50 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, the method of the present invention provides an overall response rate of at least about 20% in patients. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. In certain embodiments, the overall response rate, duration of response, and progression-free survival mentioned above are measured in a phase II clinical trial. In certain embodiments, the overall response rate, duration of response, and progression-free survival mentioned above are measured in a phase III clinical trial.

### EQUIVALENTS

The description above describes multiple aspects and embodiments of the invention, including therapeutic applications, treatment methods, and pharmaceutical compositions. The patent application specifically contemplates all combinations and permutations of the aspects and embodiments.

### III. Examples

The invention now being generally described, will be more readily understood by reference to the following examples, which are included merely for purposes of illustration of certain aspects and embodiments of the present invention, and are not intended to limit the invention.

### Methods

### Cell Culture

AsPC1, PANC1, H460, BxPC3, A549, and PC-3 cells were purchased from the American Type Culture Collection (Manassas, VA). PANC1, H460, BxPC3, and PC-3 were cultured in Roswell Park Memorial Institute (RPMI)-1640 medium and AsPC1 in Dulbecco's Modified Eagle Medium (DMEM), both media supplemented with 10% fetal bovine serum, 100 units/ml penicillin and 100 μg/ml streptomycin (ThermoFisher, Waltham, MA). For the last passage before metabolic analysis, all cell lines were cultured in parallel in RPMI. Cells were incubated at 37° C. in a humidified incubator with 5% CO₂. We avoid exposing tumor cell lines to heat-inactivated serum to maintain stable metabolic behavior.

The two PDAC lines of central focus, PANC1 and AsPC1, both resemble the majority genotype of pancreatic cancers in having activated KRAS and mutant p53 (Deer, E. L. et al., “Phenotype and Genotype of Pancreatic Cancer Cell Lines,” Pancreas, 2010, 39(4), 425-435). Moreover, CDKN2A/p16 is epigenetically down-regulated in both cell lines (Pan, F. P. et al., “Emodin enhances the demethylation by 5-Aza-CdR of pancreatic cancer cell tumor-suppressor genes P16, RASSF1A and ppENK,” Oncology Reports, 2016, 35(4), 1941-1949; Hong, L. et al., “The interaction between miR-148a and DNMT1 suppresses cell migration and invasion by reactivating tumor suppressor genes in pancreatic cancer,” Oncology Reports, 2018, 40(5), 2916-2925). The devimistat-resistant member of this pair, AsPC1, was isolated from a post-treatment metastasis (Chen W H et al., “Human pancreatic adenocarcinoma—in vitro and in vivo morphology of a new tumor line established from ascites,” In Vitro-Journal of the Tissue Culture Association, 1982, 18, 24-34); whereas devimistat-sensitive PANC1 was isolated from an untreated primary tumor (Lieber M et al., “Establishment of a continuous tumor-cell line (PANC1) from a human carcinoma of exocrine pancreas,” Int. J. Can., 1975, 15, 741-747; Deer, et al., 2010). AsPC1 displays SMAD4 loss, as do the majority of clinical PDACs (whereas PANC1 retains SMAD4 expression; Chen, Y. W. et al., “SMAD4 Loss triggers the phenotypic changes of pancreatic ductal adenocarcinoma cells,” Bmc Cancer, 2014, 14). Collectively, these features are consistent with AsPC1 being a useful model for difficult-to-treat clinical PDAC cases more generally.

### Adherent Carcinoma Cell Treatment Under Low Nutrient Level Conditions

Cells were plated at 80% confluency in complete RPMI in black, clear bottom 96-well plates or 35-mm dishes for ATP, protein and glycogen analysis, or glass coverslips for fluorescence visualization. To create low nutritional conditions, cells were seeded in complete media, after 24 hours cells were transferred to serum free RPMI for 18-24 hours. Prior to drug treatment, cells were incubated in a nutrient-free carbonate buffered salt solution (CBS2) for 3 hours. Drug treatments were then carried out in CBS2 with addition of nutrient sources and/or chemical agents as described in the Examples and Figures. CBS2 formulation is shown in the following table.

| CBS2 (modified Earle's | | | |
|---|---|---|---|
| balanced salt solution) | M.W. | mg/L | mM |
| Calcium Chloride (CaCl₂) (anhydride) | 111 | 100 | 0.9009 |
| Magnesium Sulfate (MgSO₄•7H₂O) | 246 | 200 | 0.8130 |
| Potassium Chloride (KCl) | 75 | 400 | 5.3333 |
| Sodium Bicarbonate (NaHCO₃) | 84 | 2200 | 26.1905 |
| Sodium Chloride (NaCl) | 58 | 6800 | 117.2414 |
| Sodium Phosphate monobasic | 138 | 140 | 1.0145 |
| (NaH₂PO₄•H₂O) | | | |
| Phenol Red | 398 | 2.5 | 0.0063 |

### Acute ATP Measurements and Cell Survival ATP Measurements

LOGIC: High throughput, plate reader assays for cell death essentially always ultimately depend on metabolic activity of cells to identify viability, directly or indirectly. Thus, strong inhibition of metabolic activity by anti-metabolism agents like those described herein can confound cell death analysis by such assays. To overcome this limitation, the inventors took advantage of the fact that the agents used herein ultimately inhibit mitochondrial metabolism. The inventors thus used a recovery-based assay wherein cells are treated with anti-metabolic agents under conditions where mitochondrial metabolism is (or becomes) limiting on viability. After experimental treatment, drug and media are removed and cells are allowed to recover in serum-free RPMI. RPMI provides high levels of glucose in support of glycolytic ATP generation (independently of mitochondrial function), allowing viable cells to recover ATP levels that are ultimately measured in the plate assay to score cell viability. Under these conditions, cells that are committed to execution of cell death by the experimental treatment score as dead and cells that have not committed to cell death recover ATP levels and score as viable.

Specifically, after exposure to the treatments indicated, cells are allowed to recover (17-28 hours) in RPMI without serum. Survival is assessed by conventional luminescence measurement of ATP levels after this recovery.

The inventors have validated this assay extensively in multiple independent ways including FACS analysis and video microscopy (Zachar, et al., “Non-redox-active lipoate derivatives disrupt cancer cell mitochondrial metabolism and are potent anticancer agents in vivo,” J. Mol. Med. 2011, 89, 1137-1148). We have also validated this assay by comparing hemocytometer and ATP cell survival measures. Moreover, the inventors commonly spot-checked recovery/ATP assays of cell death by direct microscopy.

TECHNICAL DETAILS: Immediately after drug treatments, acute ATP levels of cells in black, clear bottom 96-well plates were measured using the CellTiter-Glo® Luminescent Cell Viability Assay (catalog #G775B) following Promega (Madison, WI) protocols. To assess cell viability, after drug treatments, drug/agent-containing medium was replaced with serum-free RPMI and cells were incubated for an additional 17-24 hours to allow recovery of viable cells, without additional cell division. After this recovery period ATP levels were measured using the CellTiter-Glo® Luminescent Cell Viability Assay as described above.

### Steady State Metabolite Levels Determination

The steady state metabolomics analysis in Example 1C used established assays and procedures in collaboration with Metabolon, Inc. Other results from this study were originally described and reported in Stuart et al., 2014. In brief, samples were extracted and split into equal parts for analysis on the gas chromatography-mass spectrometry and liquid chromatography-tandem mass spectrometry platforms (Evans, A. M. et al., “Integrated, nontargeted ultrahigh performance liquid chromatography/electrospray ionization tandem mass spectrometry platform for the identification and relative quantification of the small-molecule complement of biological systems,” Anal. Chem. 2009, 81, 6656-6667). Proprietary software was used to match ions to an in-house library of standards for metabolite identification and for metabolite quantitation by peak area integration (DeHaven, C. D. et al., “Organization of GC/MS and LC/MS metabolomics data into chemical libraries,” J. Cheminform. 2010, 2, 9). Extracts were prepared according to Metabolon's (Durham, NC, USA) standard methanol-based extraction protocol (Evans, A. M. et al., 2009). Samples were analyzed on a Thermo-Finnigan Trace DSQ fast-scanning single-quadrupole mass spectrometer (Waltham, MA) using electron impact ionization.

Cell pellet and media preparations. Cell preparation was as follows. 3.5×10⁶ BxPC3 pancreatic cancer cells were plated in 10 cm plates in RMPI medium supplemented with 10% FBS and 100 U/mL penicillin and 100 μg/mL streptomycin. Cells were allowed to grow for two days reaching 90-95% confluence. At this time, the medium was replaced with fresh medium containing solvent (0.075% dimethylformamide) or 240 μM devimistat in solvent and treated for the times indicated in the Examples and Figures. At the end of treatment, plates were placed on ice, medium was removed and retained for analysis. Cells were washed with 3 mLs of ice-cold non-supplemented RMPI medium. 3 mLs of fresh ice-cold non-supplemented medium was added to the plates, cells were scraped and transferred into 15 mL semiconical tubes and centrifuged at 800×g for 3 minutes. Supernatant was removed, samples were spun for an additional 30 seconds and all residual medium was removed. Cell pellets were flash frozen in liquid nitrogen and stored at −80° C. until shipment to Metabolon for biochemical profiling.

Global untargeted biochemical profiling. The LC/MS portion of the platform was based on a Waters ACQUITY UPLC and a Thermo-Finnigan LTQ mass spectrometer, which consisted of an electrospray ionization (ESI) source and linear ion-trap (LIT) mass analyzer. The sample media or cell pellet extract was split into two aliquots, dried, then reconstituted in acidic or basic LC-compatible solvents, each of which contained 11 injection standards at fixed concentrations. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% Formic acid, while the basic extracts, which also used water/methanol, contained 6.5 mM ammonium bicarbonate. The MS analysis alternated between MS and data-dependent MS (DeHaven, C. D. et al., “Organization of GC/MS and LC/MS metabolomics data into chemical libraries,” J. Cheminform. 2010, 2, 9) scans using dynamic exclusion. The inventors performed chromatographic separation, followed by full-scan mass spectroscopy, to record and quantify all detectable ions presented in the samples. The inventors identified metabolites with known chemical structure by matching the ions' chromatographic retention index and mass spectral fragmentation signatures with reference library entries created from authentic standard metabolites under the identical analytical procedure as the experimental samples.

An aliquot of each experimental media sample was taken then pooled for the creation of “Client Matrix” (CMTRX) samples. These CMTRX samples were injected throughout the platform run and served as technical replicates, allowing variability in the quantitation of all consistently detected endogenous biochemicals to be determined and overall process variability and platform performance to be monitored.

Statistical Analysis. Data normalization was performed to correct variation resulting from instrument inter-day tuning differences. Each compound was corrected in run-day blocks by registering the medians to equal one (1.00) and normalizing each data point proportionately. Missing values (if any) were assumed to be below the level of detection for that biochemical with the instrumentation used and were imputed with the observed minimum for that particular biochemical. After normalization and imputation, the data were log-transformed. The inventors then performed t tests to compare the treatment conditions as well as the time points across treatments. Multiple comparisons were accounted for with the false discovery (FDR) rate method, and each FDR was estimated by q-values.

### 2-Deoxyglucose Uptake

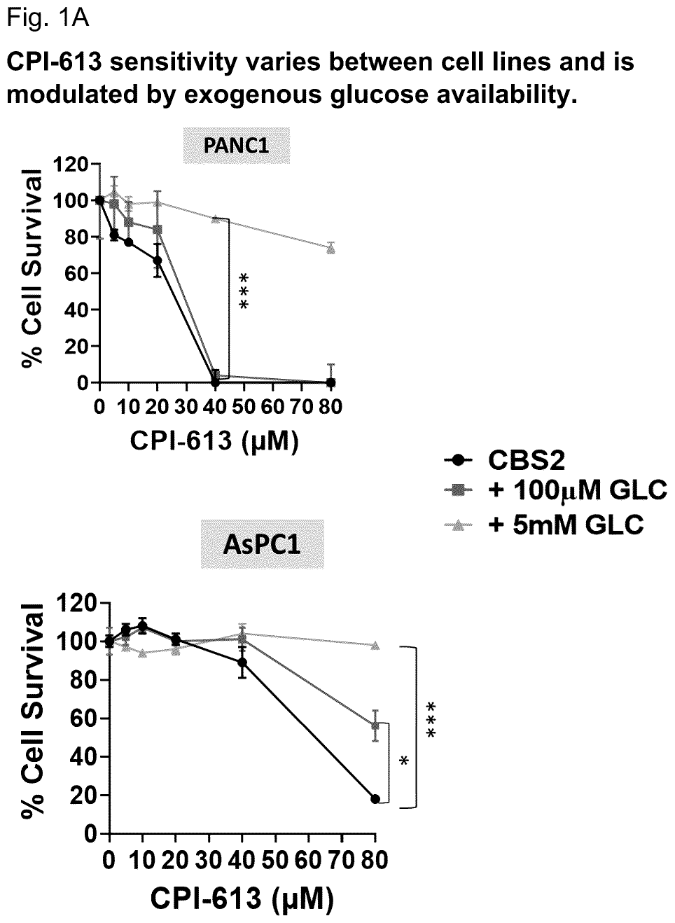

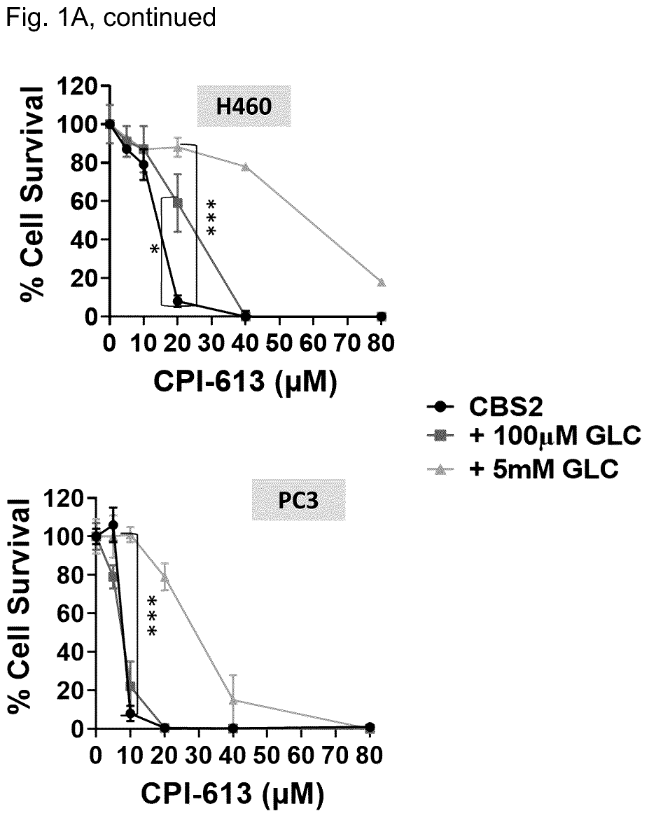

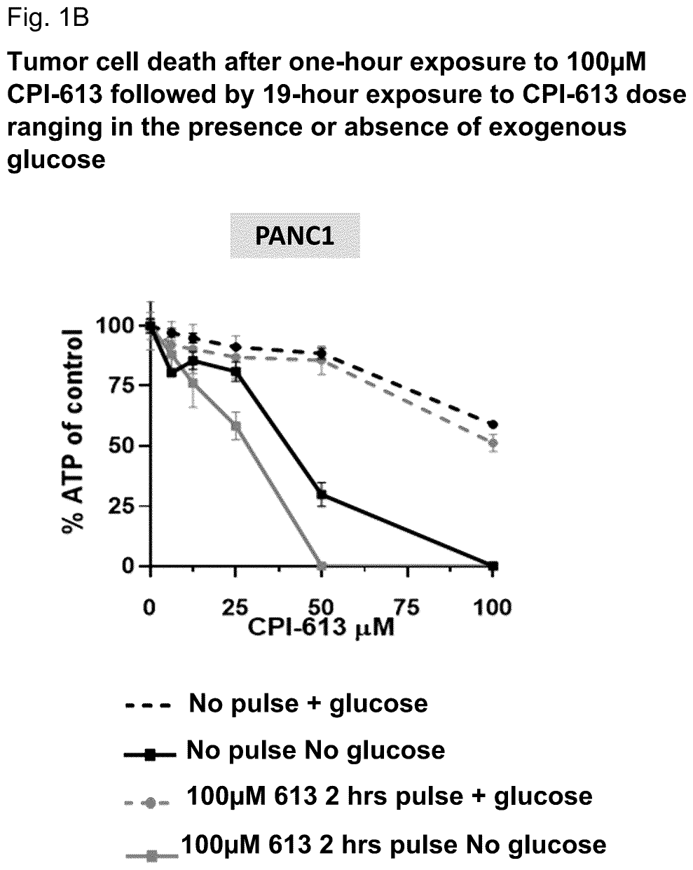

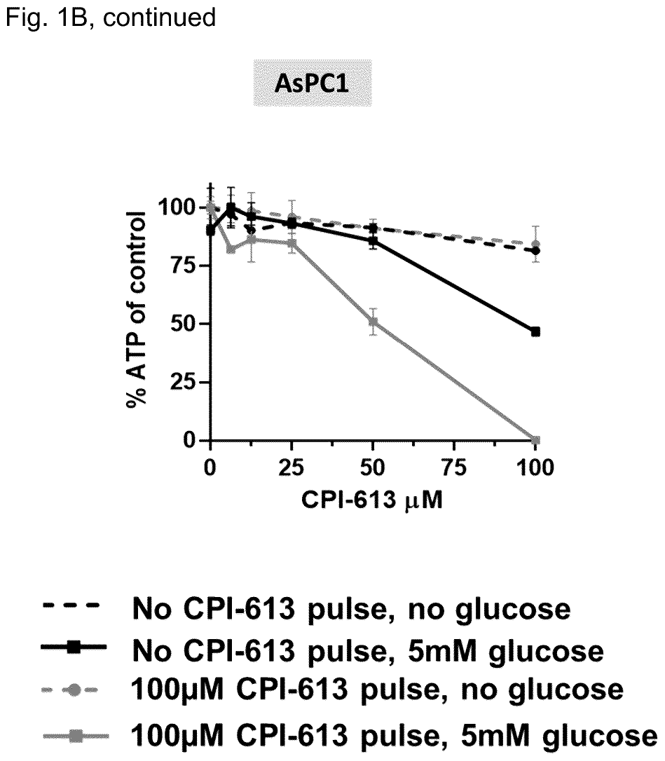

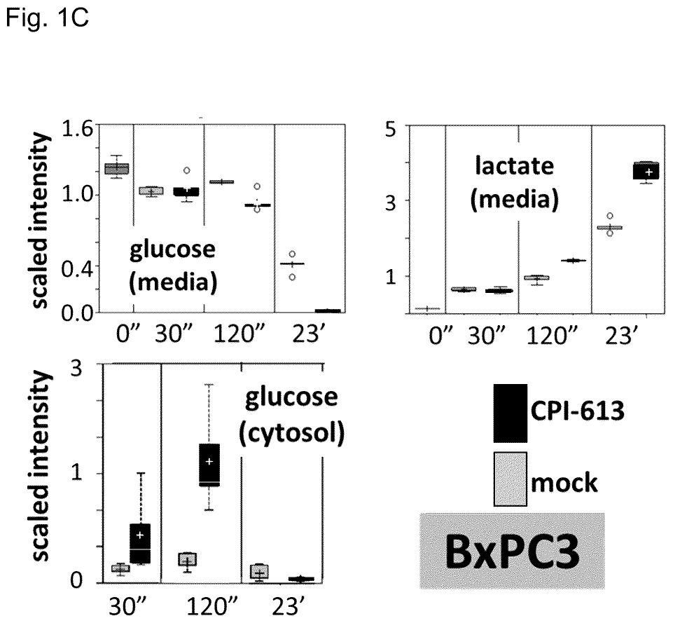

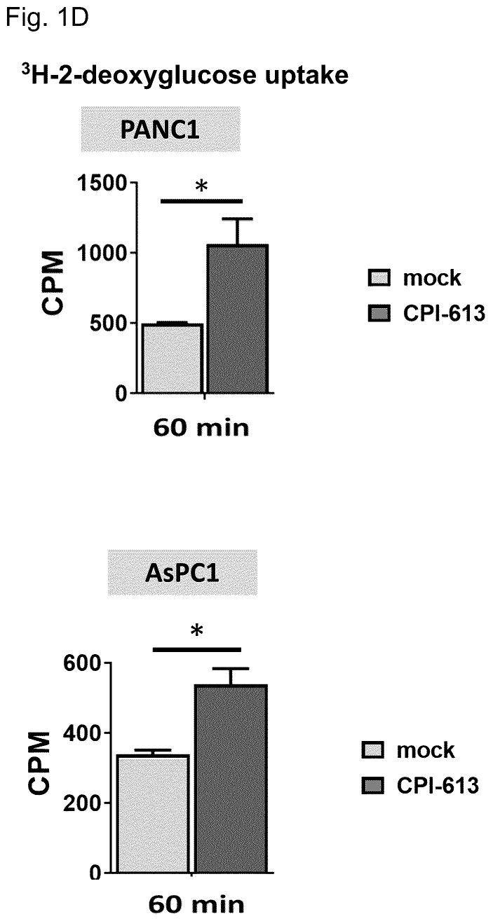

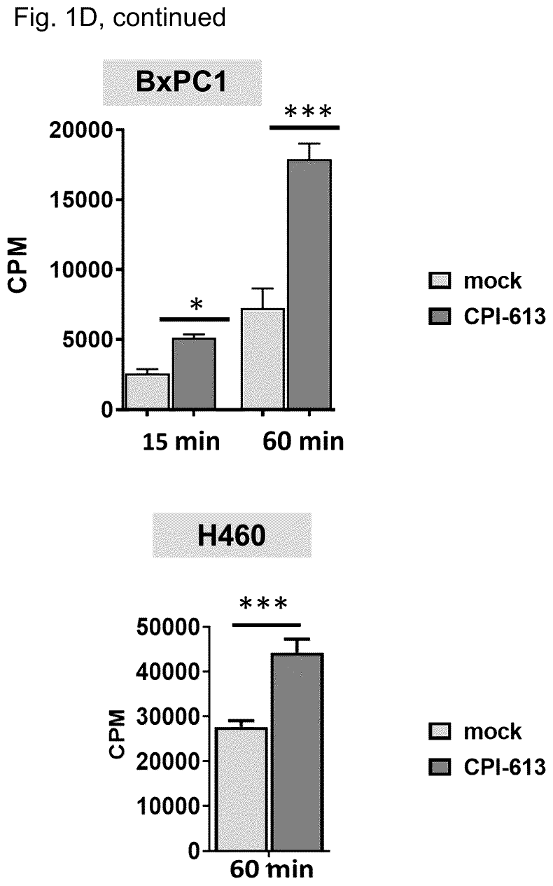

Cells were treated in complete RPMI with devimistat (240 μM) for a total of 2 hours, with a pulse of 1,2-³H 2-deoxyglucose at the end of this treatment for the times indicated in Example 1D and FIG. 1D. Cells were harvested, washed, and counted to asses uptake. More particularly, seeding, drug treatment, and 2DG uptake were all performed in RPMI medium supplemented with 10% FBS for BXPC3, H460 and PANC1 cells and DMEM+10% FBS for AsPC1. All treatments were done in triplicate. BxPC3 cells were seeded at 500,000 cells per 35-mm dish; H460, PANC1, and AsPC1 cells were plated in 6 well plates at 300,000 cells per well for PANC1 and at 500,000 cells per well for AsPC1 and H460. Following a 48-hour incubation, the seeding medium was replaced with fresh medium and incubated for 2 hours. At this time, solvent (control) or devimistat (240 μM) were added to the medium and incubated for 2 hours. One hour after the addition of solvent or devimistat, 0.5 μCi of 1,2-³H 2-deoxyglucose (Moravek Biochemicals) was added to the medium of AsPC1, PANC1, BxPC3 and H460 cells, 0.5 μCi of 1,2-³H 2-deoxyglucose was added to the medium 1 hour and 45 minutes after the addition of solvent or devimistat. To minimize exposure of cells to extreme surface tension, all additions of solvent, devimistat, and 2-deoxyglucose were done by removing ½ of medium and replacing with equivalent media volume containing appropriate concentration of agents.

At the end of the 2 hour drug treatment, plates were placed on ice, medium removed and plates were washed three times with ice cold medium without FBS. 300 μL of 0.5% triton in PBS was added to each plate/well and plates were incubated at room temperature for 10 minutes. Lysates were transferred into scintillation vials containing 3 mL of scintillation cocktail and counted.

### Staining of Lipid Droplets

Approximately 500,000 cells were seeded in 35-mm culture dishes containing coverslips. Following treatment, coverslips were washed in ice cold PBS, fixed for 18 hrs at 4° C. in 5% paraformaldehyde then exposed to the neutral lipid stain BODIPY 493/503 (Molecular Probes) at a concentration of 50 μg/mL for 2 hours at room temperature. Coverslips were mounted in ProLong Diamond plus DAPI mounting solution (Molecular Probes) and captured on an Axiovert 200M (Zeiss) deconvolution microscope using AxioVision software (version 4.5) at a fixed exposure time.

### Determination of Glycogen Content

Glycogen was quantified using the Glycogen Assay Kit II (colorimetric) from AbCam (ab169558) according to manufacturer's instructions. Briefly, approximately 2×10⁶ cells were washed with ice-cold PBS and collected via scraping in 300 μL ice-cold ddH₂O. Cells were immediately sonicated on ice for 5× is pulses with a probe sonicator (Misonix Inc, model XL2000), then heated to 80° C. for 10 min. Samples were stored O/N at −20° C., then thawed, centrifuged at 12,000×g for 10 min and the supernatants collected for glycogen hydrolysis and quantification according to manufacturer's instructions.

### Xenograft, Tumor Growth Inhibition Studies

Female, 6-week-old, CD1-nu/nu mice were inoculated with 2.5 million PANC-1 cell in 100 μL of HBSS or 5 million AsPC-1 cells in HBSS. Treatment commenced when tumors reached 100 mm³ and lasted for the times indicated in the Examples and Figures. Mice were treated three times per week (MWF) with vehicle or drugs as indicated in the Examples and Figures. Tumors were measured once per week. Administration route was oral gavage for crizotinib and intraperitoneal for devimistat and thioridazine. Use of Matrigel for tumor inoculation was avoided in TGI experiments with this class of agents.

### Thioridazine (TZ) and Etomoxir (ETX) Inhibition of CO₂ Release

Radioactive CO₂ released from AsPC1 cells was captured after treatment with TZ, ETX, or mock control for 1 hour, followed by 30 minute pulse 0.1 μCi of 1-¹⁴C labeled oleic acid. More particularly, AsPC-1 cells were seeded in 12 well plates at 500,000 cells per well in DMEM medium supplemented with 10% FBS and incubated for 48 hours. At this time, the seeding medium was replaced with DMEM without serum and incubated 20 hrs. The serum free medium was replaced with 700 μL CBS2 supplemented with 40 μM oleic acid (OA) and 50 μM CP91149 (glycogen phosphorylase inhibitor, GPi) and cells were incubated for 3 hours. After three-hour incubation, thioridazine or etomoxir were added at the concentration indicated in the Examples and Figures and samples were incubated for an additional 1 hour. 0.1 μCi of 1-¹⁴C labeled oleic acid (Moravek Biochemicals) was then added to each well and incubated for an additional 30 minutes, resulting in a 30-minute labeling pulse. The samples were terminated by addition of 1M perchloric acid to a final concentration of 150 mM. In addition to killing the cells and terminating biochemical reactions, the acidification of the medium results in the release of CO₂ from sodium carbonate in the medium. The released CO₂ is captured in phenyl ethyl amine saturated filters placed on top of each well. Collection of released CO₂ is done for ˜20 hrs, at which time the filters are counted.

### Morpholino Antisense Oligo Knockdown of Acox1

Westerns: Plate 50,000 AsPC-1 cells/well in 24 well plates in complete DMEM in peripheral wells.

Morpholino treatment: 24 hrs post plating, replace seeding medium with 0.5 ml serum free DMEM+2.5 μM total of Acox1 Morpholino (MO) mixture or Vivo-Control. Incubate for 6 hrs. Replace morpholino solutions with complete DMEM.

Cells were harvested 48 hrs later and analyzed for protein levels via Western blot using appropriate antibodies (Acox1—AbCam ab184032, beta-actin—Sigma A5060)(Example 3N). Morpholinos were obtained from Gene Tools, LLC, Philomath, OR

MO1 sequence:
5′-CTGGCAGCGAAGTAACGACCGACC,
MO2 sequence:
5′-GCAGTGACAATCTAAATCCGCAGCT,
Vivo-Control sequence:
5′-CCTCTTACCTCAGTTACAATTTATA

Cell death assay: Plate 15,000 AsPC-1 cells/well in 96 well plates in complete DMEM.

Morpholino treatment: 24h post plating, replace seeding medium with 50 μl serum free DMEM+2.5 μM total of Acox1 Morpholino (MO) mixture or Vivo-Control. Incubate for 6 hrs. Replace morpholino solutions with complete DMEM.

Assessment of Acox1 knockdown on cell survival: 2 days post morpholino treatment, complete DMEM was replaced with DMEM without serum and incubated for 3.5 hrs. DMEM without serum was then replaced with CBS2 and incubated for 21 hrs followed by recovery in DMEM without serum for 24 hrs and cell viability was assayed with CellTiterGLO (Promega).

### Example 1. Exogenous Glucose Availability and Response to Devimistat Treatment

A. Exogenous glucose protects from devimistat-induced cell death. Four different cell lines were exposed for 15 hours (PANC1, AsPC1, H460) or 8 hours (PC3) to the indicated range of devimistat doses in the absence of exogenous nutrients (in carbonate buffered balanced salts solution, CBS2) after 3 hours of preincubation in CBS2 (for cell adaptation and to equilibrate soluble nutrient pools). Glucose (capable of supporting glycolytic ATP generation) was added to CBS2 as indicated in FIG. 1A. Cells were exposed to the treatments indicated, followed by recovery (24 hours) in RPMI without serum (providing nutrient support for viable cell recovery, without serum factors for cell division). Survival is assessed by conventional luminescence measurement of ATP levels after this recovery. See the Methods section above for a detailed description of this assay and its validation. The relatively large quantitative differences in devimistat sensitivity between cell lines under exogenous nutrient deprivation are characteristic of each line (see examples below), with small experiment-to-experiment variation. The results are presented in FIG. 1A. Statistical confidence (p-values) for CBS2 versus CBS2+glucose comparisons for boxed point pairs are indicated. The results show that devimistat sensitivity is strongly influenced by extracellular glucose availability.

B. Transient exposure to elevated devimistat levels sensitizes to subsequent prolonged exposure to lower levels. Tumor cell lines are exposed to a two-hour pulse of 100 M devimistat (or mock), followed by a 19-hour exposure to the devimistat concentrations indicated in FIG. 1B in CBS2 without exogenous nutrients or with 5 mM glucose. This transient, high-dose exposure produces little or no commitment to cell death alone; however, it sensitizes cells modestly, but significantly to subsequent prolonged, low-dose exposure. The results are presented in FIG. 1B. The results show that devimistat sensitivity is influenced by extracellular glucose availability.

C. Steady state metabolomics analysis indicates devimistat stimulation of glucose uptake (from media and into cell pellets; cytosol) and secretion of lactate. BxPC3 PDAC cells were treated with 240 μM devimistat in complete RPMI for the times shown in FIG. 1C (30 minutes, 120 minutes, 23 hours; 0 minutes indicates measurements on virgin medium). This steady state metabolomics analysis used established assays and procedures in collaboration with Metabolon, Inc and was originally described and reported in Stuart, S. D. et al., “A strategically designed small molecule attacks alpha-ketoglutarate dehydrogenase in tumor cells through a redox process.” Cancer & Metabolism 2014, 2, 4. The results are presented in FIG. 1C; measured values are expressed as scaled intensity.

D. Pulse analysis of ³H-2-deoxyglucose (2DG) uptake by tumor cell lines. ˜8×10⁵ cells were treated with 240 μM devimistat or solvent control in complete RPMI for two hours and pulse labeled by addition of 0.5 μCi of ³H-2DG for the final 0.25 or 1 hour of treatment as indicated in FIG. 1D. Cells were washed in ice-cold RPMI without serum, lysed, and counted (see Methods, above). Results are presented in FIG. 1D. This assay confirms the devimistat-accelerated glucose uptake in BxPC3 cells indicated by steady state metabolomics and further demonstrates similar effects in PANC1 and AsPC1 PDAC lines and the H460 NSCLC line.

### Example 2. Endogenous Nutrient Stores and Response to Devimistat Treatment

A. Two PDAC cell lines show large, consistent differences in sensitivity to devimistat under exogenous nutrient deprivation. PANC1 and AsPC1 cells were treated in CBS2 at the devimistat doses and for the times indicated in FIG. 2A, followed by recovery (19-21 hours) to assess commitment to cell death in three independent experiments (see Methods and Example 1A). Results are presented FIG. 2A.

B. Xenograft tumor growth inhibition (TGi) assessment of in vivo devimistat sensitivity of PANC1 and AsPC1 tumors. Tumors were inoculated subcutaneously on the flank and drug was administered intraperitoneally (IP) three days per week (MWF) (see Methods, above). Tumor volume was measured by caliper assessment of tumor radius and calculation of volume (see Methods, above). Results are presented in FIG. 2B. Nominal Cmax indicates the serum drug concentration that would be achieved if the entire IP dose moved immediately and exclusively into the serum. Actual Cmax experienced by tumor will generally be significantly lower than this value.

C. Comparison of endogenous glycogen stores and their devimistat-induced depletion in PANC1 and AsPC1 cells. Cells were treated in CBS2 for 3 hours with 30 μM devimistat or mock treated (FIG. 2C, left), or treated for 3 hours with the indicated devimistat doses (FIG. 2C, right). Glycogen quantitation is described in Methods, above. ** indicates p<0.01. **** indicates p<0.0001.

D. Comparison of lipid store staining in PANC1 (sensitive) and AsPC1 (resistant) cell lines. Lipid fluorescence staining and microscopy was carried out with cells treated as the control sample in Example 2C (see Methods, above). Results are presented in FIG. 2D. At top is both the green lipid signal and DAPI blue nuclear signal in color; at bottom is the lipid signal alone in black and white.

E. Response of AsPC1 lipid store staining to devimistat treatment. Lipid staining fluorescence microscopy was carried out under the conditions in Example 2C except treatment time was 21 hours. Result are presented in FIG. 2E (additional treatment components are indicated in superposed white text).

F. Time-dependent effects of devimistat exposure on metabolism (acute ATP levels) and commitment to cell death (ATP levels after rich-medium recovery). Cells were exposed to the indicated concentrations of devimistat (horizontal axis) for the times indicated (FIG. 2F, top left and top right) as in Example 1A. ATP levels were measured in duplicate plates either immediately for acute effects on ATP levels (FIG. 2F, top left) or after 20 hours of recovery (as in Example 1A) to measure survival versus induction of commitment to cell death (FIG. 2F, top right). Raw data are shown in all of the upper panels of FIG. 2F except the leftmost. To ease visualization of the relevant results in this panel (FIG. 2F, upper left), the inventors internally normalized each time set to its drug-free control for this PANC1 acute panel, with each control being normalized to the mean of the drug-free samples. The chevrons at the left of this panel (FIG. 2F, upper left) indicate the non-normalized ATP levels in the absence of devimistat. Notice that the 1 and 2 hour treatment times have negligible effects on ATP levels in the absence of drug. In AsPC1 cells time (in the absence of devimistat) has negligible effects on ATP levels. In contrast, in the sensitive PANC1 line there is modest but significant variation in acute ATP levels as a function of time in the absence of devimistat (FIG. 2F, upper left plot). Relevant statistical significance levels (p values) are as follows: PANC1 (40 μM; acute) 1 hr/8 hrs (<0.05); PANC1 (40 μM; cell death) 1 hr/8 hrs (<0.0005); AsPC1 (40 μM; acute) 1 hr/2 hr (<0.007), 1 hr/4 hr (<0.001), 1 hr/8 hr (<8×10⁻⁶); AsPC1 (40 μM; cell) 1 hr/4 hr (<0.003).

In an analogous fashion, NSCLC H460 cells were exposed to the indicated doses of devimistat for the indicated times (FIG. 2F, bottom left and bottom right). ATP levels were measured in duplicate plates for acute effects on ATP levels (FIG. 2F, bottom left) and induction of commitment to cell death, after recovery incubation (FIG. 2F, bottom right) (see Methods, above).

G. Effects of pre-feeding PANC1 and AsPC1 cells with glucose and/or oleic acid on resistance to subsequent devimistat-induced acute ATP loss and commitment to cell death in the absence of other exogenous nutrients. Cells were pre-fed for 5 hours with glucose and/or oleic acid. Exogenous nutrient containing medium was removed, cells were washed, and then treated with the indicated concentrations of devimistat as shown in FIG. 2G for 10 hours in CBS2. At the end of this treatment, ATP levels were measured directly (FIG. 2G, left two line graphs) or media was replaced with serum-free RPMI to allow recovery of viable cells for 20 hours, followed by measurement of ATP levels to assess cell survival (FIG. 2G, right two line graphs) (see Methods and Example 1A, above). Representative statistical significance (p values) for differences between non-pre-fed (CBS2) and pre-fed in each of the plots are as follows:
PANC1: Upper left line graph: 5 mM glucose (<0.0001), oleate (<0.0001), combination (<0.0001).
   Upper right line graph: 5 mM glucose (ns), oleate (<0.0001), combination (<0.0001), oleate/oleate+glucose comparison (0.010).
AsPC1: Lower left line graph: 5 mM glucose (0.026), oleate (0.012), combination (<0.0001).
   Lower right line graph: 5 mM glucose (ns), oleate (0.0007), combination (<0.0001), oleate/oleate+glucose comparison (<0.0001).

The bar graphs in FIG. 2G present alternative plots of selected data from the line graphs in FIG. 2G. Representative statistical confidence (p values) are as follows:
PANC1: Upper left bar graph: 5 mM glucose (<0.0001), oleate (<0.0001), combination (<0.0001).
   Upper right bar graph: 5 mM glucose (ns), oleate (<0.0001), combination (<0.0001).
AsPC1: Lower left bar graph: 5 mM glucose (0.026), oleate (0.012), combination (<0.0001).
   Lower right bar graph: 5 mM glucose (ns), oleate (0.0007), combination (<0.0001).

Comparison of the acute metabolic and cell death effects in this experiment indicate that even relatively modest levels of residual ATP are consistent with survival at these drug doses and exposure times.

### Example 3—Evidence for Roles of Endogenous Nutrient Stores in Determining the Sensitivity/Resistance of Tumor Cells to Devimistat

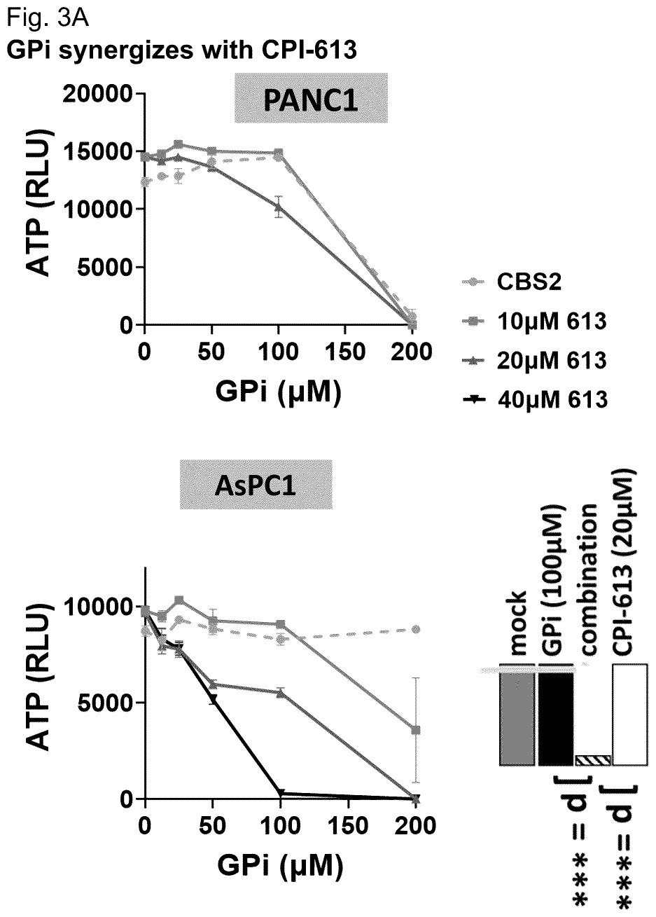

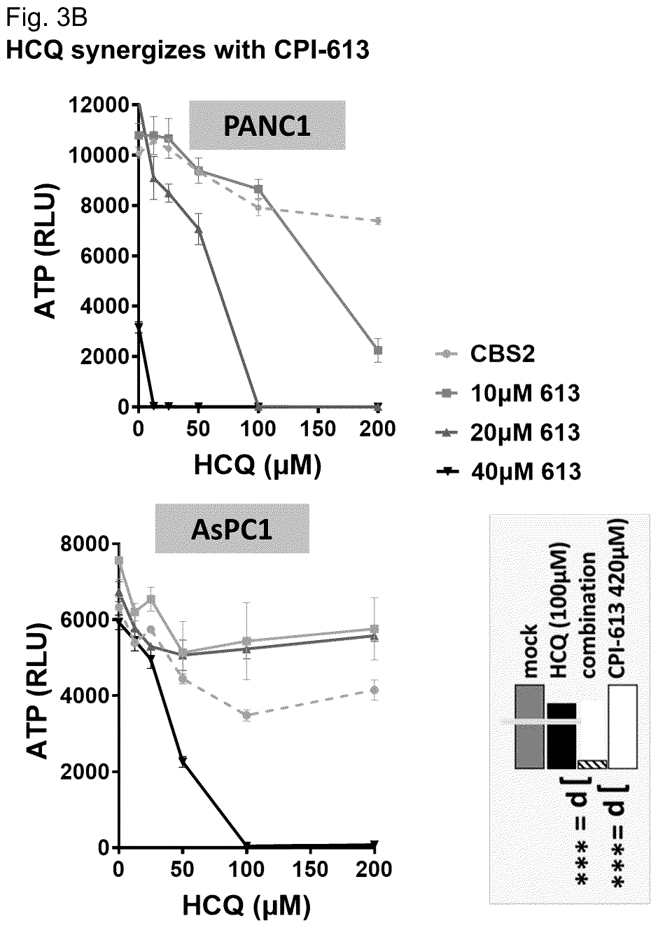

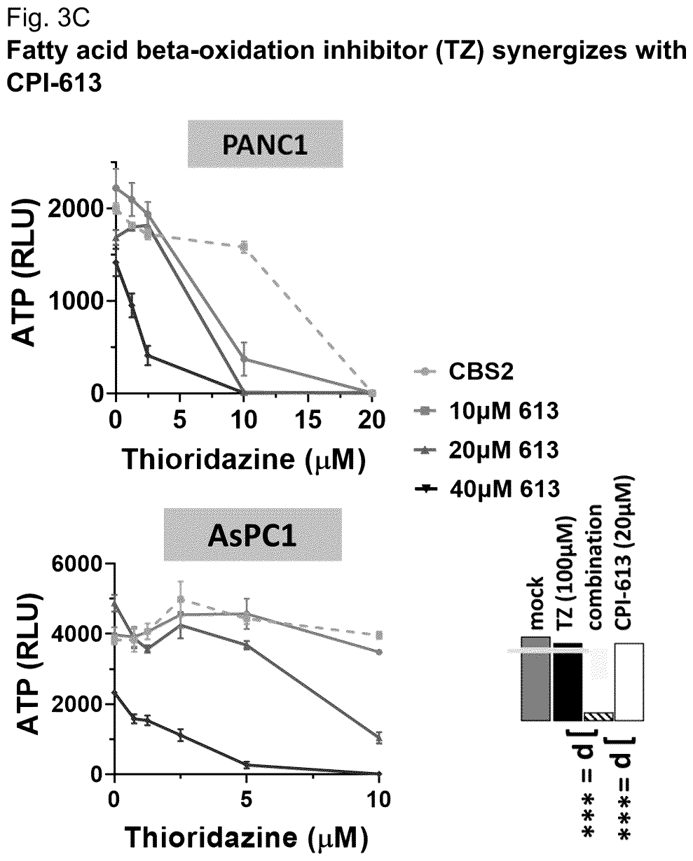

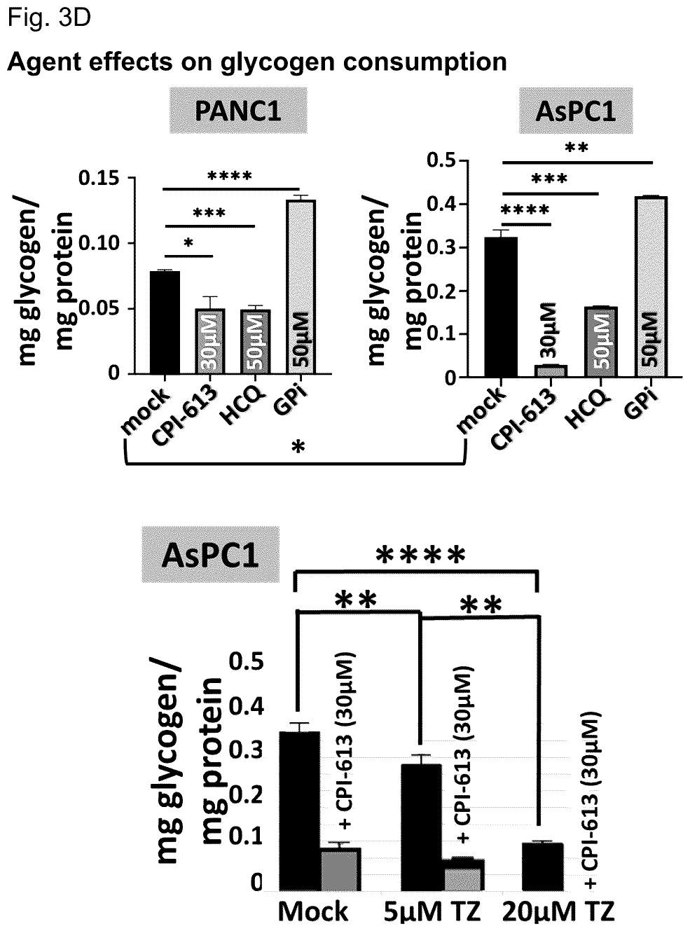

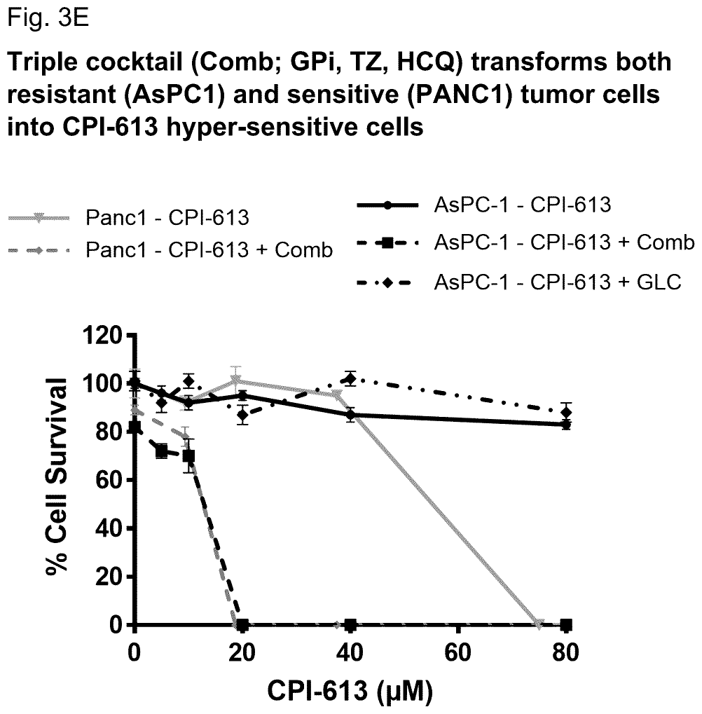

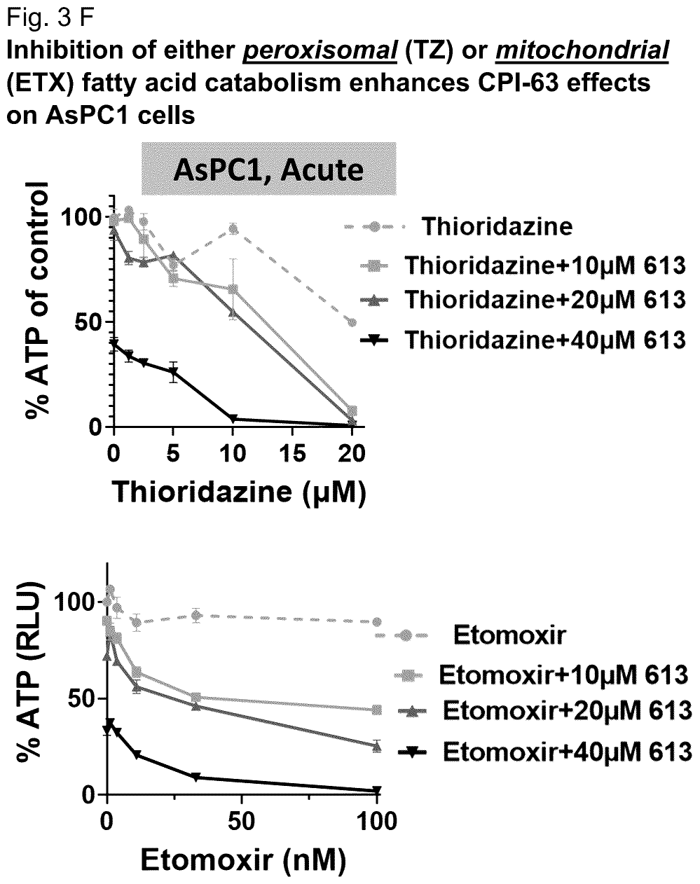

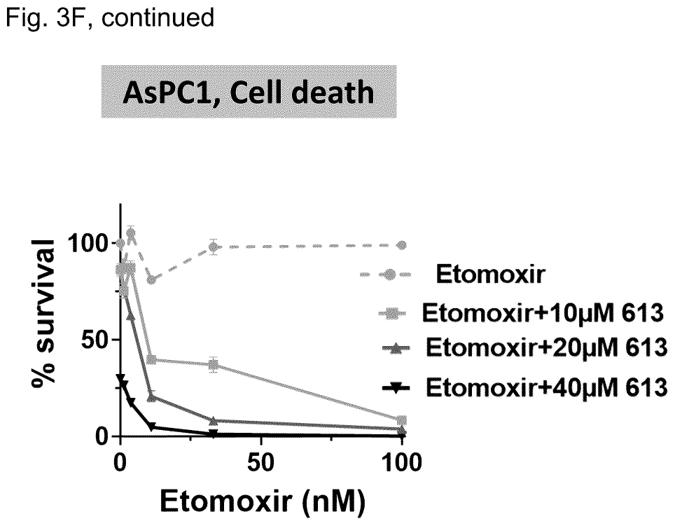

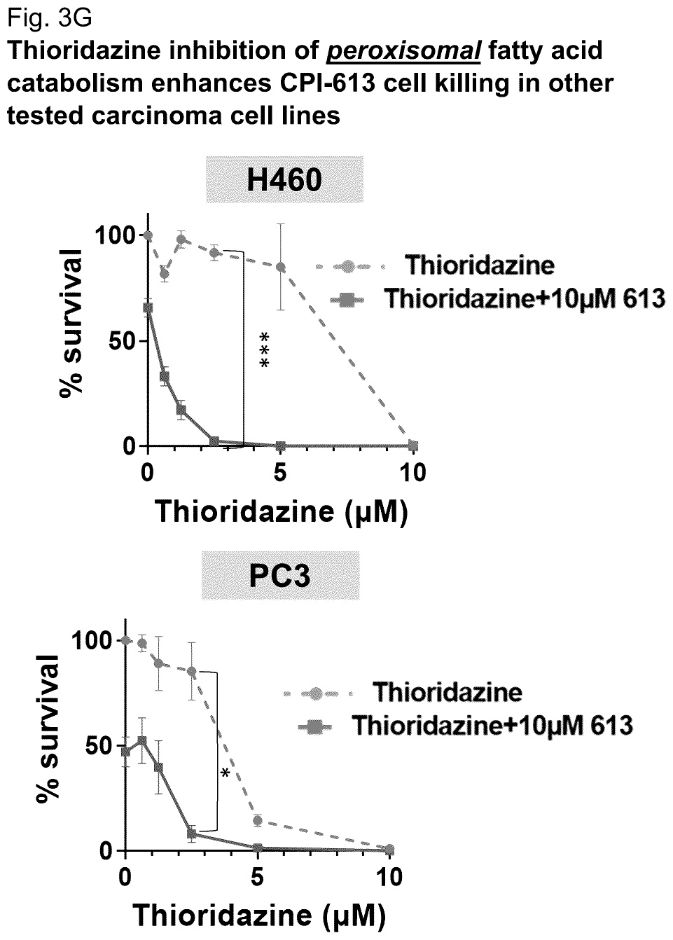

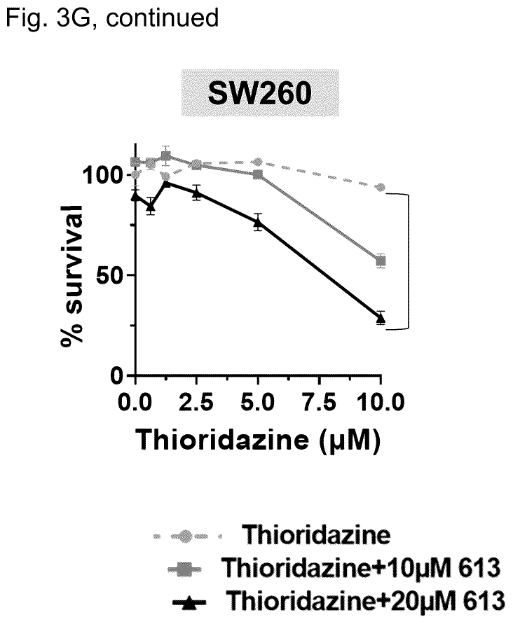

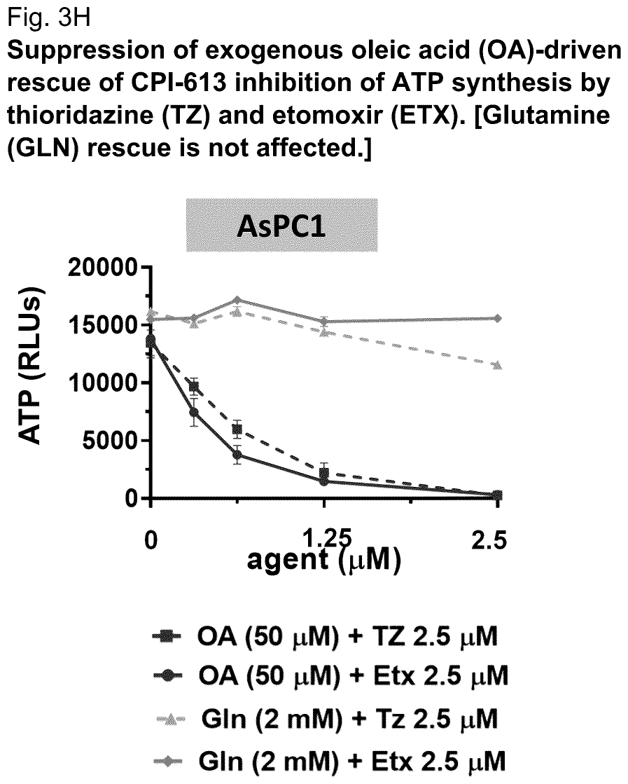

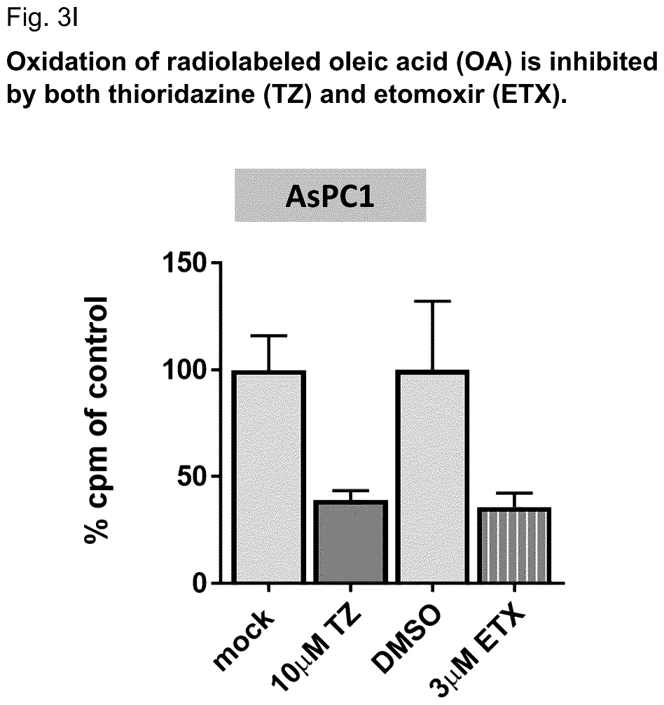

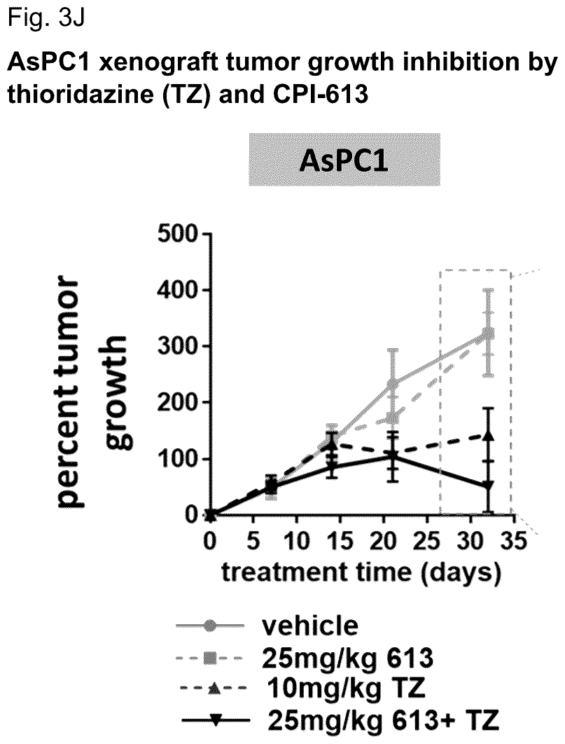

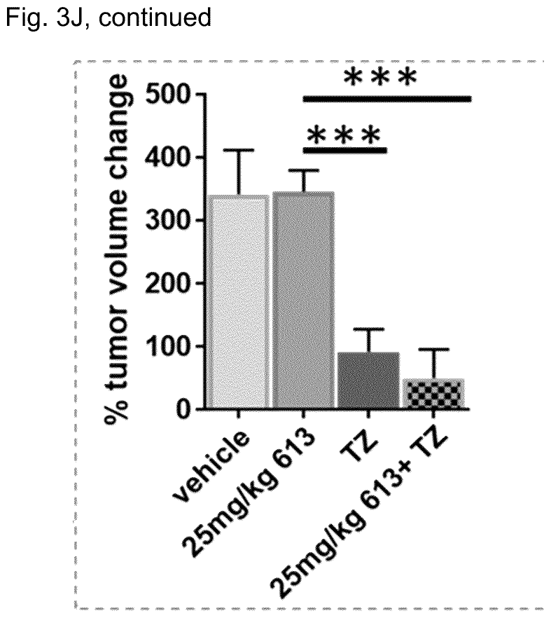

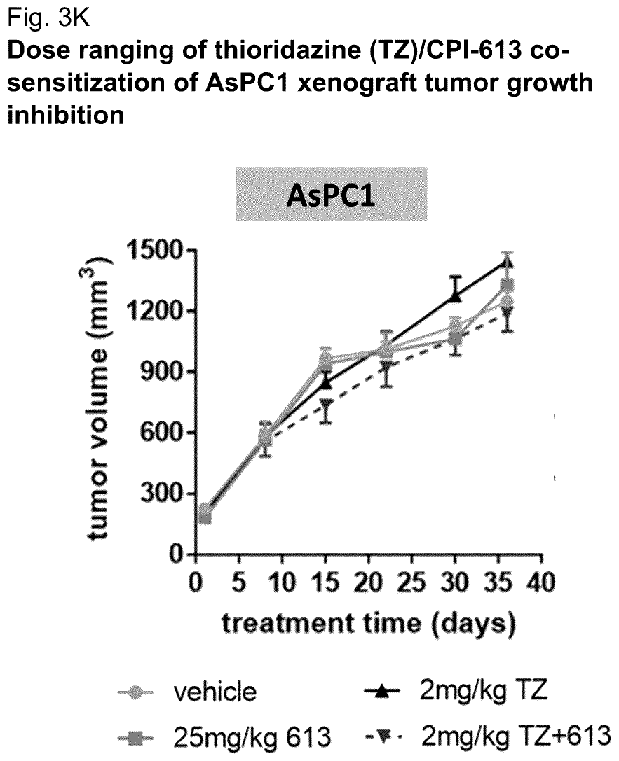

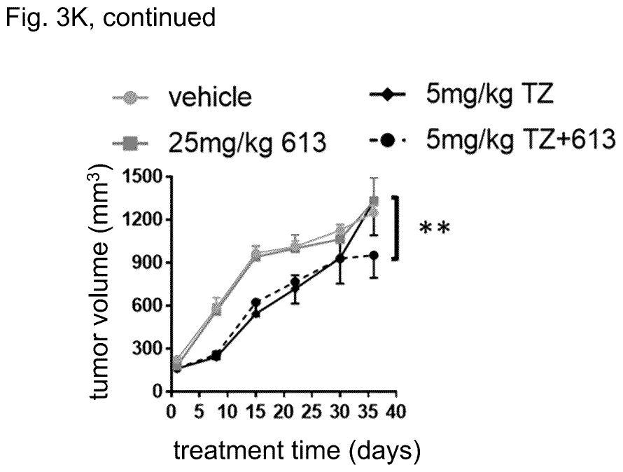

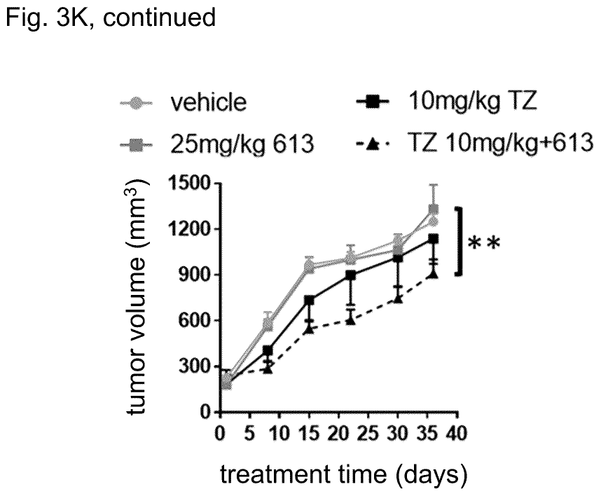

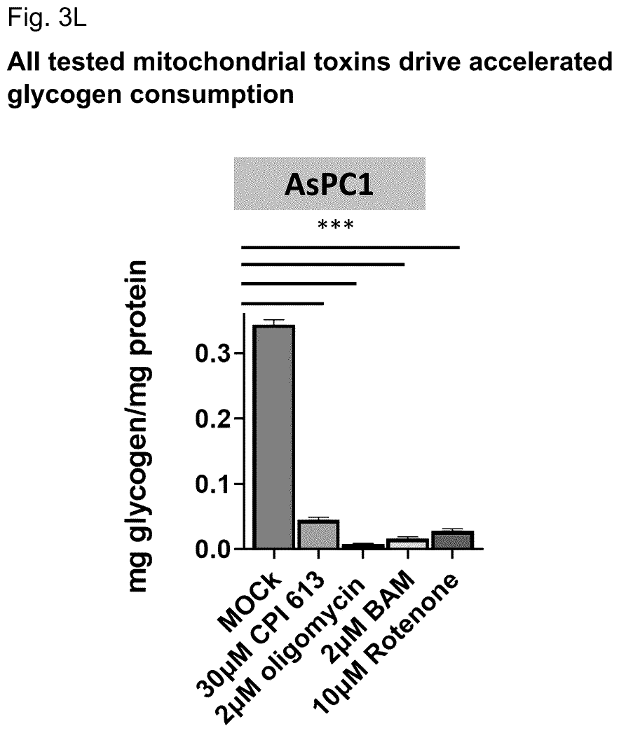

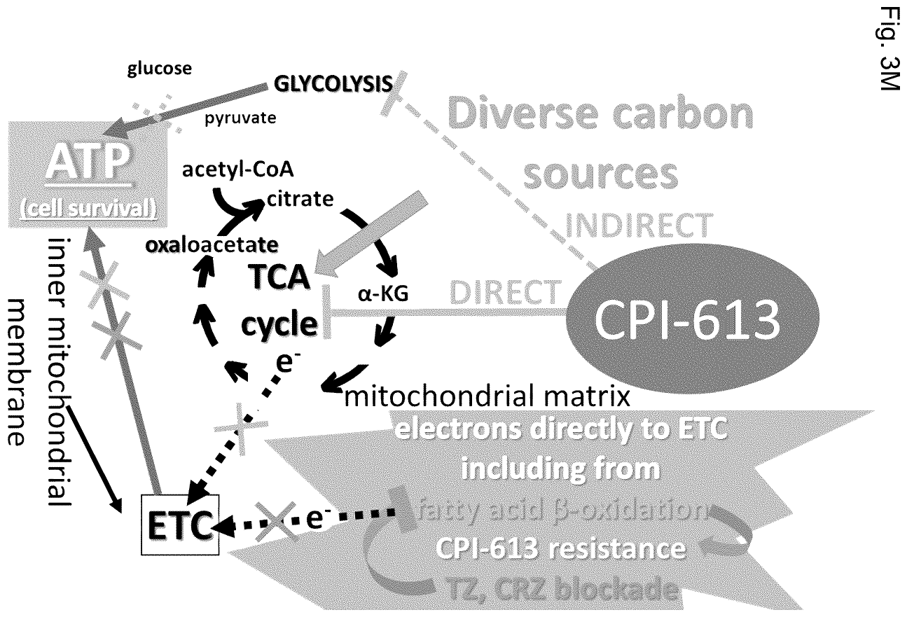

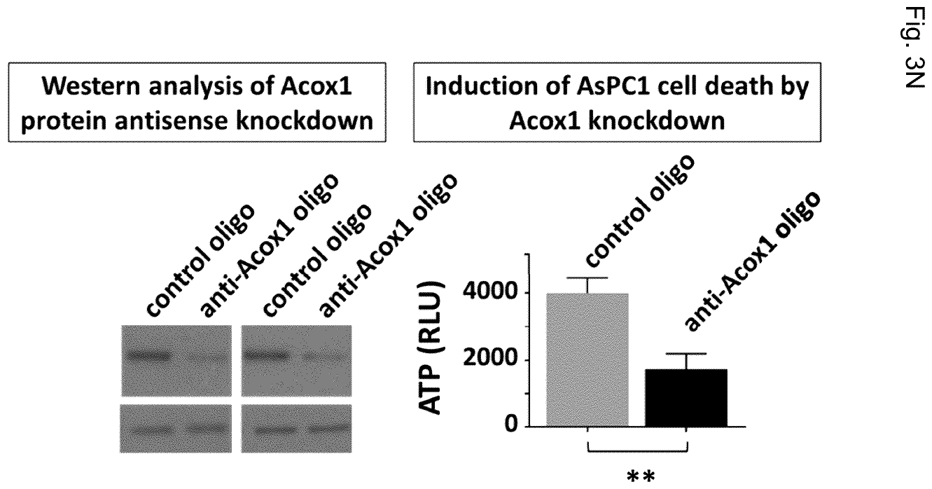

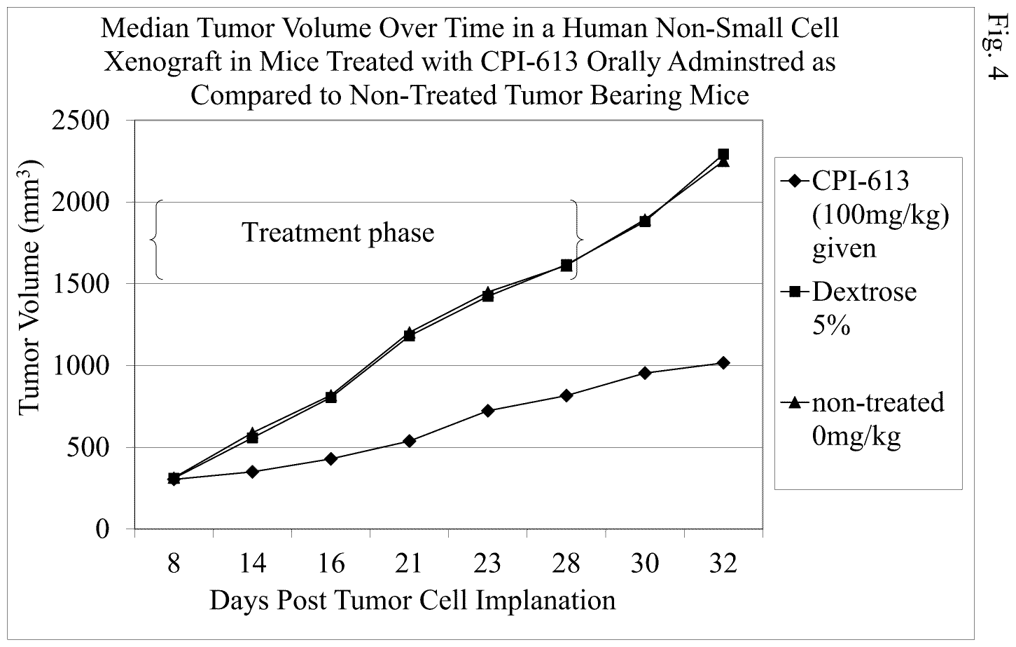

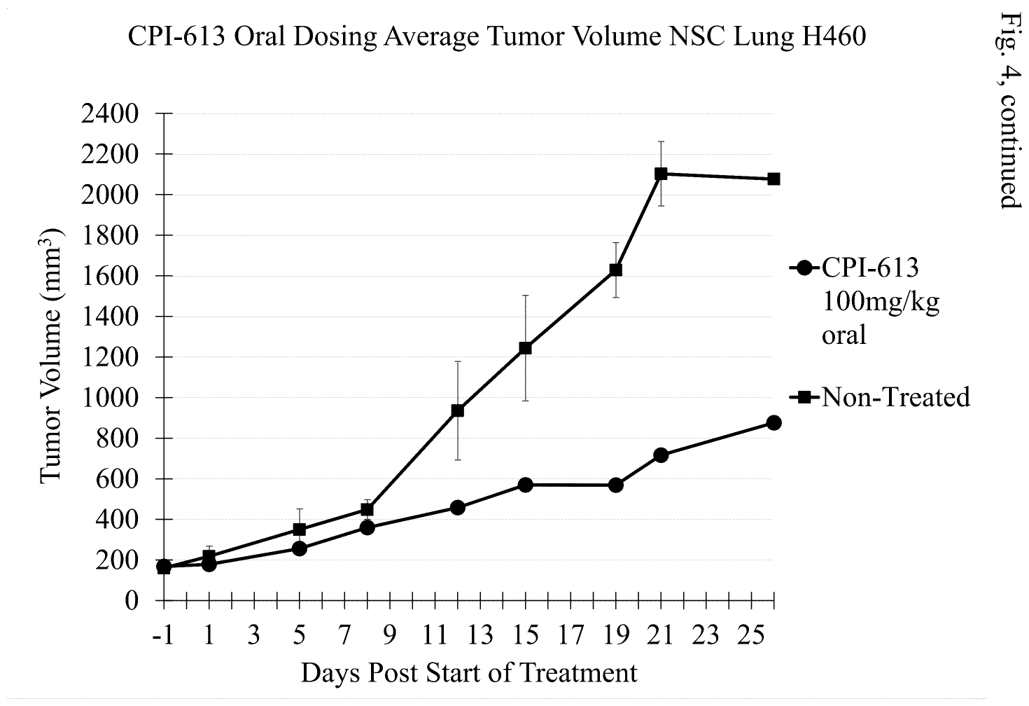

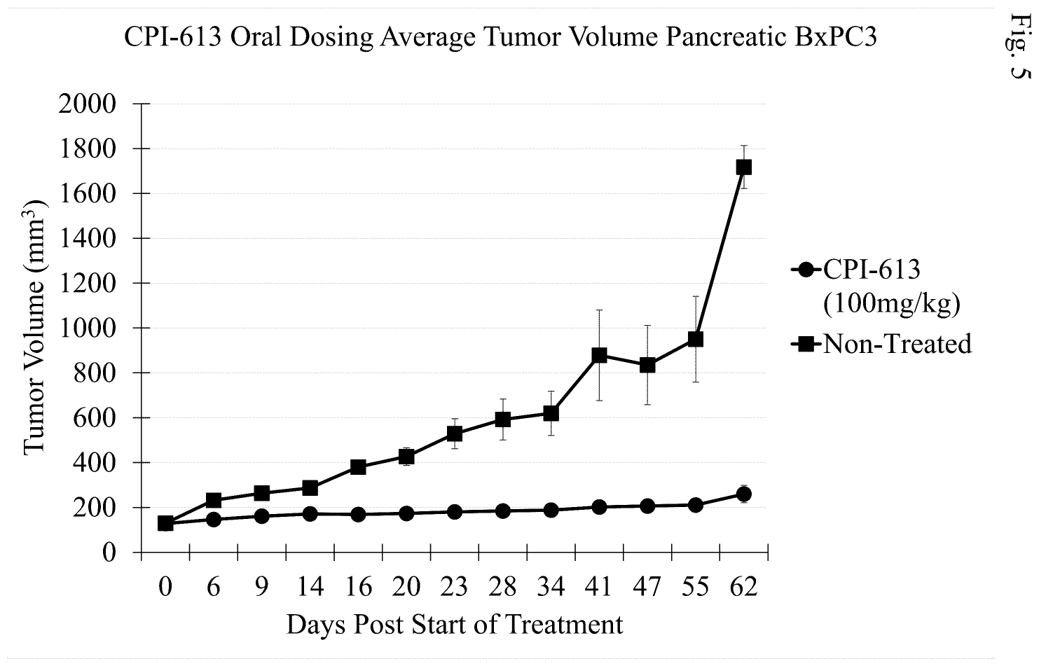

A. Effects of glycogen phosphorylase inhibitor (CP-91149; GPi) on devimistat sensitivity of PANC1 and AsPC1 cells. Cells were treated with a range of doses of GPi (horizontal axis) in the presence of several concentrations of devimistat as indicated in FIG. 3A for 21 hours, followed by 19 hours of recovery of viable cells in serum-free RPMI. ATP levels were measured (vertical axis) and results are presented in FIG. 3A. With reference to FIG. 3A, statistical confidence (p values) for grey boxed points is as follows: AsPC1 (40 μM devimistat+/−100 μM GPi=0.001). Synergy observed in AsPC-1 cells at the grey boxed points is presented in a bar graph on the right of FIG. 3A. The dotted horizontal line on the bar graph indicates the expected value for the combination of CP-91149 with devimistat in the absence of synergy (independent effects of devimistat and CP-91149 combination).

B. Effects of autophagy inhibitor hydroxychloroquine (HCQ) on devimistat sensitivity of PANC1 and AsPC1 cells. Cells were treated with a range of doses of HCQ (horizontal axis) in the presence of several concentrations of devimistat as indicated in FIG. 3B for 16 hours, followed by 20 hours of recovery of viable cells in serum-free RPMI. ATP levels were measured (vertical axis) and results are presented in FIG. 3B. With reference to FIG. 3B, statistical confidence (p values) for grey boxed points is as follows:
PANC1: 20 μM devimistat+/−100 M HCQ=0.0008.
AsPC1: 40 μM devimistat+/−100 M HCQ=0.001.

Synergy observed in PANC-1 and AsPC-1 cells at the grey boxed points are presented in bar graphs on the right of FIG. 3B. The dotted horizontal line on each bar graph indicates the expected value for the combination of HCQ with devimistat in the absence of synergy (independent effects of devimistat and HCQ combination).

C. Effects of peroxisomal fatty acid oxidation inhibitor thioridazine (TZ) on devimistat sensitivity of PANC1 and AsPC1 cells. Cells were treated with a range of doses of TZ (horizontal axis) in the presence of several concentrations of devimistat as indicated in FIG. 3C for 16 hours, followed by 20 hours of recovery of viable cells in serum-free RPMI. ATP levels were measured (vertical axis). ATP levels were measured (vertical axis) and results are presented in FIG. 3C.

Synergy observed in PANC-1 and AsPC-1 cells at the indicated concentrations of TZ and devimistat are presented in bar graphs on the bottom and right of FIG. 3C with statistical confidence (p values). The dotted line on each bar graph indicates the expected value for the combination of TZ with devimistat in the absence of synergy (independent effects of devimistat and TZ combination).

D. Effects of anti-metabolism agents on glycogen consumption in PANC1 and AsPC1 cells. PANC-1 and AsPC-1 cells were treated in the absence of exogenous nutrients (CBS2) for 3 hours with devimistat, HCQ, or CP-91149 (GPi) as indicated in FIG. 3D (plating sequence as in Example 1A). Glycogen content was measured in cell lysates (see Methods, above) and the results are presented in the left and middle graphs in FIG. 3D (note the different vertical scales for PANC1 and AsPC1 plots). These results show that CP-91149 reduces glycogen consumption in both PANC1 and AsPC1, indicating that glycogenolysis is a significantly active pathway for glycogen mobilization in both cell lines.

The results in the right bar graph in FIG. 3D indicate that TZ inhibition of fatty acid beta-oxidation accelerates glycogen consumption. Without wishing to be bound by theory, the TZ-accelerated glycogen consumption may represent a homeostatic regulatory response to inhibition of lipid-dependent metabolism.

* indicates p<0.05, ** indicates p<0.01, *** indicates p<0.001, **** indicates p<0.0001.

E. Effects of the combination of CP-91149 (GPi), HCQ, and TZ on the sensitivity of PANC1 and AsPC1 cells to devimistat-induced cell death. With reference to FIG. 3E, cells were treated for 16 hours in the absence of exogenous nutrients (CBS2) with the indicated concentrations of devimistat alone (solid lines) or in the presence of a cocktail of GPi (50 μM), HCQ (100 μM), and TZ (5 μM) (dashed lines), followed by 20 hours of recovery of viable cell in serum-free RPMI (see Methods and Example 1A, above). 5 mM glucose (supporting glycolytic ATP generation) protects AsPC1 from the devimistat/cocktail combination.

F. Inhibition of either peroxisomal (TZ) or mitochondrial (ETX; etomoxir) fatty acid catabolism enhances devimistat effects in AsPC1 cell lines. AsPC1 cells were treated with thioridazine (TZ) or etomoxir (ETX) alone or combined with devimistat (CPI-613) at various concentrations as indicated in FIG. 3F. Assays were carried out in exogenous nutrient-free CBS2 as described in Example 1A. Acute measurements were made after 4 hours of treatment. Cell death measurements were made after 19 hours of treatment followed by 24 hours of recovery in serum free RPMI. Results are presented in FIG. 3F. Relevant statistical significance levels (p values) are as follows:
ACUTE: 10 μM devimistat+33.3 nM ETX vs. 10 μM devimistat alone (<0.004)
ACUTE: 10 μM devimistat+33.3 nM ETX vs. 33 nM ETX alone (<0.009)
ACUTE: 40 μM devimistat+10 μM TZ vs 40 μM devimistat alone (<0.01)
ACUTE: 40 μM devimistat+10 μM TZ vs 10 μM TZ alone (<0.00007)
CELL DEATH: 20 μM devimistat+33.3 nM ETX vs. 20 μM devimistat alone (<3×10⁵)
CELL DEATH: 20 μM devimistat+33.3 nM ETX vs. 33 nM ETX alone (<0.003).

G. Thioridazine (TZ) inhibition of peroxisomal fatty acid catabolism enhances devimistat cell killing in other cell lines. H460 (lung cancer), PC3 (prostate cancer), and SW620 (colon cancer) cell lines were treated with thioridazine (TZ) alone or in combination with devimistat (10 μM devimistat; CPI-613) as in Example 3F except with 21 hours of treatment and 22 hours of recovery. Results are presented in FIG. 3G.

H. Suppression of exogenous oleic acid-driven rescue from devimistat inhibition of ATP synthesis by thioridazine (TZ) and etomoxir (ETX). Oleic acid (OA; 50 μM) or glutamine (GLN; 2 mM) were added to CBS2 to rescue AsPC1 cells from the suppression of ATP synthesis by 50 μM GPi (CP-91149) and 60 μM devimistat as indicated in FIG. 3H (6 hours treatment; black chevron indicates absence of exogenous carbon). Rescue by OA, but not by GLN, is strongly inhibited by either TZ or ETX. The exogenous carbon-free data (black line) show TZ titration result. Statistical significance (p values) for difference between 0 μM and 1.25 μM for each agent are 0.001317 for TZ and 0.0017982 for ETX.

I. Effects of thioridazine (TZ) and etomoxir (ETX) on CO₂ release from oleate. AsPC1 cells were treated in CBS2 with 50 μM GPi and 40 μM oleic acid (OA) for 1.5 hour and pulsed with 0.1 μCi of ¹⁴C-labeled OA during the final 0.5 hours of this treatment in the presence or absence of thioridazine (TZ) or etomoxir (ETX) as indicated in FIG. 3I. Released radiolabeled CO₂ was captured and measured (see Methods, above), and results are presented in FIG. 3I.

J. Treatment of AsPC1 xenografts with devimistat, thioridazine, or the combination thereof. Mice bearing AsPC1 xenograft tumors inoculated subcutaneously on the flank were intraperitoneally administered devimistat (CPI-613), thioridazine (TZ), or the combination thereof three days per week (MWF) at the doses indicated in FIGS. 3J and 3K (see Methods, above). Results are presented in FIGS. 3J and 3K. *** indicates p<0.001. * indicates p<0.05.

L. Mitochondrial inhibitors with diverse mechanisms of action induce glycogen store mobilization. AsPC1 cells were treated with devimistat (CPI-613), oligomycin (mitochondrial ATP synthase inhibitor), BAM (N⁵,N⁶-bis(2-Fluorophenyl)-[1,2,5]oxadiazolo[3,4-b]pyrazine-5,6-diamine; a mitochondrial membrane proton gradient uncoupler), or rotenone (ETC Complex I inhibitor) at the concentrations indicated in FIG. 3L in the absence of exogenous nutrients (CBS2) for 3 hours. Glycogen content was measured in cell lysates (see Methods and Example 1A, above), and the results are presented in FIG. 3L.

N. Anti-sense knockdown of Acox1 protein levels confirms that inhibition of Acox activity in responsible for TZ effects on devimistat sensitivity. Acox1 protein levels were knocked down with antisense morpholino oligos (METHODS). Control oligo was used at 2.5 μM and the anti-Acox1 oligos were a mixture of two independently targeting the Acox1 message (each at 1.25 μM). Cell survival measurement (ATP levels, FIG. 3N, right) were done as in Example 1A with 21 hrs in CBS2 followed by 24 hrs of recovery.

### Example 4—Oral Efficacy of Devimistat in Non-Small Cell Lung Cancer

Human H460 NSCLC cells were obtained from American Type Cell Culture (ATCC) (catalog no. HTB-177, Manassas, VA). These cells tested negative for viral contamination using the Mouse Antibody Production (MAP) test, performed by Charles River Labs Molecular Division, upon the receipt of the tumor cells from ATCC. The tumor cells were maintained at 37° C. in a humidified 5% CO₂ atmosphere in T225 tissue culture flasks containing 50 mL of Roswell Park Memorial Institute (RPMI)-1640 solution with 10% Fetal Bovine Serum (FBS) and 2 mM L-glutamine. Cells were split at a ratio of 1:10 every 2-3 days by trypsinization and resuspended in fresh medium in a new flask. Cells were harvested for experiments in the same way at 70-90% confluency.

CD1-Nu/Nu female mice, ˜4-6 weeks old were obtained from Charles River Laboratories. Mice were housed 5 to a cage in a micro-isolator room in the Department of Animal Laboratory Research of New York State University (SUNY) at Stony Brook. Light-dark cycles were 12 h each daily, with light from 7 a.m. to 7 p.m. Food (Purina Rodent Chow) and water (distilled sterile-filtered water, pH 7) were provided ad libitum. Protocols and procedures were according to the rules of and approved by the SUNY Institutional Animal Care and Use Committee (IACUC).

An acclimation period of 7 days was allowed between the arrival of the animal at the study site before tumor inoculation and experimentation. Mice were inoculated subcutaneously (SC) in the right flank with 2×10⁶ human H460 NSCLC or BxPC3 pancreatic cancer cells that were suspended in 0.1 mL of Dulbeco's Phosphate Buffered Salt (PBS) solution using a 1 cc syringe with a 27⅝ gauge needle. Tumor dimensions (length and width) were measured daily before, during and after treatment (using Vernier calipers) and the tumor volume was calculated using the prolate ellipsoid formula: (length×width²)/2. Treatment with test or control articles began 8 days post tumor cell implantation when the tumor was approximately 300 mm³.

Oral dosing of devimistat was at 100 mg/kg with 11 animals per group. 100 mg of devimistat was suspended in a small volume 0.01-0.05N NaOH in 5% dextrose and titrated to pH 7.0 with 4% Glacial Acetic Acid to 50 mg/mL. Prior to administration the suspension was diluted with 5% dextrose to 12.5 mg/mL so that the animals received 100 mg/kg with a dose volume of about 0.2 mL delivered by gastric gavage. Post tumor cell implantation, mice were treated on day 8, day 15, day 22, and day 29.

A similar study was conducted in CD-1 nude mice (n=9) inoculated with 2×10⁶ BxPC-3 cells. The study was initiated when tumors reached an average size of 150 mm³ (day 0) and devimistat was administered at an oral dose of 100 mg/week for 4 weeks. A comparator arm (n=9) was conducted with IP treatment at a weekly dose of 25 mg/kg.

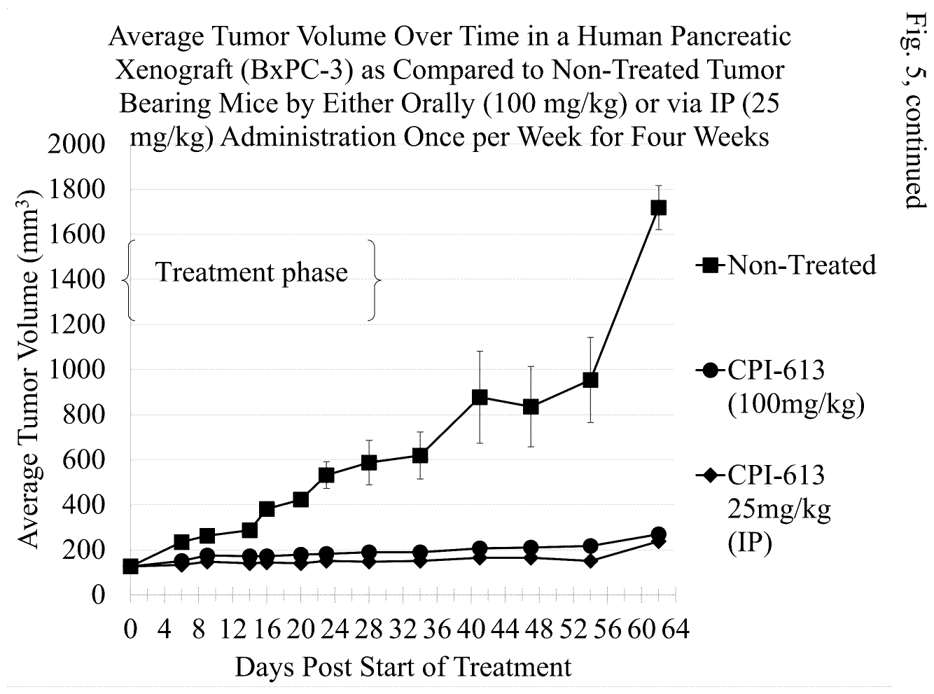

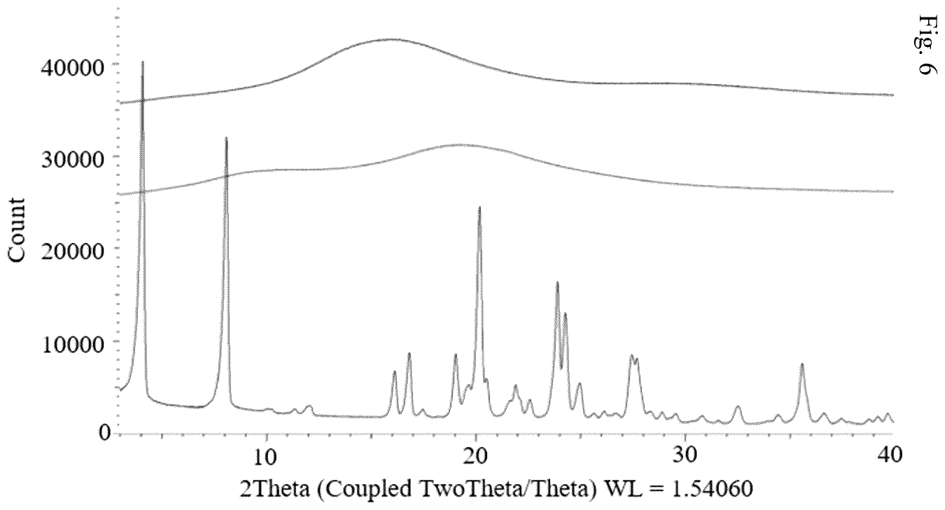

The results are presented in FIGS. 4 and 5. It is evident that the tumors in the mice treated with devimistat grew much more slowly than those in mice treated with 5% dextrose or untreated. The effect was especially pronounced in BxPC3 tumors. This example demonstrates that devimistat is effective to treat cancer when administered orally.

### Example 5—Spray Dried Dispersion Oral Formulation of Devimistat

Solid amorphous dispersion formulations of devimistat (API) were prepared by mixing the API 1:4 with one of the following polymers: Eudragit L100, poly(vinylpyrrolidone) viscosity grade K30 (PVP K30), hydroxypropyl methyl cellulose (HPMC), cellulose acetate phthalate (CAP), or hydroxypropyl methylcellulose acetate succinate (HPMCAS-M), and spray drying from methanol or acetone using a small-scale Bend Lab Dryer with 35 kg/hr drying gas flow rate capacity (BLD-35). Conditions, yields, and residual solvent levels of two representative spray dried dispersion (SDD) formulations (75 g each) are presented in the following table.

| | 20% API: | 20% API: |
|---|---|---|
| Formulation | Eudragit L100 | HPMCAS-M |
| Spray Solution | 5% solids in methanol | 5% solids in acetone |
| Outlet Temp | 45° C. | 35° C. |
| Solution | 35 | |
| Feed Rate | g/min | |
| Drying Gas | 475-500 | |
| Flow Rate | g/min | |
| Atomization | 120 | |
| Pressure | psi | |
| Nozzle | Schlick 2.0 pressure swirl atomizer | |
| Secondary Drying | 20 hr at 30° C. | |
| Dry Yield (%) | 94 | 96 |
| Residual Solvent (%) | 4.21 ± 0.02 | 1.01 ± 0.00 |
| (Wet SDD) | (MeOH) | (Acetone) |
| Residual Solvent (%) | <LOQ | <LOQ |
| (Tray-Dried Material) | | |
| API content by HPLC | 201 ± 1.1 mg/g | 198 ± 0.2 mg/g |

Scanning electron microscopy (SEM) was used to qualitatively determine particle morphology of the two SDD formulations, and to study if any degree of fusion or crystallinity was visually present. Particles show collapsed sphere morphology with no crystallization or fusion noted.

X-ray diffraction is typically sensitive to the presence of crystalline material with an LOD of about 1% of the sample mass. No crystallinity was detected by PXRD for either SDD formulation. Diffractograms in comparison to crystalline devimistat API can be found in FIG. 6, wherein the top diffractogram is the Eudragit L100 formulation, the middle diffractogram is the HPMCAS-M formulation, and the bottom diffractogram is crystalline devimistat.

### Example 6—Emulsion Oral Formulations of Devimistat

Monolaurin (131 mg) and devimistat (93 mg) were warmed to 50° C. in polysorbate-80 (2.5 mL) in a round bottomed flask equipped with a magnetic stir bar. After complete dissolution to a clear solution, water (7.5 mL) was added with vigorous stirring at 50° C. to provide an emulsion.

devimistat (312 mg) was combined with polysorbate 80 (6.25 g), soybean oil (1.25 g), and a lipid mix (100 mg) comprising cholesterol (14 g), cholesteryl acetate (14 g), cholesteryl benzoate (14 g), monolaurin (25.4 g), and monopalmitin (32.6 g), and the mixture heated to 50° C. until the solids dissolved (30 min). Dextrose (11.25 g) was dissolved in 236 mL of water, and the resulting aqueous dextrose solution was added to the oil solution above. The resulting two-phase mixture was stirred for 30 min at rt, then vacuum filtered through a 0.22 um filter.

### Example 7—Liquid Formulations of Devimistat

A devimistat solution was prepared by the steps of (a) providing a 50 mg/mL solution of devimistat in 1 M aqueous triethanolamine, and (b) diluting the 50 mg/mL solution with 5% aqueous dextrose to a concentration of 5 mg/mL. The resulting 5 mg/mL solution is identified as “7A” in Example 8 below.

A suspension vehicle was prepared by the steps of: (a) combining tris buffer (48 mg) and HPMCAS-HF (20 mg) in 14 mL of distilled water, (b) adjusting the pH to 7.4 with dilute sodium hydroxide to dissolve the HPMCAS-HF, (c) heating the resulting solution to approximately 90° C., (d) adding Methocel A4M Premium (100 mg) to the hot solution, (e) stirring the mixture vigorously to suspend the undissolved Methocel A4M, (f) cooling and stirring the mixture with an ice bath until the Methocel A4M dissolves (approximately 10 minutes), (g) diluting the solution with distilled/deionized water to bring the total volume to 20 mL, and (h) adjusting the pH to 7.4 with dilute acetic acid or dilute sodium hydroxide to provide the suspension vehicle.

Suspensions of the spray-dried formulations of Example 5 were prepared by adding 400 mg of the respective SDD formulation to a mortar, slowly adding 4 mL of the suspension vehicle (mixing thoroughly with a pestle after each small addition to uniformly disperse), and then transferring to a flask and stirring for one minute prior to administration. The resulting suspension of the Eudragit L100 SDD formulation (20 mg/mL devimistat) is identified as “7B” in Example 8 below. The resulting suspension of the HPMCAS-M SDD formulation (20 mg/mL devimistat) is identified as “7C” in Example 8 below.

In the same way, a 20 mg/mL suspension of devimistat was prepared by adding 80 mg devimistat to a mortar, slowly adding 4 mL of the suspension vehicle (mixing thoroughly with a pestle after each small addition to uniformly disperse), and then transferring to a flask and stirring for one minute prior to administration. The resulting suspension of devimistat is identified as “7D” in Example 8 below.

A solution of devimistat was prepared by dissolving SOLUTOL® (polyoxyl 15 hydroxystearate; KOLLIPHOR® HS 15) (3 grams) in distilled water (7 mL) to form a 30% solution, adding devimistat (50 mg) to 5 mL of the 30% solution, vortexing for 1 minute, and then sonicating for 45 minutes to provide a clear colorless solution (10 mg/mL; pH 7). The resulting solution is identified as “7E” in Example 8 below.

### Example 8—Oral Bioavailability of Devimistat

Six groups of 16 BALB/c nude mice (8 males and 8 females) per group were administered devimistat in six different ways: (1) 5 μL/g IV injection (tail vein) of the triethanolamine/dextrose aqueous solution of Example 7 (25 mg/kg; 5 mL/kg; Ex. 7A); (2) 5 μL/g IP injection of the triethanolamine/dextrose aqueous solution of Example 7 (25 mg/kg; 5 mL/kg; 7A); (3) 5 μL/g oral administration of the Eudragit L100 SDD suspension of Example 7 (100 mg/kg; 5 mL/kg; 7B); (4) 5 μL/g oral administration of the HPMCAS-M SDD suspension of Example 7 (100 mg/kg; 5 mL/kg; 7C); (5) 5 μL/g oral administration of the 20 mg/mL devimistat suspension of Example 7 (100 mg/kg; 5 mL/kg; 7D); or (6) 10 μL/g oral administration of the 10 mg/mL SOLUTOL solution of Example 7 (100 mg/kg; 10 mL/kg; 7E). In each experiment, about 80 μL of blood was collected from one subgroup of 4 male and 4 female mice at 0.083, 1, 4, and 24 hours after dosing, and from the other subgroup of 4 male and 4 female mice at 0.5, 2, and 8 hours. Plasma from the collected blood samples was analyzed by LC-MS/MS for the presence of devimistat.

| | | | | Bioavail- | AUC | | | T |
|---|---|---|---|---|---|---|---|---|
| | | Mice | Dose | ability | Last | Cmax | Tmax | ½ |
| Formulation | Route | (n) | (mg/kg) | (%) | (uM*hr) | (uM) | (hr) | (hr) |
| 7A (TEA/dextrose) | IV | 16 | 25 | — | 36 | 92 | 0.08 | 2.0 |
| 7A (TEA/dextrose) | IP | 16 | 25 | 83 | 29 | 103 | 0.08 | 3.9 |
| 7B (Eudragit SDD) | PO | 16 | 100 | 44 | 61 | 94 | 0.08 | 2.0 |
| 7C (HPMCAS-M | PO | 16 | 100 | 43 | 60 | 69 | 0.08 | 1.1 |
| SDD) | | | | | | | | |
| 7D (devimistat) | PO | 16 | 100 | 57 | 82 | 82 | 0.50 | 3.7 |
| 7E (Solutol) | PO | 16 | 100 | 127 | 175 | 229 | 0.08 | 4.4 |

This example demonstrates that devimistat is orally bioavailable.

### Example 9—Effect of Various Metabolic Agents and Inhibitors on Sensitivity to Devimistat

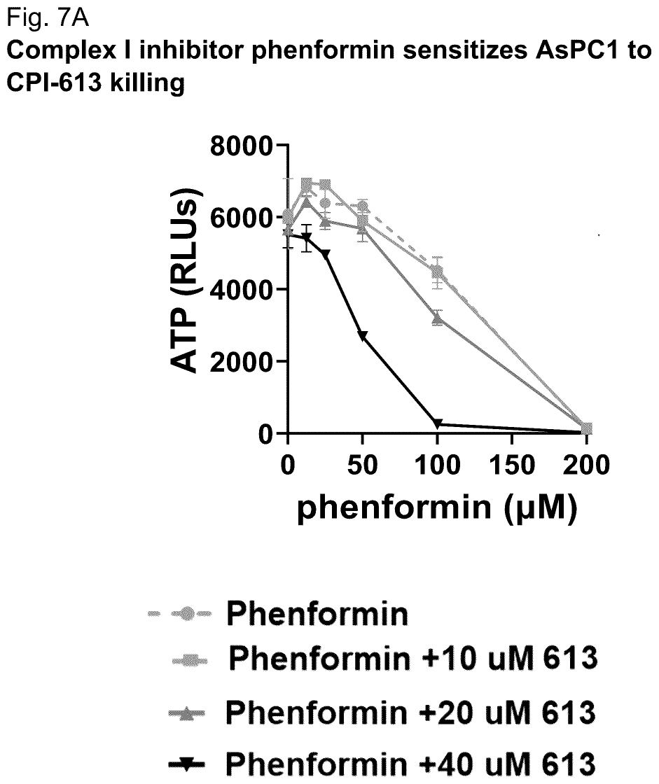

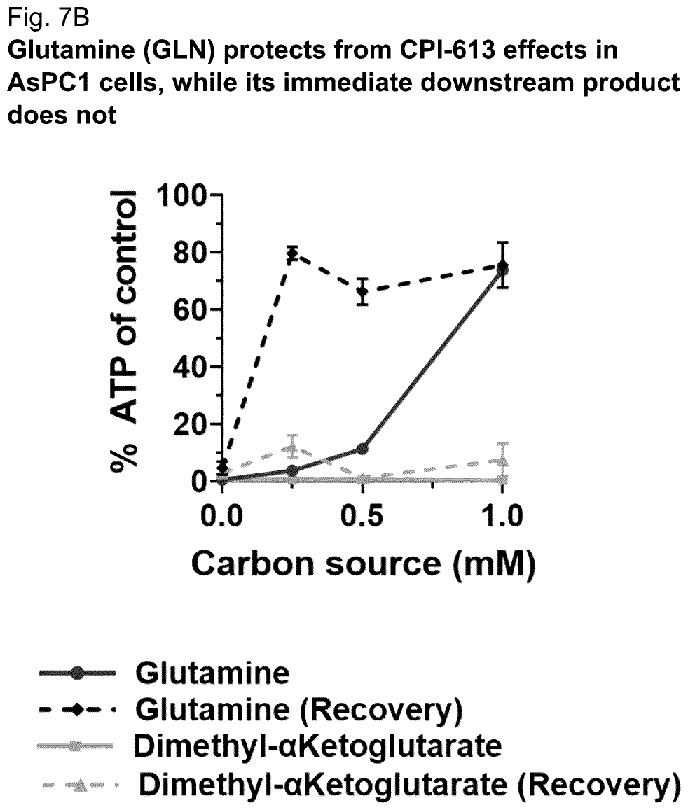

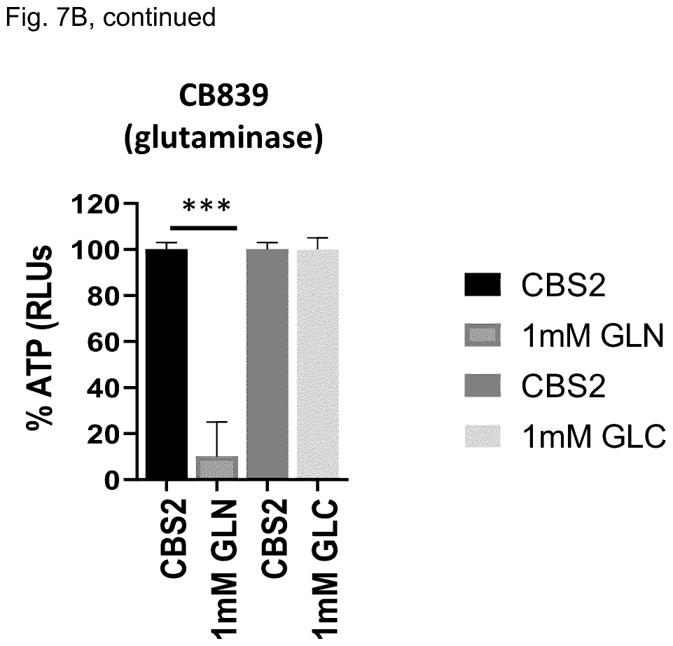

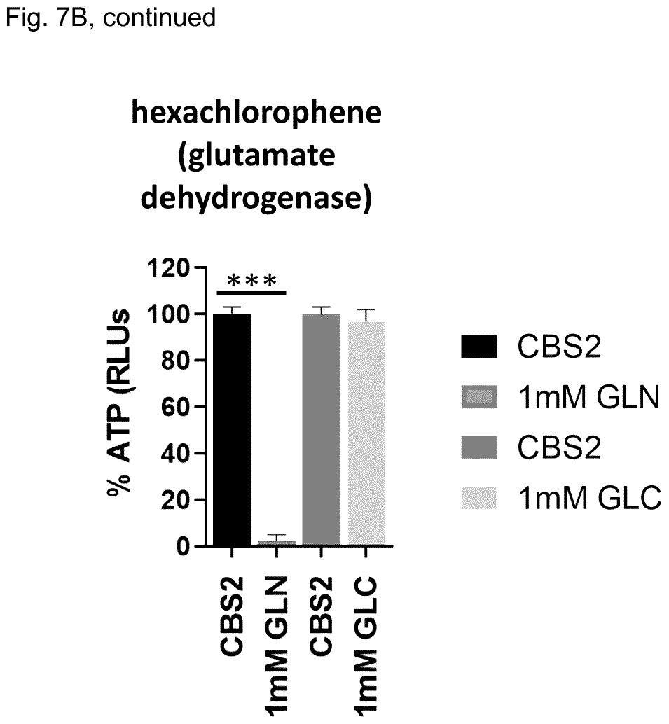

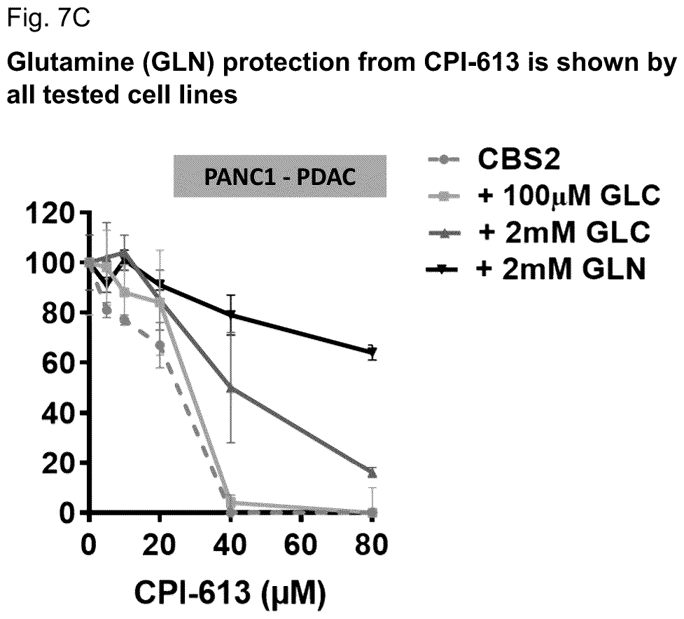

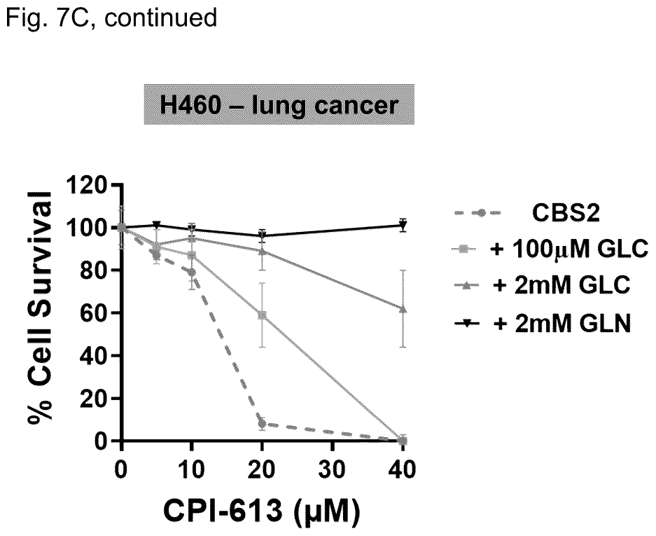

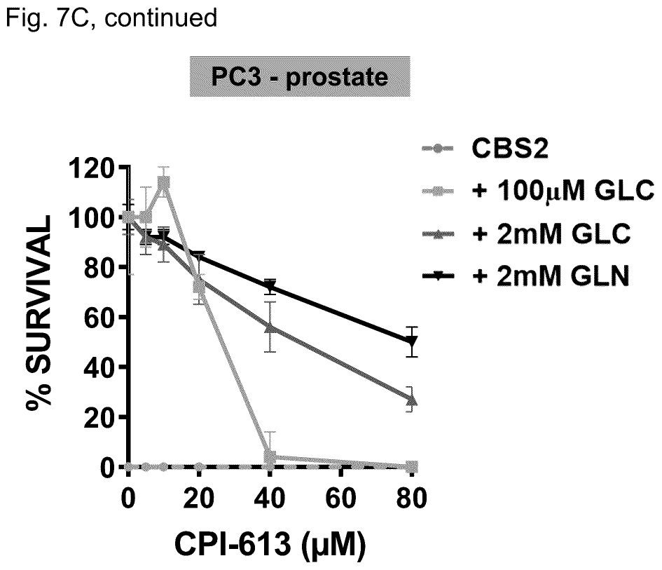

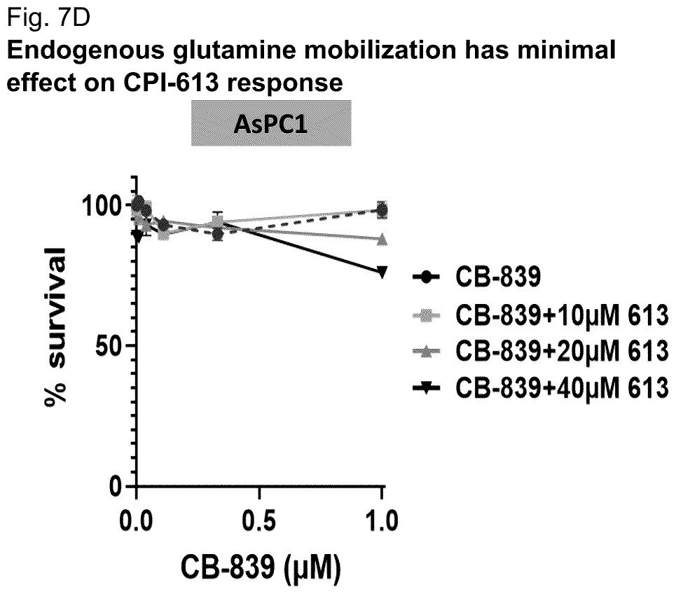

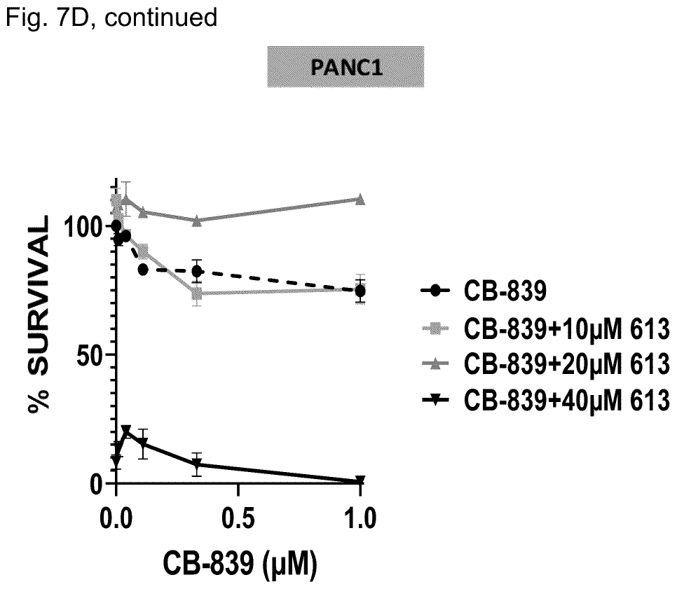

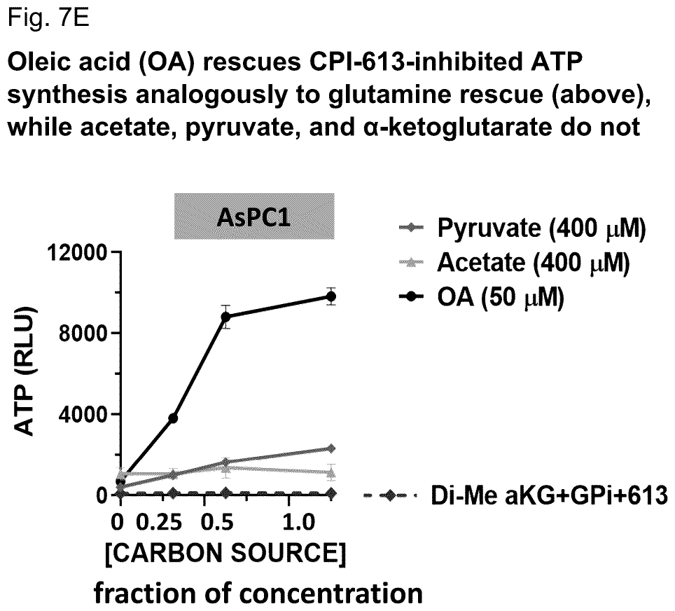

A. The complex I inhibitor phenformin sensitizes AsPC1 to devimistat. AsPC1 cells were treated for 20 hrs with devimistat (CPI-613) and phenformin as indicated in FIG. 7A, followed by 24 hrs of recovery before ATP measurement to assess cell death (see Methods and Example 1A, above). The results are presented in FIG. 7A, which shows the p values for differences between phenformin concentrations indicated alone and in presence of 40 μM devimistat.

B. Glutamine (GLN) protects AsPC1 cells from devimistat, but dimethyl-α-ketoglutarate, its immediate downstream product, does not. AsPC1 cells were treated with glutamine or dimethyl-α-ketoglutarate at the concentrations indicated in FIG. 7B (line graph) in the presence of 50 μM GPi (CP-91149) and 60 μM devimistat (CPI-613) to suppress ATP synthesis from endogenous sources. Acute measurements were taken after 4.5 hours of drug exposure and cell death commitment measurements after 15 hours of drug exposure followed by 22 hours of recovery in serum-free RPMI (see Example 1A and Methods, above), with results presented the line graph in FIG. 7B.

As shown in the FIG. 7B line graph, the cell-permeable dimethyl ester form of α-ketoglutarate (robustly delivering α-ketoglutarate through cytosolic esterase action; Baracco, E. E. et al., “alpha-Ketoglutarate inhibits autophagy,” Aging-Us, 2019, 11(11), 3418-3431) provided negligible protection from devimistat inhibition of ATP synthesis or induction of cell death, consistent with devimistat targeting of the TCA cycle. In contrast, glutamine (2 mM) strongly protects from both drug effects.

In additional experiments, AsPC1 cells were treated with CB-839 (0.2 μM) or hexachlorophene (HXP; 3 μM) and either glutamine (GLN; 2 mM) or glucose (GLUC; 5 mM) in the presence of 50 μM GPi (CP-91149) and 60 μM devimistat (CPI-613) in CBS2. Acute ATP measurements were taken after 4.5 hours of treatment, with results presented in the bar graphs in FIG. 7B.

As shown in the bar graphs in FIG. 7B, glutamine rescue from devimistat inhibition of ATP synthesis is dependent on glutaminase activity (as assessed by CB-839 blockade; 0.2 μM) and glutamate dehydrogenase activity (as assessed by hexachlorophene blockade; 3 μM), while glucose rescue (GLUC; 5 mM) is not.

C. Glutamine (GLN) protects PANC-1, PC-3, and H460 cells from devimistat. PANC-1 pancreatic cancer cells, PC-3 prostate cancer cells, and H460 lung cancer cells were treated with devimistat and optionally glutamine (GLN) at the concentrations indicated in FIG. 7C for 8 hours (PC-3) or 15 hours (PANC1, H460) under conditions as described in Example 1A, including post-treatment rescue to assess cell death commitment. Results are presented in FIG. 7C.

D. Endogenous glutamine mobilization has minimal effect on devimistat response. PANC-1 and AsPC1 cells were treated in CBS2 with CB-839 and/or devimistat (CPI-613) at the concentrations indicated in FIG. 7D for 15 hours, followed by 28 hours in serum-free RPMI to assess cell death. The results presented in FIG. 7D indicate that CB-839 inhibition of glutaminase/glutamine mobilization produces modest effects on devimistat sensitivity in the absence of exogenous nutrients, particularly in AsPC1.

E. Oleic acid rescues devimistat-inhibited ATP synthesis analogously to glutamine, but acetate, pyruvate, and α-ketoglutarate do not. AsPC1 cells were treated with devimistat (CPI-613; 60 μM), GPi (CP-91149; 60 μM) and either oleic acid (OA), acetate (Ac), pyruvate (Py), or dimethyl-α-ketoglutarate at the concentrations indicated in FIG. 7E for 6 hours. The results are presented in FIG. 7E. The statistical significance (p value) for the differences between 400 μM acetate and 50 μM oleic acid is <0.006 and for 400 μM pyruvate and 50 μM oleic acid is <0.0002. The results indicate that oleic acid rescues devimistat-induced ATP depletion in the presence of GPi in a concentration-dependent manner, but other tested carbon sources not providing electrons directly to the ETC do not (acetate, dimethyl-a-ketoglutarate, and pyruvate). (Note that pyruvate oxidation is blocked by devimistat effects on PDH.) Example 10—Tyrosine kinase inhibitors (RTKi's) mimic TZ inhibition of FA rescue and sensitize resistant AsPC1 tumor cells to devimistat in vitro and in vivo.

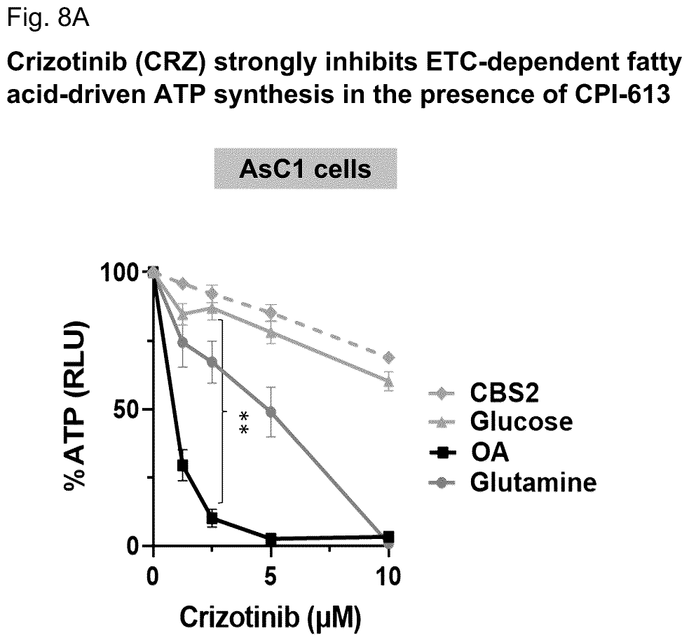

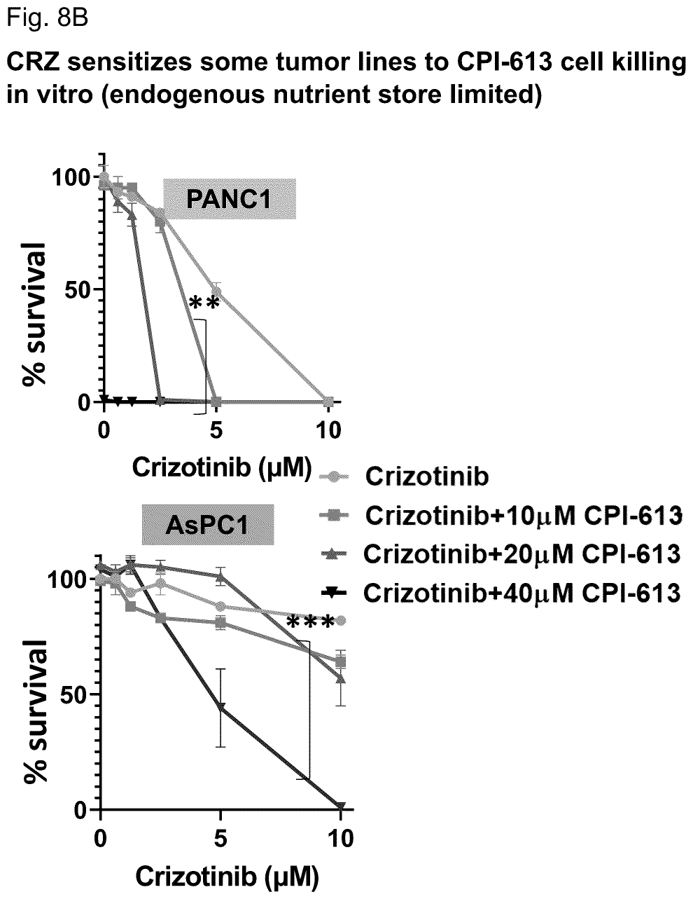

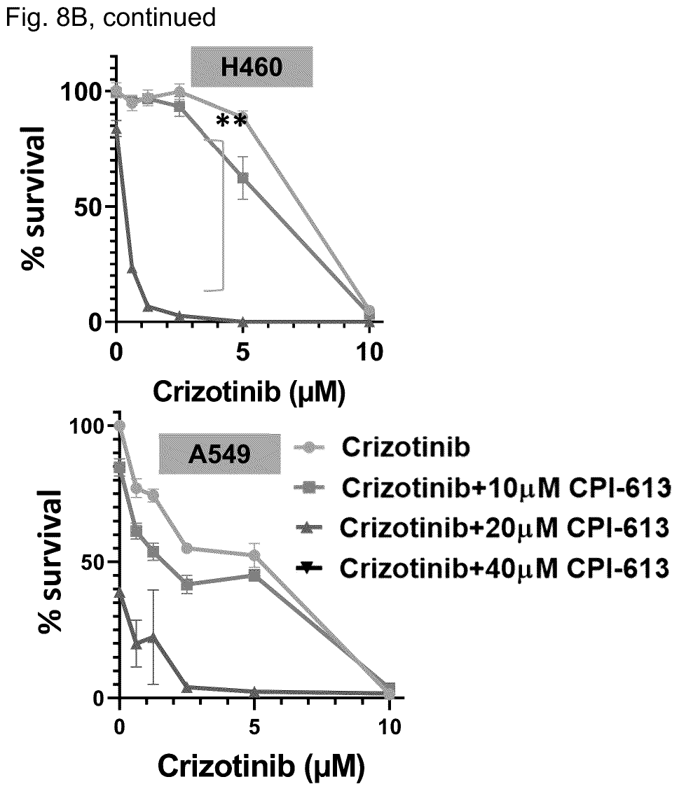

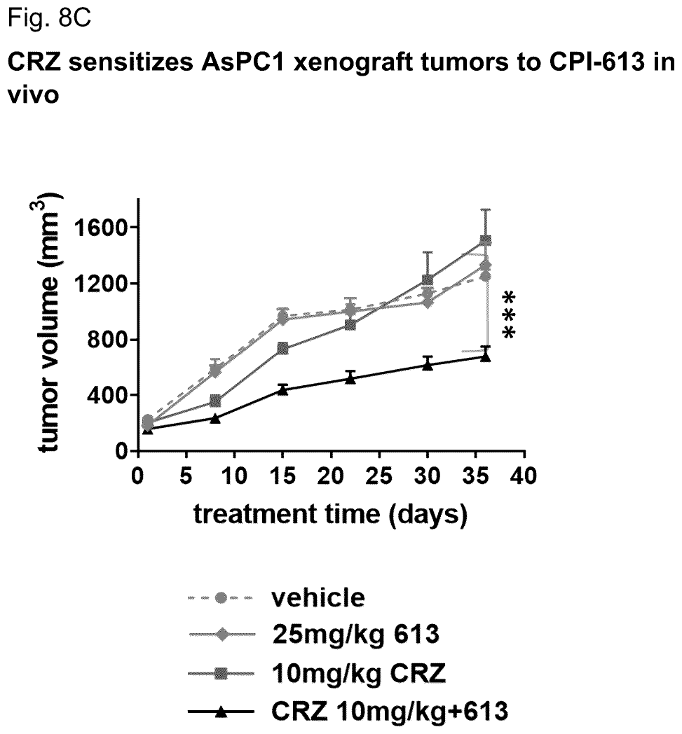

A. Crizotinib preferentially inhibits ETC-dependent fatty acid driven ATP synthesis. AsPC1 cells were treated for 4.5 hours in CBS2 with 50 μM GPi (CP-91149) and 60 μM devimistat as in Examples 3H and 9B, optionally in the presence of crizotinib (CRZ) and/or exogenous carbon sources as indicated in FIG. 8A (1 mM glucose, 1 mM glutamine, 50 μM oleic acid (OA)), or with CRZ without GPi or devimistat (CBS2 line). Raw acute ATP readings were normalized to respective untreated controls for each carbon source addition and the results are presented in FIG. 8A. Statistical significance for the difference between oleic acid-driven ATP generation in the absence of CRZ and in the presence of 2.5 μM CRZ is p<0.007.

B. Crizotinib sensitizes to devimistat killing in several cell lines. PANC1, H460, AsPC1, and A549 (lung cancer) cells were exposed to several concentrations of crizotinib (CRZ) and devimistat in the absence of exogenous carbon and GPi for 15 hours followed by 24 hours of recovery as indicated in FIG. 8B (see Example 1A and Methods, above). Assessment of cell death after treatment followed by recovery is presented in FIG. 8B (see Example 1 and Methods, above).

C. The in vitro binding affinities of crizotinib, PHA-665752, foretinib, PD-173955, and GSK1838705A for ALK, ROS1, and MET are presented in the following table, along with their effects on acute ATP synthesis (as in Example 10A) and devimistat-induced cell death (as in Example 10B).

| | | | | | | | OA rescue | Devimistat |
|---|---|---|---|---|---|---|---|---|
| RTKi | ALK | | ROS1 | | MET | | blockade | synergy |
| Crizotinib | 3.3 | nM | 4.1 | nM | 0.6-8 | nM | Yes | Yes |
| PHA-665752 | 3000 | mM | 2700 | nM | 0.3 | nM | Yes | Yes |
| Foretinib | 69 | nM | 14 | nM | 1.4 | nM | Yes | Yes |
| PD-173955 | >3000 | nM | >3000 | nM | 830 | nM | No | No |
| GSK1838705A | 0.6 | mM | 15 | nM | >3000 | nM | No | No |

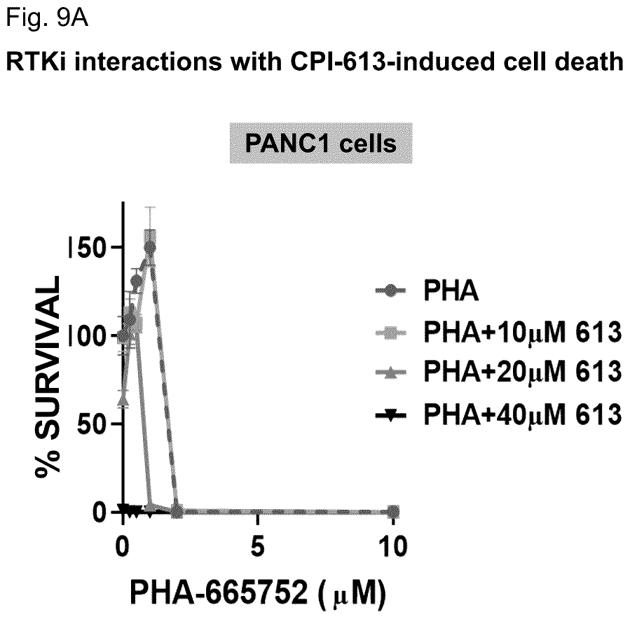

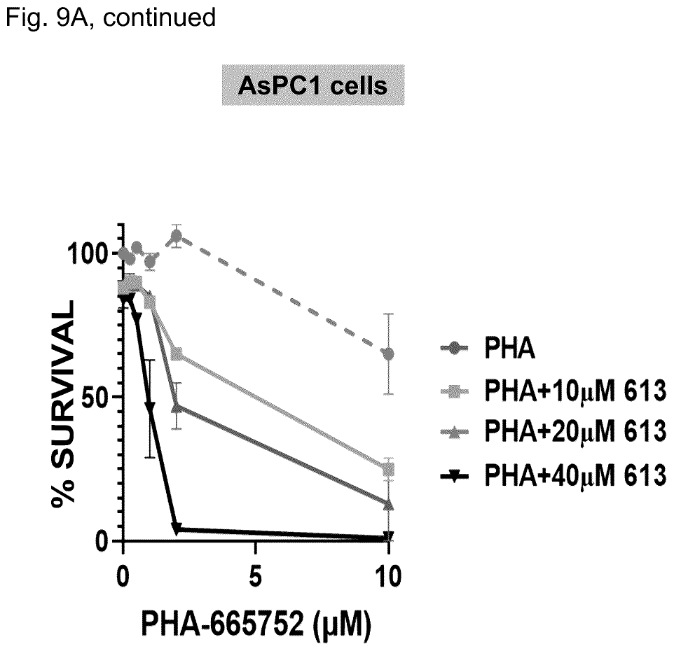

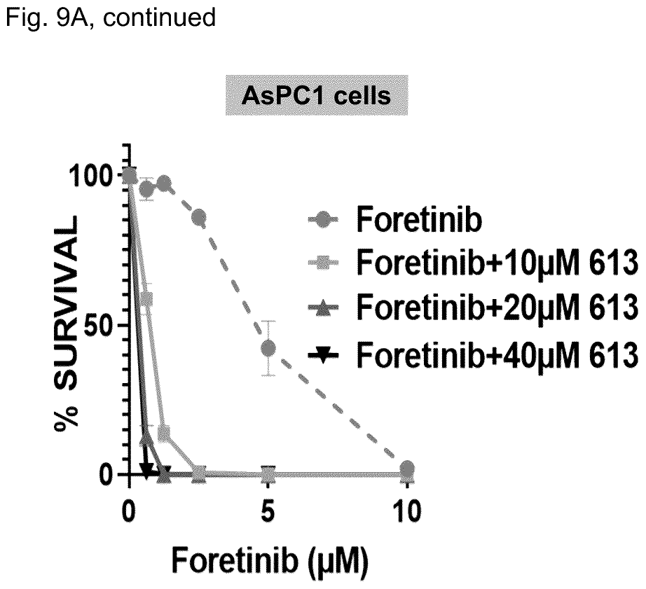

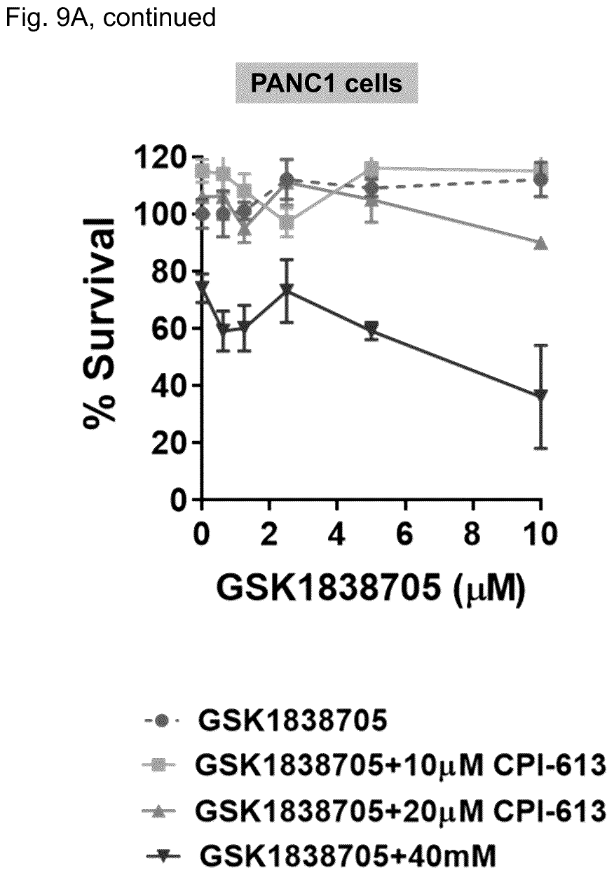

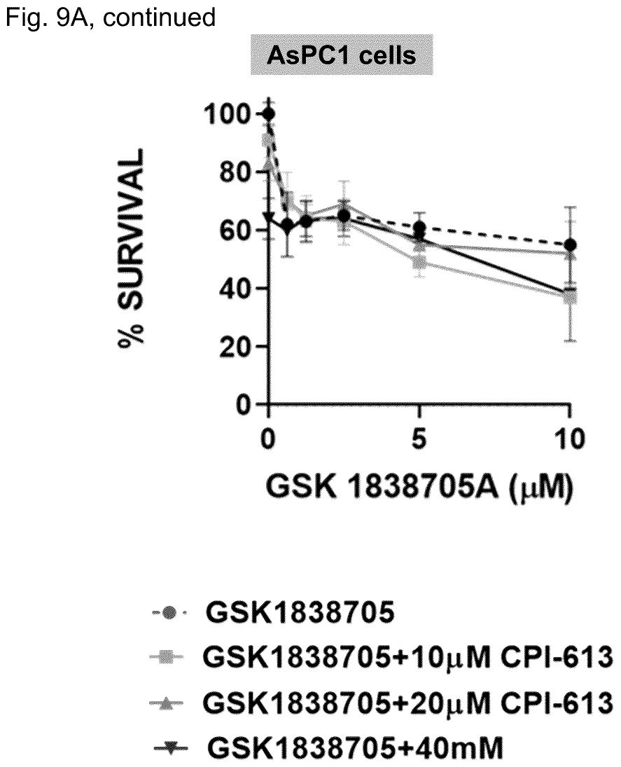

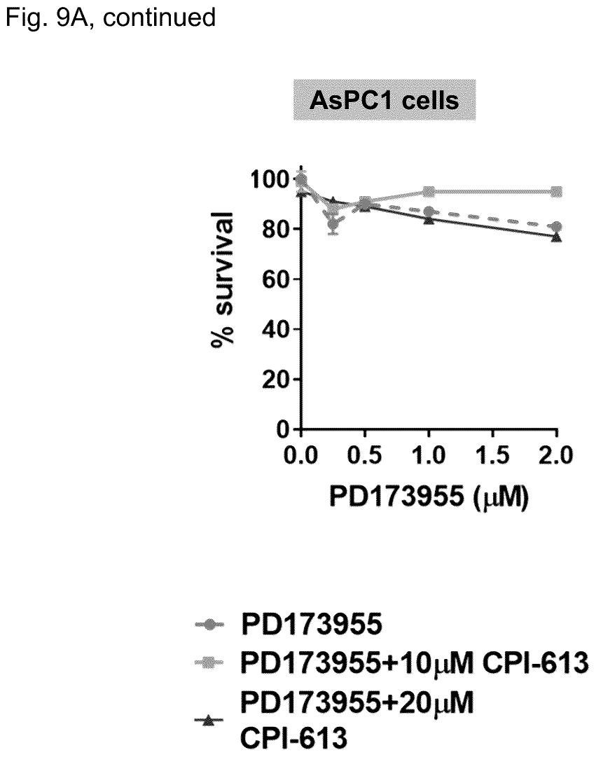

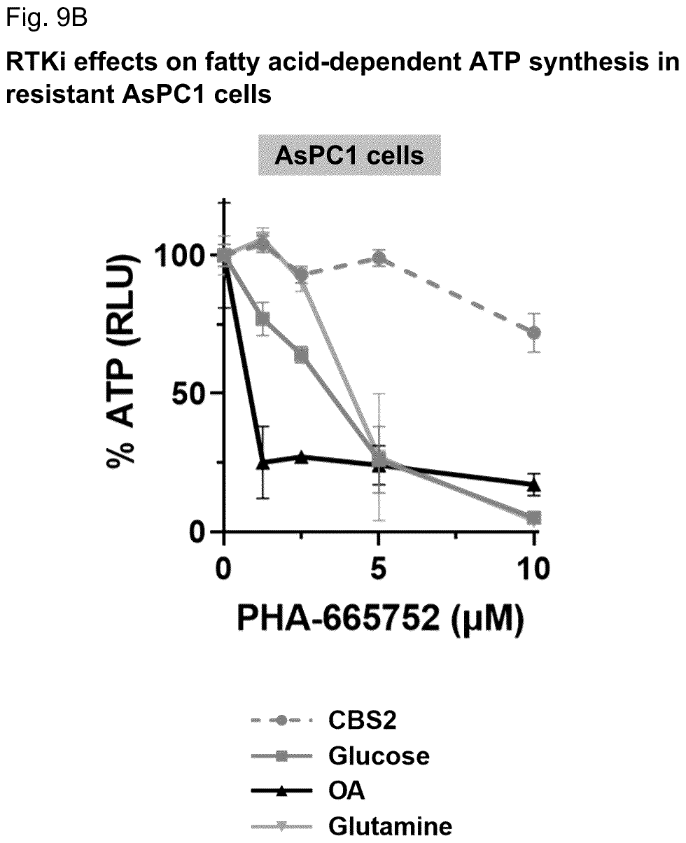

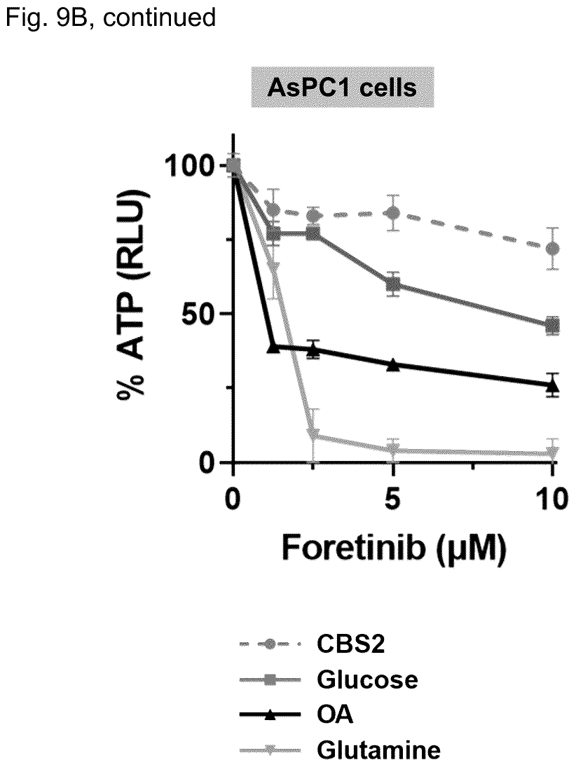

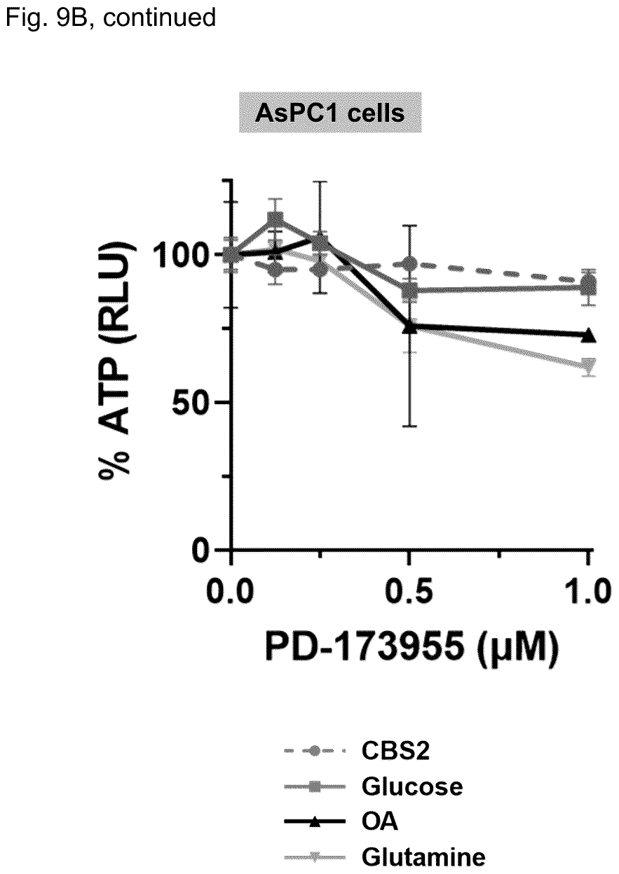

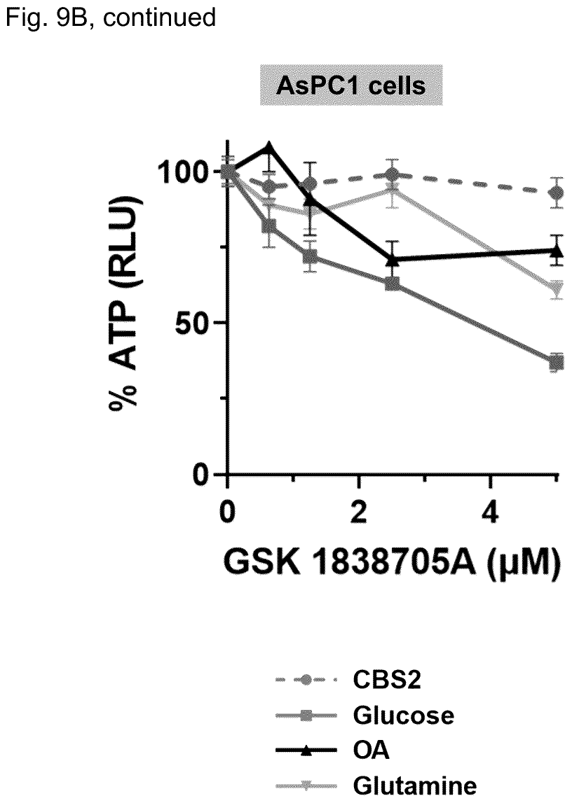

The in vitro binding affinities of each inhibitor for the ALK, ROS1, and MET kinases are indicated. FIGS. 9A, 9B contain some of the primary data summarized in the table. The tested RTKi high affinity MET inhibitors (CRZ, PHA-665752, foretinib) robustly sensitize to devimistat-induced cell death in the absence of exogenous carbon (FIG. 9A, left and FIG. 8B). In contrast, the two tested RTKi low affinity MET inhibitors (PD-173955, GSK1838705A) show more limited sensitization (FIG. 9A, right). Moreover, the effects observed in the low affinity inhibitors (FIG. 9A, right) do not mimic the response patterns seen with nutrient store mobilization inhibitors (FIG. 3A-C), indicating that these effects likely have unrelated mechanisms. These patterns of sensitization are supported by the acute data in FIG. 9B. Assessment of cell death (treatment followed by recovery as in Example 1A) induced by the indicated combinations of devimistat and RTKi's during 15 hours of treatment followed by 20024 hours recovery (reference FIG. 8B).

The tested RTKi high affinity MET inhibitors (CRZ, PHA-665752, foretinib) show robust inhibition of acute ATP synthesis driven by oleic acid (OA) (FIG. 9B, left and FIG. 8A). In contrast, the two the tested RTKi low affinity MET inhibitors (PD-173955, GSK1838705A) show little or no inhibition of OA-dependent ATP synthesis (FIG. 9B, right). (50 μM GPi; 60 μM CPI-613; 50 μM oleic acid; 1 mM glucose; 1 mM glutamine; treatment time 3 hours).

Each of the strong METi's also has a distinct pattern of effects on ATP synthesis driven by glucose and glutamine (*). These distinctions presumably reflect other metabolic targets idiosyncratic to each agent.

D. Assessment of CRZ and/or devimistat effects on AsPC1 xenograft tumor growth in vivo. Tumors were inoculated subcutaneously on the flank and drug combinations indicated in FIG. 8C were administered intraperitoneally (devimistat) or by oral gavage (CRZ) three days per week (MWF). Tumor size was estimated by caliper assessment of tumor radius and calculation of volume (see Methods). Results are presented in FIG. 8C.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating cancer in a human patient in need thereof, comprising the step of administering to the patient a therapeutically effective amount of devimistat and:
a. a fatty acid oxidation inhibitor, wherein the primary mechanism of action of the fatty acid oxidation inhibitor is not autophagy inhibition; or
b. a glycolysis inhibitor, wherein the primary mechanism of action of the glycolysis inhibitor is not autophagy inhibition.

2. (canceled)

3. A method for treating cancer in a human patient in need thereof, comprising the step of administering to the patient a therapeutically effective amount of devimistat, a fatty acid oxidation inhibitor, and a glycolysis inhibitor, wherein the primary mechanism of action of the fatty acid oxidation inhibitor, the glycolysis inhibitor, or both is not autophagy inhibition.

4. (canceled)

5. A method for treating cancer in a human patient in need thereof, comprising the step of administering to the patient a therapeutically effective amount of devimistat and
a. a tyrosine kinase inhibitor; or
b. a fatty acid oxidation inhibitor and a tyrosine kinase inhibitor.

6. The method of claim 1, wherein the fatty acid oxidation inhibitor is a peroxisomal fatty acid oxidation inhibitor.

7. The method of claim 6, wherein the peroxisomal fatty acid oxidation inhibitor is an ACOX1 inhibitor.

8. The method of claim 7, wherein the ACOX1 inhibitor is thioridazine.

9. The method of claim 1, wherein the glycolysis inhibitor is a glycogenolysis inhibitor.

10. The method of claim 9, wherein the glycogenolysis inhibitor is a glycogen phosphorylase inhibitor.

11. The method of claim 10, wherein the glycogen phosphorylase inhibitor is 5-chloro-N-[(1S,2R)-3-(dimethylamino)-2-hydroxy-3-oxo-1-(phenylmethyl)propyl]-1H-indole-2-carboxamide.

12. The method of claim 3, wherein the primary mechanism of action of the fatty acid oxidation inhibitor is lipophagy inhibition and the primary mechanism of action of the glycolysis inhibitor is not autophagy inhibition.

13. The method of claim 12, wherein the fatty acid oxidation inhibitor is hydroxychloroquine.

14. The method of claim 3, wherein the primary mechanism of action of the glycolysis inhibitor is autophagy inhibition and the primary mechanism of action of the fatty acid oxidation inhibitor is not autophagy inhibition.

15. The method of claim 14, wherein the glycolysis inhibitor is hydroxychloroquine.

16. The method of any of claim 5, wherein the tyrosine kinase inhibitor is a c-Met inhibitor.

17. The method of claim 16, wherein the tyrosine kinase inhibitor is crizotinib.

18. The method of claim 16, wherein the tyrosine kinase inhibitor is cabozantinib.

19. The method of claim 1, wherein the cancer is a cancer of the pancreas.

20. The method of claim 1, wherein the cancer is a cancer of the colon.

21. The method of claim 1, wherein the cancer is a cancer of the lung.

22. The method of claim 1, wherein the devimistat is administered orally.
